(19) United States
(12) Patent Application Publication
(10) Pub. No.: US 2020/0048686 A1
(43) Pub. Date: Feb. 13, 2020

(54) OLIGONUCLEOTIDE BINDING AGENTS

(71) Applicant: THE RNA MEDICINES COMPANY, INC., Bedford, MA (US)

(72) Inventors: Dalia Cohen, Bedford, MA (US); Michelle Markus, Bedford, MA (US); Jun Jiang, Bedford, MA (US); Michel Guiraldelli, Bedford, MA (US); Justin Boyd, Bedford, MA (US); Branko Radetich, Bedford, MA (US); Nanguneri Nirmala, Bedford, MA (US); Johan Pontin, Bedford, MA (US)

(73) Assignee: THE RNA MEDICINES COMPANY, INC., Bedford, MA (US)

(21) Appl. No.: 16/608,629

(22) PCT Filed: Apr. 26, 2018
(86) PCT No.: PCT/US18/29533

§ 371 Date: Oct. 25, 2019

Related U.S. Application Data
(60) Provisional application No. 62/490,362, filed on Apr. 26, 2017.

(51) Int. Cl. C12Q 1/6816; A61B 5/055

(52) U.S. Cl. CPC C12Q 1/6816; A61B 5/055

(57) ABSTRACT

The present disclosure relates to development and performance of screening methods capable of efficiently identifying candidate lead compounds that bind regulatory RNA oligonucleotides in a sequence-specific manner and exert a biological effect upon such regulatory molecules. Candidate lead compounds possessing RNA binding sequence specificity and targeted biological activity are described, as are approaches for merging structural, NMR-derived data with biological reporter assay results. Compound validation approaches capable of identifying the site(s) of action of such compounds within targeted RNA oligonucleotides are also provided.

### CROSS-REFERENCE TO RELATED APPLICATION

The present application is related to and claims priority under 35 U.S.C. § 119(e) to U.S. provisional patent application No. 62/490,362, entitled “Oligonucleotide Binding Agents,” filed Apr. 26, 2017. The entire content of the aforementioned patent application is incorporated herein by this reference.

### BACKGROUND OF THE INVENTION

RNAs, especially non-coding RNAs, have been identified as critical to regulation of gene expression, with particular relevance to various disease states (e.g., various microRNAs in particular have been identified as associated with a number of diseases, including cancer). While oligonucleotide-based agents (e.g., “antagomiRs”) that possess sequences complementary to regulatory RNAs (e.g., microRNAs) can be used to target/inhibit such regulatory RNAs in a sequence-specific manner, oligonucleotide therapeutics confront significant developmental and therapeutic hurdles related to delivery to a subject, as contrasted with small molecule inhibitors of regulatory RNAs. Small molecules that bind to and modulate regulatory RNAs can more efficiently be developed into therapeutic agents. There is therefore an unmet need for developing compound screening assays and identifying compounds by performance of such screening assays that can modulate the activity of regulatory RNAs with sequence specificity comparable to oligonucleotide therapeutics, while concurrently providing a significant delivery advantage over oligonucleotide therapeutics.

### SUMMARY OF THE INVENTION

The present disclosure is based, at least in part, upon the development and performance of screening methods capable of efficiently identifying candidate lead compounds that bind regulatory RNA oligonucleotides and exert a biological effect through such regulatory molecules. Certain aspects of the disclosure also provide the identity of such candidate lead compounds, their biological activity, and even identify the site of action for such candidate lead compounds within a targeted regulatory RNA oligonucleotide. Candidate lead compound identification and validation methods useful for merging structural, NMR-derived data with results obtained in biological reporter assays are also provided.

In one aspect, the disclosure provides a method for identifying a candidate lead compound involving: (a) contacting an oligonucleotide with a test compound in a biological functional assay, where oligonucleotide-test compound binding results in a biological result that is not observed in the biological functional assay without the test compound; and (b) contacting the oligonucleotide with the test compound, thereby forming an oligonucleotide-test compound solution; performing NMR upon the oligonucleotide-test compound solution, where oligonucleotide-test compound binding produces an NMR result not observed in a solution lacking the test compound; and detecting the NMR result in the oligonucleotide-test compound solution, where detecting selective biological function in the presence of the test compound in (a) and detecting the NMR result in (b) identifies the test compound as a candidate lead compound.

In one embodiment, the biological functional assay is a cell-based reporter system, a proliferation assay, an immunoassay or a polymerase chain reaction-based assay.

In another aspect, the disclosure provides a method for identifying a candidate lead compound involving (a) contacting an oligonucleotide with a test compound in the presence of a cell-based reporter system; and detecting selective activation of the cell-based reporter system in the presence of the test compound; and (b) contacting the oligonucleotide with the test compound, thereby forming an oligonucleotide-test compound solution; performing NMR upon the oligonucleotide-test compound solution, where oligonucleotide-test compound binding produces an NMR result not observed in a solution lacking the test compound; and detecting the NMR result in the oligonucleotide-test compound solution, where detecting selective activation of the cell-based reporter system in the presence of the test compound in (a) and detecting the NMR result in (b) identifies the test compound as a candidate lead compound.

In one embodiment, the oligonucleotide includes RNA. Optionally, the oligonucleotide is a coding or a non-coding RNA, optionally the oligonucleotide is a microRNA, a tRNA, a rRNA, a tiRNA, a lincRNA, a NAT, a lncRNA, a, eRNA, a T-UCR, a circRNA, a piRNA, an esiRNA, an siRNA, an antisense oligonucleotide, a tasiRNA, a snoRNA, a scaRNA or a snRNA.

In certain embodiments, the oligonucleotide is a microRNA. Optionally, the site of microRNA processing pathway activity of the candidate lead compound is assessed. In certain embodiments, assessing the site of microRNA processing pathway activity of the candidate lead compound involves measuring levels of pri-microRNA, pre-microRNA and mature microRNA, as compared to an appropriate control. In some embodiments, the candidate lead compound exhibits selective elevation or selective decrease of mature microRNA levels in cells administered the candidate lead compound, as compared to DMSO-treated control cells, optionally thereby indicating that the candidate lead compound interacts with the microRNA maturation pathway at the mature microRNA level. In related embodiments, the candidate lead compound exhibits selective elevation or selective decrease of pri-microRNA levels in cells administered the candidate lead compound, as compared to DMSO-treated control cells, optionally thereby indicating that the candidate lead compound interacts with the microRNA maturation pathway at the pri-microRNA level.

In another embodiment, the method further involves assessing the site at which the candidate lead compound binds the oligonucleotide, optionally involving assessing candidate lead compound binding following introduction of one or more mutant residues into the oligonucleotide. In certain embodiments, the candidate lead compound stabilizes a structural conformation of the oligonucleotide, optionally thereby inhibiting an activity of the oligonucleotide.

In one embodiment, the NMR result not observed in a solution lacking the test compound is the presence of an NMR peak observed for the oligonucleotide-test compound solution that is not observed in a control solution lacking the test compound.

In another embodiment, the oligonucleotide is a microRNA-21 transcript or a fragment thereof including at least 15 consecutive nucleotides of microRNA-21. In a related embodiment, the microRNA-21 transcript sequence fragment includes the Drosha cleavage site of a microRNA-21 transcript. In certain embodiments, the microRNA-21 transcript sequence fragment includes the Dicer cleavage site of a microRNA-21 transcript.

In one embodiment, the cell-based reporter system is a fluorescent protein reporter system (optionally a GFP, CFP, BFP, RFP and/or YFP reporter system) or is a luciferase reporter system. Optionally, the cell-based reporter system is a luciferase reporter system involving a vector encoding for both firefly luciferase and renilla luciferase, optionally where firefly luciferase is operably linked to a microRNA-complementary sequence, optionally where the microRNA-complementary sequence is positioned at the 3′-terminus of the firefly luciferase open reading frame, optionally where the microRNA-complementary sequence is fused to the 3′-terminus of the firefly luciferase transcript, optionally where the microRNA-complementary sequence is a microRNA-21 complementary sequence.

In certain embodiments, the test compound is a small molecule.

In another embodiment, detecting selective activation of the cell-based reporter system in the presence of the test compound in step (a) includes assigning a numeric score to the test compound-oligonucleotide reporter system results.

In one embodiment, detecting selective activation of the cell-based reporter system in the presence of the test compound in step (a) involves identifying at least a 1.5-fold elevation of the level of a signal in the presence of the test compound, relative to the level of the signal in the absence of the test compound, optionally detecting selective activation of the cell-based reporter system in the presence of the test compound in step (a) involves identifying at least a 1.75-fold elevation of the level of a signal in the presence of the test compound, relative to the level of the signal in the absence of the test compound, optionally detecting selective activation of the cell-based reporter system in the presence of the test compound in step (a) involves identifying at least a two-fold elevation of the level of a signal in the presence of the test compound, relative to the level of the signal in the absence of the test compound.

In some embodiments, the test compound inhibits microRNA-21 activity by at least 1.5-fold more than the level of microRNA-21 activity in control cells in the absence of the test compound, optionally the test compound inhibits microRNA-21 activity by at least 1.75-fold more than the level of microRNA-21 activity in control cells in the absence of the test compound, optionally the test compound inhibits microRNA-21 activity by at least two-fold more than the level of microRNA-21 activity in control cells in the absence of the test compound, optionally where the control cells are DMSO-treated. In a related embodiment, the numeric score assigned to the test compound-oligonucleotide reporter system results is on a 10 point scale.

In one embodiment, the test compound is assigned (i) an integer score based upon the shape of a dose-response curve for the biological functional assay in the presence of the test compound and (ii) an integer score based upon the dose-responsiveness of the biological functional assay to the test compound. Optionally, the test compound is assigned (i) an integer score between 0 and 3 based upon the shape of a dose-response curve for the cell-based reporter system in the presence of the test compound (optionally, where 0 is given to test compounds that show no signal; 1 indicates a signal only at higher concentrations; 2 indicates a signal proportional to its concentration; and 3 indicates a signal at low concentrations) and (ii) an integer score between 0 and 7 based upon the dose-responsiveness of the cell-based reporter system to the test compound (optionally, where the higher value is assigned when a signal is shown at lower concentrations).

In certain embodiments, oligonucleotide-test compound binding is identified by detecting shifting of peaks in NMR spectra in the presence of a test compound, as compared to a control NMR spectra in the absence of the test compound.

In another embodiment, the test compound is assigned an integer score between 0 and 3 based upon the shape of a dose-response curve for the cell-based reporter system in the presence of the test compound, as represented below:

and the test compound is assigned an integer score between 0 and 7 based upon the following dose-response criteria in the cell-based reporter system in the presence of the test compound:

| Activity | Score |
|---|---|
| ≥2-fold activity change at 0.1 μM or less | 7 |
| ≥2-fold activity change at 0.3 μM to 0.1 μM | 6 |
| ≥2-fold activity change at 1 μM to 0.3 μM | 5 |
| ≥2-fold activity change at 3 μM to 1 μM | 4 |
| ≥2-fold activity change at 10 μM to 3 μM | 3 |
| ≥2-fold activity change at 30 μM to 10 μM | 2 |
| ≥2-fold activity change at 100 μM to 30 μM | 1 |
| ≥2-fold activity change at >100 μM or not observed | 0. |

In some embodiments, detecting the NMR peak in the oligonucleotide-test compound solution in (b) involves assigning a numeric score to the oligonucleotide-test compound NMR results. Optionally, a score of 0, 1, 2, 3 or 4 is assigned to the oligonucleotide-test compound NMR results, where a score of 4 indicates high-quality NMR-detected binding, optionally where scoring is assigned as follows:
+1 for signal demonstrating binding;
+1 for signal-to-noise ratio >3;
+1 for sharp ligand peaks in the mixture;
+1 for pattern of ligand peaks consistent with the pattern expected from the chemical structure.

In certain embodiments, the method further involves assigning a numeric score of 0, 1, 2, 3 or 4 to the oligonucleotide-test compound NMR results, where a score of 4 indicates high-quality NMR-detected binding. In a related embodiment, a combined score of the biological functional assay and the NMR assay of 10 or greater identifies the test compound as a compound that binds the oligonucleotide and/or is bioactive.

In some embodiments, a method of the disclosure involves (a) contacting an oligonucleotide with a test compound in the presence of a cell-based reporter system; and detecting selective activation of the cell-based reporter system in the presence of the test compound; and (b) contacting the oligonucleotide with the test compound, thereby forming an oligonucleotide-test compound solution; performing NMR upon the oligonucleotide-test compound solution, where oligonucleotide-test compound binding produces an NMR result not observed in a solution lacking the test compound; and detecting the NMR result in the oligonucleotide-test compound solution, where detecting selective activation of the cell-based reporter system in the presence of the test compound in (a) and detecting the NMR result in (b) identifies the test compound as a candidate lead compound.

Certain embodiments further involve performing a cellular proliferation assay upon the test compound.

In some microRNA embodiments, the method further involves identifying whether the test compound binds to a double stranded fragment including one or more of the Drosha cleavage site of a microRNA transcript, a fragment including the Dicer cleavage site of a microRNA transcript, or both, optionally where NMR is performed upon a Dicer cleavage site-containing structure.

In certain embodiments, the method further involves validating binding of the test compound to the oligonucleotide by performance of NMR upon oligonucleotide-test compound solutions titrated across multiple test compound concentrations. Optionally, the NMR results performed upon oligonucleotide-test compound solutions titrated across multiple test compound concentrations show dose-responsiveness.

In one embodiment, the test compound is selected from a library of test compounds. Optionally, the test compound library is assembled using chemoinformatics and/or crystallography.

Another aspect of the disclosure provides a test compound library of less than 1000 compounds, where the library includes at least 10 or more test compounds selected by a chemoinformatics and/or crystallography process to be likely oligonucleotide-binding compounds.

An additional aspect of the disclosure provides a method for validating a candidate lead compound and identifying the site of candidate lead compound-microRNA interaction involving: identifying binding of a candidate lead compound to a microRNA or a microRNA fragment; altering the sequence of the microRNA or microRNA fragment via introduction of one or more point mutations, thereby generating a mutated microRNA or a mutated microRNA fragment; and identifying absence of binding of the candidate lead compound to the mutated microRNA or mutated microRNA fragment, thereby validating the candidate lead compound and identifying the site of candidate lead compound-microRNA interaction.

A further aspect of the invention provides a method for validating a candidate lead compound and identifying the site of activity of the candidate lead compound within the microRNA pathway involving: identifying binding of a candidate lead compound to a microRNA or a microRNA fragment; assaying levels of pri-microRNA, pre-microRNA and mature microRNA in the presence of the candidate lead compound, as compared to in the absence of the candidate lead compound; and identifying the site of activity of the candidate lead compound within the microRNA pathway based upon the relative levels of pri-microRNA, pre-microRNA and mature microRNA assayed in the presence of the candidate lead compound, as compared to in the absence of the candidate lead compound, thereby validating the candidate lead compound and identifying the site of activity of the candidate lead compound within the microRNA pathway.

### Definitions

By “agent” is meant any small compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The disclosure also provides the use of derivatives of the disclosed compositions, such as salts with physiologic organic and inorganic acids like HCl, H₂SO₄, H₃PO₄, malic acid, fumaric acid, citronic acid, tartaric acid, and acetic acid.

“Detect” refers to identifying the presence, absence, or amount of the polypeptide, nucleic acid (e.g., DNA, RNA, microRNA, rRNA, etc.) and/or other composition/substance/moiety to be detected.

By “effective amount” is meant the amount of an agent required to ameliorate the symptoms of a disease relative to an untreated patient. The effective amount of active agent(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a nucleic acid or polypeptide molecule. This portion contains, preferably, at least 10%, 20%, 30%, 40%, 50%, 60%, 70%, 80%, or 90% of the entire length of the reference nucleic acid molecule or polypeptide. A fragment may contain 5, 7, 10, 20, 30, 40, 50, 60, 70, 80, 90, 100, 200, 300, 400, 500, 600, 700, 800, 900, or 1000 or more nucleotides or amino acids.

By “gene” is meant a locus (or region) of DNA that encodes a functional RNA or protein product, and is the molecular unit of heredity.

As used herein, the term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than (“concentrated”) or less than (“separated” or “diluted”) than that of its naturally occurring counterpart.

By “marker” is meant any protein or polynucleotide having an alteration in expression level or activity that is associated with a disease or disorder.

By “modulate” is meant alter (increase or decrease). Such alterations are detected by standard art known methods such as those described herein.

“Non-naturally occurring” as applied to an oligonucleotide means that the oligonucleotide contains at least one moiety that is different from the corresponding wildtype or native oligonucleotide sequence. Non-natural sequences (e.g., sequences comprising nucleotides that do not occur in corresponding native sequence(s)) can be determined by performing BLAST search using, e.g., the lowest smallest sum probability where the comparison window is the length of the sequence of interest (the queried) and when compared to the non-redundant (“nr”) database of Genbank using BLAST 2.0. The BLAST 2.0 algorithm is described in Altschul et al. (1990) J. Mol. Biol. 215:403-410. Software for performing BLAST analyses is publicly available through the National Center for Biotechnology Information.

By “nucleic acid” is meant biopolymers, or large biomolecules, essential for all known forms of life. Nucleic acids, which include DNA (deoxyribonucleic acid) and RNA (ribonucleic acid), are made from monomers known as nucleotides. Each nucleotide has three components: a 5-carbon sugar, a phosphate group, and a nitrogenous base. If the sugar is deoxyribose, the polymer is DNA. If the sugar is ribose, the polymer is RNA. Together with proteins, nucleic acids are the most important biological macromolecules; each are found in abundance in all living things, where they function in encoding, transmitting and expressing genetic information—in other words, information is conveyed through the nucleic acid sequence, or the order of nucleotides within a DNA or RNA molecule. Strings of nucleotides strung together in a specific sequence are the mechanism for storing and transmitting hereditary, or genetic information via protein synthesis. Nucleic acids include but are not limited to: deoxyribonucleic acid (DNA), ribonucleic acid (RNA), double-stranded DNA (dsDNA), single-stranded DNA (ssDNA), messenger RNA (mRNA), ribosomal RNA (rRNA), transfer RNA (tRNA), micro RNA (microRNA), and small interfering RNA (siRNA).

By “nucleic acid sequence” is meant a succession of letters that indicate the order of nucleotides within a DNA (using GACT) or RNA (GACU) molecule. By convention, sequences are usually presented from the 5′ end to the 3′ end. For DNA, the sense strand is used. Because nucleic acids are normally linear (unbranched) polymers, specifying the sequence is equivalent to defining the covalent structure of the entire molecule. For this reason, the nucleic acid sequence is also termed the primary structure. The sequence has capacity to represent information. Biological DNA represents the information which directs the functions of a living thing. In that context, the term genetic sequence is often used. Sequences can be read from the biological raw material through DNA sequencing methods. Nucleic acids also have a secondary structure and tertiary structure. Primary structure is sometimes mistakenly referred to as primary sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

As used herein, “obtaining” as in “obtaining an agent” includes synthesizing, purchasing, or otherwise acquiring the agent.

By “small molecule” is meant a compound typically characterized in that it contains several carbon-carbon bonds, and has a molecular weight of less than 5000 Daltons (5 kD), optionally less than 3 kD, optionally less than 2 kD, and in certain embodiments, less than I kD. In some cases, a small molecule has a molecular weight equal to or less than 700 Daltons, optionally equal to or less than 500 Daltons.

By “subject” is meant a mammal, including, but not limited to, a human or non-human mammal, such as a bovine, equine, canine, ovine, murine, rattus or feline.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

A “therapeutically effective amount” is an amount sufficient to effect beneficial or desired results, including clinical results. An effective amount can be administered in one or more administrations.

Any compositions or methods provided herein can be combined with one or more of any of the other compositions and methods provided herein.

Other features and advantages of the disclosure will be apparent to those skilled in the art from the following detailed description and claims.

### BRIEF DESCRIPTION OF THE DRAWINGS

FIG. 1 depicts a schematic overview of the approach to the present disclosure. FIG. 1 shows the relationship of applying functional biology, structural biology and chemistry to screening, validating and optimizing microRNA (microRNA) molecules as an exemplary target oligonucleotide. The approach of the present disclosure combines NMR and biological functional assays in a platform to identify compounds that bind oligonucleotides, including, e.g., RNAs, microRNAs and/or non-coding RNAs in a rapid manner. In some embodiments, the present disclosure describes targeting of oncomirs (oncogenes) that drive tumor development and/or may be involved in other diseases.

FIG. 2 depicts a schematic overview of the microRNA pathway and also shows (at right) Antagomir-21 inhibitory/degradative activity upon mature microRNA-21. As described herein, microRNA is a small non-coding RNA molecule (containing about 19-22 nucleotides in its mature form) which functions in RNA silencing and post-transcriptional regulation of gene expression. The human genome may encode over 2500 microRNAs, which are abundant in many mammalian cell types and appear to target at least 60% of the genes of humans and other mammals.

FIGS. 3A and 3B depict expression of microRNA-21 in cancer and that anti-microRNA-21 agents exhibit anti-tumor therapeutic benefit. FIG. 3A is a graph demonstrating microRNA-21 profiles for all stages of breast cancer, as compared to levels in normal solid tissue. The dark line in each box represents the median. FIG. 3B is a series of photographs demonstrating expression of microRNA-21 in microRNA-21 induced B-cell lymphoma. Tumor bearing mice were examined with and without microRNA-21 overexpression (at day 0, microRNA-21 was overexpressed, but reduction of microRNA-21 was then achieved via use of Cre and Tet-off technologies (Medina et al. Nature 467: 86-90), and the time course was monitored). A loss of microRNA-21 was witnessed over time to result in shrinkage of B-cell lymphoma tumors at days 2-6, as shown. As demonstrated in FIGS. 3A and 3B, microRNA-21 has been found to be overexpressed in different cancers. The anti-tumor benefit observed for reduction of microRNA-21 levels demonstrates that microRNA-21 represents not just a biomarker for disease but also a target for treatment.

FIG. 4 depicts an illustration showing that microRNA-21 has been identified as overexpressed in many cancers, including glioblastoma, breast cancer, lung cancer, prostate cancer, stomach cancer, colon cancer, cervical cancer, and head and neck cancer.

FIG. 5 depicts a schematic of the role in microRNA-21 in tumor suppression. microRNA-21 plays a role in inhibition of RECK, Maspin, MARKs, TPM1, PTEN, and PDCD4 (Programmed Cell Death 4, a neoplastic transformation inhibitor).

FIGS. 6A and 6B depict diagrams demonstrating constructs for parallel compound screening. FIG. 6A depicts a diagram of a reporter gene cell based assay used in an MCF-7 breast cancer cell line, for identification of agents capable of suppressing microRNA-21 activity. The construct includes a microRNA-21 complementary sequence as well as reporter sequences for firefly luciferase and renilla luciferase (with renilla luciferase acting as a transfection control). Expression of antagomiR-21 acts as a positive control, turning on firefly luciferase expression in the reporter assay via release of microRNA-21 inhibition of firefly luciferase expression. The expression cassette may include sequences complementary to other microRNAs, as an alternative or even in addition to microRNA-21. FIG. 6B depicts microRNA stem loop structures and the location of sequences within the RNA structure that are targeted by Dicer and Drosha. These structures were employed for NMR detection assays, in compound screening methods described herein. The red sequences represent the mature/functional microRNA-21.

FIGS. 7A and 7B depict histograms, a 96-well plate layout and diagrams of primary compound screening. FIG. 7A presents a histogram and diagram of a 96 well plate, with the histogram demonstrating that inhibition of microRNA-21 by an antagomiR-21 construct increased reporter gene detection by approximately 50-fold. A scrambled antagomiR-21 sequence did not lead to expression of the reporter gene (microRNA-21 inhibition of firefly luciferase expression was maintained at baseline levels). FIG. 7B is a diagram of the screening scheme used to detect compounds that bind RNA molecules. As described herein, cells that represent a disease model (e.g., MCF-7 breast cancer cells) were plated on Day 1. On Day 2, reporter constructs containing a complementary sequence to miR-21 or a scrambled sequence in the 3′ UTR of Firefly microRNAs (e.g., microRNA-21; pmicroRNA-GLO) were transiently transfected into the disease model cell line. Transfected cells were treated with compounds to be screened at different concentrations (e.g., 10 μM, 1 μM, etc.). On Day 5, the reporter assay (e.g., Dual-GLO assay) was performed and data was analyzed. The reporter enzymes (renilla and firefly luciferase) were evaluated at 48 hours after compound treatment (the Day 5 mark).

FIG. 8 depicts a series of diagrams that define the criteria for primary compound screening selection. As described herein, hits are identified as any compound that inhibits microRNA activity (e.g., microRNA-21 activity) in cell culture by at least 1.5-fold or greater, as compared to cells treated with DMSO alone. The fold change is determined by comparing total average normalized luciferase activity of compound treated cells to normalized luciferase activity of DMSO treated cells. As described herein, 93 compounds (13%) of a 696 compound library exhibited readings above this threshold.

FIGS. 9A and 9B depict the NMR approach employed for primary screening of compounds targeting RNA molecules. FIG. 9A is a diagram depicting an exemplary NMR spectrum obtained during the small molecule library screen and a Venn diagram showing classification of the corresponding hits identified by the NMR screen. Using NMR, peaks that corresponded to compounds binding Dicer or Drosha constructs indicated a positive hit. As described herein, subsets of tested compounds were classified according whether they bind Dicer, Drosha, or to both constructs. FIG. 9B depicts a diagram showing the overlap of hits that were identified according to their performance in cell-based assays and in NMR profiling. Subsets of compounds regarded as positive hits under both NMR and cell-based assays were selected for immediate further examination.

FIGS. 10A, 10B and 10C depict a series of chromatograms and graphs that demonstrate the scoring methods as described herein. FIG. 10A is a sample NMR spectrum depicting peaks and how they would be scored. FIG. 10B depicts a series of curves for the reporter gene assay. As described herein, compounds are assigned a score based on graphs of curves of the intensity of light units at different compound concentrations, as compared to a control curve. Light units are measured based on raw firefly luminescence data. FIG. 10C depicts a graph demonstrating the potency score used in the reporter assay screen. Luciferase is normalized to Renilla and the fold change is determined by comparing normalized compound samples relative to DMSO control samples. As described herein, compounds are assigned a potency score based on the lowest concentration that achieves a 2-fold or higher change in activity. More potent compounds receive a higher score, while less potent compounds have a lower score. As demonstrated in FIGS. 10B and 10C, there is a bias towards selecting compounds with higher activity at lower concentrations. For scoring, a maximum 14 point scale scoring system is based on 4 possible points from NMR screens and 10 possible points from the reporter gene assay based on the dose response observed (including 0-3 points for curve shape, as in FIG. 10B, and 0-7 points assessed for concentration level at which two-fold increase in light units is first achieved).

FIG. 11 depicts a series of screened compounds identified as hits (potential leads), along with their molecular weight, NMR score, functional score, total score, IC50 and IC25. Compounds with total scores from 9 to 13 are depicted.

FIGS. 12A and 12B depict a series of schematics that demonstrate the microRNA pathway and possible points of agent-mediated microRNA-21 modulation. FIG. 12A depicts a schematic of the entry points for small molecules modulating microRNA (e.g., microRNA-21) maturation and/or activity of mature microRNAs (schematic adapted from the Sharp lab website at web.mit.edu/sharplab/researchsummary.html). FIG. 12B depicts the microRNA pathway and points at which a test compound may act on microRNAs. Graphs depict the results of the reporter assay of test microRNA antagonists (i.e., antagomiR, e.g., antagomiR-21), as compared to untreated and scrambled control for pri-microRNA, pre-microRNA, and mature-microRNA (e.g., pri-microRNA-21, pre-microRNA-21, and mature-microRNA-21). As demonstrated in FIG. 12B, the antagomiR-21 inhibits the activity of microRNA-21 at the mature-microRNA-21 stage, likely by promoting degradation of mature microRNA-21.

FIGS. 13A and 13B depict graphs demonstrating the results of reporter and proliferation assays performed on a positive anti-microRNA-21 compound. FIG. 13A depicts the reporter assay results including graphs of raw luciferase activity and the fold change relative to DMSO for the compound (BSI101023) at different concentrations used for scoring. FIG. 13B depicts the proliferation assay results measuring the percent cell count confluency of compound BSI101023 at different concentrations compared to DMSO. As demonstrated herein, cell growth declines in a dose dependent manner with increasing concentration of the test compound BSI101023.

FIGS. 14A to 14C depict additional biological assays for testing the functionality of a microRNA-binding compound. FIG. 14A depicts a scratch assay used to test the compound BSI101023. The compound was analyzed for the ability to block re-population of cells at different compound concentrations, as compared to a DMSO-treated control. FIG. 14B depicts the downstream effects of microRNA-21 antagonism with test compounds BSI101023, BSI101534, and BSI101484. Introduction of BSI101023 has similar effects on microRNA-21 as those observed for a positive control antagomiR-21 oligonucleotide agent. FIG. 14B demonstrates that compounds BSI101023 and BSI101484 increased the expression of Programmed Cell Death 4 (PDCD4), a downstream effector of microRNA-21, consistent with the anti-microRNA-21 activity observed. FIG. 14C shows a proliferation and apoptosis analysis of MCF-7 cells treated with an antagomir-21 oligonucleotide.

FIGS. 15A and 15B depict quantitative analysis of microRNA processing species at IC₂₅. FIG. 15A depicts a schematic of the microRNA-21 pathway and the point at which the antagomiR-21 compound BSI101023 was identified to act. FIG. 15B is a graph depicting the percent expression of pri-microRNA-21, pre-microRNA-21, and mature-microRNA-21 relative to DMSO control after cell exposure to BSI01023, or to antagomiR. *P<0.05. ***P<0.0005. NS is “not significant”. While antagomiR-21 was observed to cause a significant reduction in mature microRNA-21 (consistent with sequestering and degradation of mature microRNA-21 by the antagomiR), mature microRNA-21 was observed to accumulate when BSI101023 was administered (suggesting that RISC-loading of mature microRNA-21 was blocked by BSI101023).

FIGS. 16A, 16B and 16C depict graphs demonstrating the results of reporter, proliferation, and downstream effector assays performed on a positive anti-microRNA-21 compound. FIG. 16A depicts the reporter assay results including graphs of raw luciferase activity and the fold change relative to DMSO for the compound (BSI101484) at different concentrations, which were used for scoring of test compounds. FIG. 16B depicts the proliferation assay results measuring the percent confluency of compound BSI101484 at different concentrations, as compared to DMSO-treated cells. As demonstrated herein, cell growth declined in a dose dependent manner with increasing concentration of potential compound behaving as an anti-microRNA BSI101023. FIG. 16C depicts the downstream effects of microRNA-21 antagonism when test compounds BSI101023, BSI101484, and BSI101534 were administered. Introduction of BSI101484 had similar effects upon microRNA-21, as compared to BSI101023 and a positive control (antagomiR-21). FIG. 16C demonstrates that compounds BSI101023 and BSI101484 also increased the expression of Programmed Cell Death 4 (PDCD4), a downstream effector of microRNA-21.

FIGS. 17A and 17B depict schematics and results of apoptosis assays in MCF-7 cells. FIG. 17A shows that treatment with BSI101484 or 101023 caused dose dependent apoptosis in MCF-7 in 120 hrs. FIG. 17B shows a real time apoptosis assay, performed using IncuCyte Zoom and caspase-3/7 reagent.

FIGS. 18A and 18B depict a schematic of the microRNA processing pathway and quantitative analysis of microRNA processing species for various anti-microRNA-21 agents, relative to DMSO-treated control cells, at IC25. FIG. 18A depicts a schematic of the microRNA-21 pathway and the point at which the anti-microRNA-21 compound BSI101484 likely acts. FIG. 18B is a graph depicting the percent expression of pri-microRNA-21, pre-microRNA-21, and mature-microRNA-21 relative to DMSO-treated control cells, after exposure to antagomiR-21 as a positive control, anti-microRNA-21 compound BSI101484, or to no antagomiR. ***P<0.0005. NS is “not significant”.

FIGS. 19A to 19E depict a series of schematics and spectra that demonstrate the confirmation of NMR titration from screening to initial SAR. FIG. 19A depicts the scheme for performing NMR analysis of positive hits in the top panel. In the bottom panel, the structure of double stranded RNA targeted by anti-microRNA agents is shown. In some embodiments, the mechanism of how a test compound inhibits microRNA involves an agent acting as and/or mimicking a wobble base and through binding to microRNA, creating a stable form of microRNA that is not cleaved by Dicer or Drosha. FIG. 19B depicts a microRNA-21 stem loop structure and NMR spectra obtained for a microRNA-21-interacting test compound, BSI101534. As demonstrated in the NMR spectra, there was a shift of in peaks corresponding to bases U24 and G27 observed in the presence of BSI101534, the effect of which was dose-dependent, consistent with BSI101534 binding microRNA-21. BSI101534 binds only to the Dicer target sequence-containing construct, in the A26 bulge region, as demonstrated by mapping the positions of U24 and G27 onto the stem-loop diagram. FIG. 19C depicts the microRNA stem loop diagrams and NMR spectra obtained for test compounds BSI101484 and BSI101023. Compounds BSI101484 and BSI101023 also bind to the A26 bulge region. FIG. 19D depicts the microRNA stem loop diagram and NMR spectra for a fragment of test compound BSI101484—called BSI104171. FIG. 19E depicts a NMR spectra and stem loop diagram using naturally occurring nucleobases (D58) as a control. No peaks shift when the nucleobases are added. Thus, no binding was observed between the U, T, A, and G nucleobases and/or nucleosides tested with a naturally-occurring nucleobase (D58).

FIGS. 20A and 20B depict stem loop diagrams and NMR spectra obtained in examining the role of the effects of the sequence context of the A26 bulge on binding of certain anti-microRNA-21 test compounds. FIG. 20A shows NMR spectra for test compounds BSI101534, BSI101484, and BSI 104171 mixed with an RNA construct lacking the A-bulge (D57). The RNA peaks do not shift in the presence of any of the three compounds; there is no binding. Therefore, it was concluded that the A26 bulge region was necessary for binding. FIG. 20B depicts additional structures and NMR spectra for the test compound BSI101534. By changing the length of the microRNA but retaining the A26 bulge region, it was demonstrated that the A26 region is sufficient for microRNA-21 binding by the anti-microRNA-21 test compounds.

FIGS. 21A to 21H depict stem loop diagrams and NMR spectra that demonstrate whether the binding of test compounds to Dicer-containing microRNAs is sequence-context dependent. FIG. 21A depicts a scheme for mutational mapping of local sequence context, to identify context relevant to microRNA-binding test compounds. In altered microRNA-21-derived constructs, sequences in the bulge region were substituted to mimic bulge regions from non-microRNA-21s (in FIG. 21A, hsa-mir-103a-1 and a mutant form thereof, as shown). Additionally, sequences outside the bulge region were altered to mimic the non-bulge regions of other non-microRNA-21s. FIG. 21B depicts alternate stem loop schemes and NMR spectra for microRNA-21-derived constructs where 1) the neighboring base pairs remain but the bulge is altered (“hsa-mir-16-1”) and 2) neighboring base pairs are altered while the bulge region is altered, yet the bulge nucleotide is retained (“hsa-mir-155”). NMR spectra the different altered microRNA-21-derived constructs are depicted as BSI101534 and BSI104171 are added. Peaks that shift when both BSI101534 and BSI104171 are added to the hsa-mir-16-1 construct (retaining neighboring base pairs) are shaded. The compounds bind to the construct with the U bulge in the same sequence context as miR21 but not to the A bulge in the miR155 sequence context. FIG. 21C depicts further changes to the sequence context based on the has-mir-155 construct from FIG. 21B. When the upper base pair flanking the A-bulge is restored to GC (“hsa-mir-155-mut”), binding by BSI101534 and BSI104171 is restored somewhat. FIG. 21D depicts stem loop diagrams and NMR spectra for microRNA-21-derived constructs where both the bulge region and sequences outside the bulge region are altered to those of hsa-mir-103a-1. NMR spectra of the RNA in the presence of BSI101534 and BSI104171 are shown. Unexpectedly, the compounds bind to hsa-mir-103a-1, even though the bulge and flanking base pairs are different from mir-21. FIG. 21E shows that hsa-mir-103a-1 can adopt an alternative base-pairing scheme compared to that predicted by mfold. The alternative scheme preserves the CG base pairs flanking the bulge, similar to the mir21 context. This account for the binding in the mir-103 context. FIG. 21F depicts stem loop diagrams and NMR spectra for the microRNA-21-derived hsa-mir-103a-1 construct of FIG. 21D, with a mutation to lock the hairpin into the structure predicted by mfold. With the hairpin locked into the predicted miR103 secondary structure, BSI101534 and BSI104171 no longer bind (no shift observed). FIG. 21G depicts stem loop structures and NMR spectra for a microRNA-21-derived structure having the hsa-mir-200c bulge region sequence, which possesses a mismatch instead of the A bulge. NMR spectra for this construct in the presence of BSI101534 and are shown. No binding was observed for either compound. FIG. 21H presents a summary of exploration of the sequence context for compound binding.

FIG. 22 shows the NOESY (Nuclear Overhauser effect spectroscopy) spectrum for the miR21 D29 hairpin. NOE crosspeaks are observed for sequential bases G27 and A28, and G66 and A67, demonstrating that these bases are close together in space (within 5 Å), as expected from the sequence and the predicted helical structure. Strikingly, crosspeaks are observed for the bulge base, A26, to both G27 and G66, suggesting that the base stacks into the helix. A model, based for the structure, based on pdb entry 5A17, is shown.

FIGS. 23A and 23B depict schematics for the likely binding mode for compounds to the microRNA-21 bulge region. The flanking GC/CG base pairs stabilize the helix. The bulge nucleotide flips in and interacts with the compound. The compound makes favorable stacking interaction with the neighboring base pairs. If the compound is extended, there is the potential to make additional favorable interactions with the rest of the RNA. FIG. 23B shows crosspeaks identified in 2D NOESY spectrum.

FIG. 24 depicts a schematic showing the hit finding and lead generation process employed.

FIGS. 25A and 25B show two hits identified by the parallel screening process. FIG. 25A shows the diiminoisoindoline hit, BSI101484. A search of the in-house library identified the analog, BSI104171. A summary of the results for both compounds in the reporter assay and NMR is presented in the table. FIG. 25B shows the 4-Hydroxyquinoline hit, BSI101534. Again, searching the in-house library turned up analogs BSI101536 and BSI101473, which were also tested. The results are presented in the table.

FIG. 26 depicts stem loop diagrams of microRNA-21 constructs prepared for crystallization. Based upon a 103-mer microRNA-21 full length construct, three microRNA-21 constructs were set up for crystallization. These include a microRNA-21 58-mer “Dicer construct”, a microRNA-21 45-mer Dicer tetraloop, and a microRNA-21 two strand stem of 21 and 22-mers (a duplex comprising a 21mer and a 22mer, with bulge nucleotide as shown).

FIGS. 27A and 27B depict graphs demonstrating differentially expressed microRNAs in Stage 2b breast cancer patients. FIG. 27A depicts the median microRNA counts per million microRNA reads found in stage 2b primary breast tumor samples. FIG. 27B depicts the fold changes observed in stage 2b primary tumor compared to normal tissue for various microRNAs. Differential expression of the microRNAs shown are statistically significant with adjusted p-values<0.001. All fold changes shown are greater than 2.5 or less than -2.5.

FIG. 28 depicts a graph demonstrating microRNA-21 expression levels observed in breast cancer, by stage (TCGA). Elevated microRNA-21 expression was identified across all stages examined.

FIG. 29 depicts the downstream effects of microRNA-21 antagonism with test compounds BSI101023, BSI101534, BSI100945, and BSI101484. Administration of BSI101023 exhibited similar effects on microRNA-21 levels as a positive control, antagomiR-21. Compounds BSI101023 and BSI101484 specifically increased the expression of Programmed Cell Death 4 (PDCD4), a downstream effector of microRNA-21.

FIG. 30 presents a list of suitable miRNA targets expressly contemplated for application of the methods and compositions of the instant disclosure, together with disease targets expressly contemplated for each such suitable miRNA target.

### DETAILED DESCRIPTION OF THE INVENTION

The present disclosure is based, at least in part, upon the development and successful performance of screening methods that efficiently identify candidate lead compounds that bind regulatory RNA oligonucleotides (e.g., microRNAs) and exert a biological effect upon such regulatory molecules. Certain aspects of the disclosure provide specific candidate lead compounds that were discovered to bind and inhibit microRNA-21 in a sequence-specific manner, thereby identifying compounds likely to exert therapeutic effects upon microRNA-21 overexpressing cancers (e.g., glioblastoma, breast cancer, lung cancer, prostate cancer, stomach cancer, colon cancer, cervical cancer, and head and neck cancer). Certain aspects of the disclosure also provide validation methods capable of identifying the specific site(s) of action for such candidate lead compounds within a targeted regulatory RNA oligonucleotide. Test compound screening methods capable of merging structural, NMR-derived data with results obtained in biological reporter assays in an efficient and effective manner to identify candidate lead compounds that bind and modulate activity of regulatory RNA oligonucleotides (e.g., microRNAs and other non-coding RNAs) are also provided. The parameters of the disclosure are set forth in additional detail below.

### Methods of the Present Invention

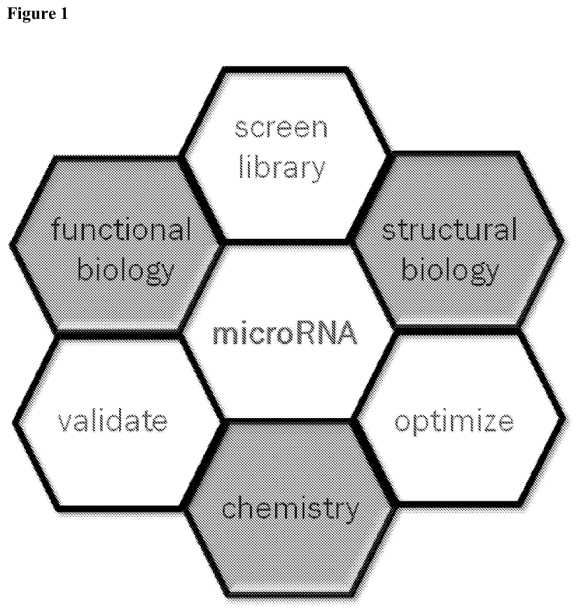

The present disclosure describes a parallel integrative approach to identifying novel compounds—particularly small molecules—which bind and/or inhibit oligonucleotides, e.g., microRNA function. Fields of genetics/bioinformatics, functional biology, structural biology (NMR and/or crystallography), medicinal chemistry, and cheminformatics may be combined to identify and validate compounds which bind oligonucleotides, e.g., compounds that bind and disrupt microRNAs relevant to different diseases states. FIG. 1 shows the relationship of applying functional biology, structural biology and chemistry to screening, validating and optimizing compounds that bind microRNA (microRNA) molecules. The approach of the present disclosure combines NMR and biological functional assays in a harmonized platform to identify rapidly highly effective compounds that bind RNAs, microRNAs and non-coding RNAs. In certain embodiments, the present disclosure describes methods of inhibiting oncomirs (oncogenes) that drive tumor development and/or may be involved in other diseases.

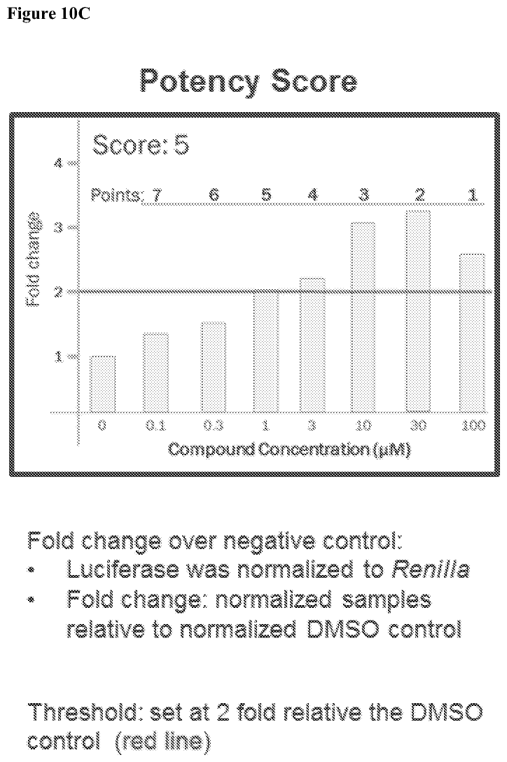

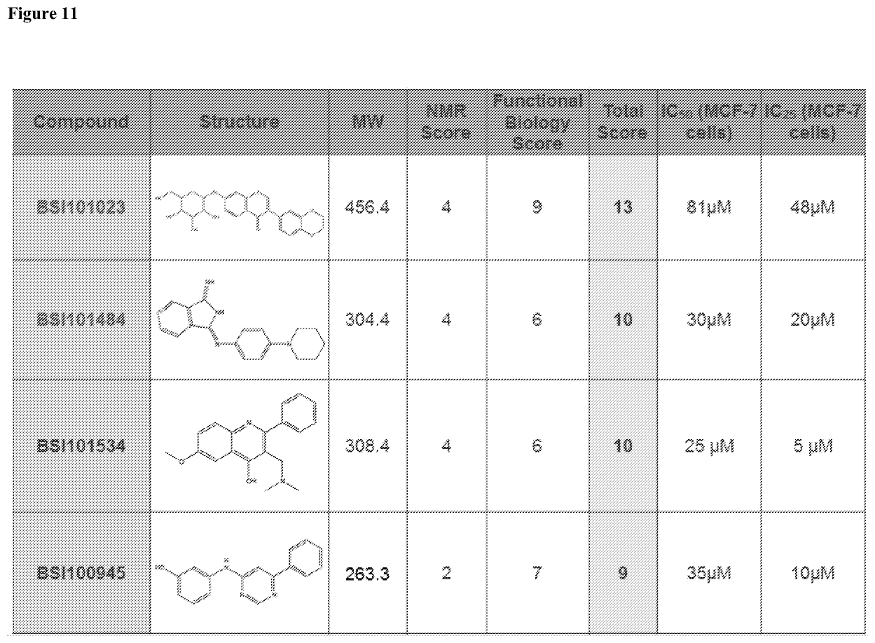

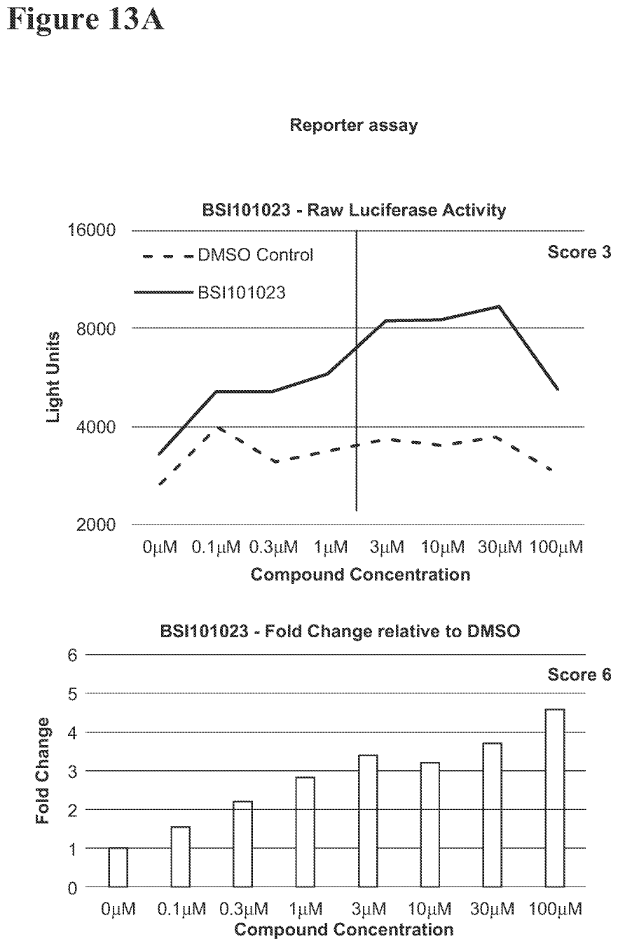

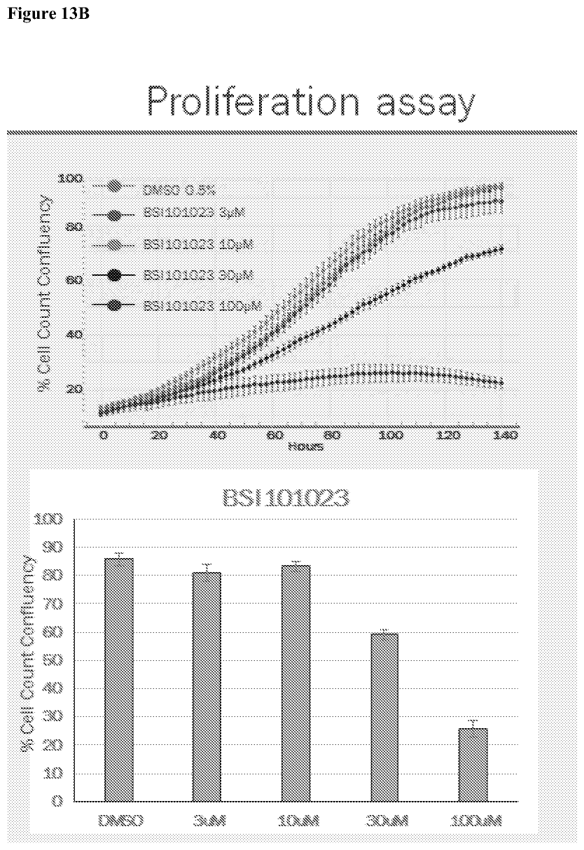

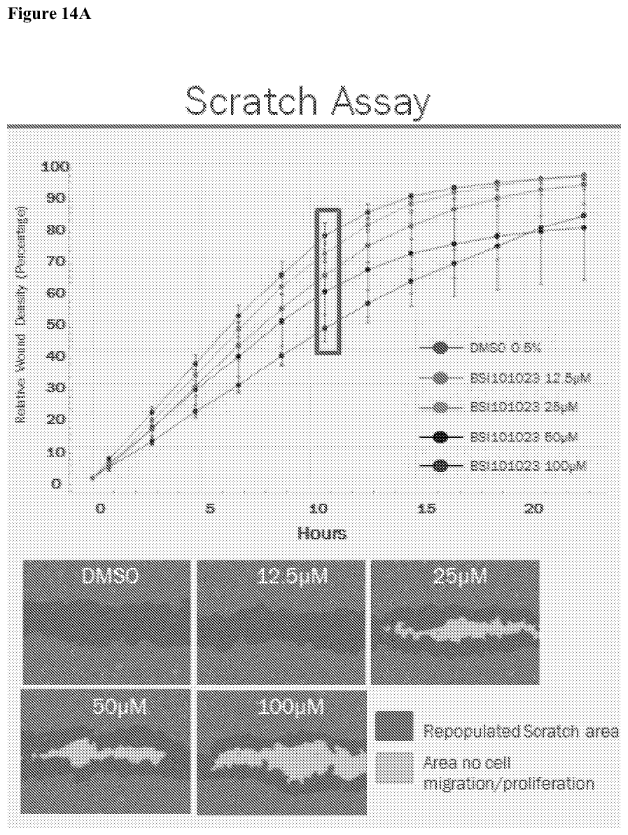

Conveniently, the compounds of the present invention may be numerically scored using the present methods to identify compounds that bind and modulate the activity of, for example inhibit, oligonucleotides such as RNA, specifically microRNA. For each assay used herein, a numeric value is assigned based on the likelihood that the result indicates a positive binding and/or inhibition of the compound to the oligonucleotide and preferably at lower concentrations of the compound. The values may then be combined to generate a score for each compound such that a high score indicates a high likelihood of favorable binding characteristics such as specificity, for example as detected by NMR, and ability to inhibit the activity of the oligonucleotide at useful concentrations, for example using a biological functional assay. FIG. 10 in particular provides examples of such scoring techniques, and FIGS. 11 and 13 provides additional examples of scoring.

In one embodiment, NMR is used to identify compounds that bind an oligonucleotide, such as microRNA, based on peaks in the spectrum generated when the compound is in the presence of the oligonucleotide. A peak appearing at greater than 1% of the reference spectrum indicates that binding between compound and oligonucleotide is detected. Such a peak is assigned a score of +1. A signal to noise ratio of greater than 3.0 indicates signal above noise is significant. This is assigned an additional score of +1. Sharp peaks indicate nonspecific binding is excluded; this is also assigned a score of +1. The spectrum is compared to that predicted from the compound's chemical structure and concentration. Agreement indicates that the good compound integrity and solubility. This is assigned a score of +1. Based on these parameters, a screened compound has a possible NMR score of 0-4, with a score of 4 indicating high quality binding.

In another embodiment or in addition to the NMR assay described above, a biological functional assay is used to identify compounds that modulate the function of the oligonucleotide, such as inhibit the activity of microRNA. Compounds can inhibit microRNAs at various stages of the microRNA pathway including: at the level of transcription and/or the initial transcript (pri-microRNA), pre-microRNA, microRNA duplex, and mature RNA. Inhibition may take place inside the nucleus and/or in the cytoplasm. Inhibition can be detected by cell based assays, for example using cell lines transfected with a reporter construct that includes an oligonucleotide sequence complementary to the microRNA of interest, for example miR-21, which is linked to a reporter gene such as luciferase or a fluorescent protein such as GFP and similar. In this type of assay, the reporter gene expression is silenced in the presence of the active microRNA of interest, or target microRNA, and activated when the microRNA of interest is inhibited by binding of a suitable compound. Inhibition is measured by comparing the signal generated by cells treated with the test compound against that generated in suitable positive (known microRNA antagonists) and negative controls. Inhibition can be described as a statistically significant increase of the signal compared to the negative control, for example the signal is at least 1.25-fold, at least 1.5-fold, at least 1.75-fold, at least 2-fold, at least 2.5-fold, at least 3-fold, etc, greater than the negative control. FIG. 10C provides an example of such a comparison. The greater the inhibition, the more potent the compound is, and compounds can be assigned a potency score. In one example, a potency score of 0 to 7 is assigned to the compound, with 7 being the most potent. Alternative assays may be used, for example proliferation assays, scratch assays, non-cell based assays such as immunoassays such as Western blots, ELISAs, and bead based assays such as A1phaLISA™ (Perkin Elmer), as well as PCR assays such as RT-PCR, qPCR and the like.

Furthermore, compounds can be assigned a score based on the shape of curves of the signal generated in the biological functional assay at different compound concentrations as compared to the negative control. This curve-based score biases the overall score towards compounds that have a higher activity at lower concentration. As exemplified in FIG. 10B, compounds are given low scores for showing no inhibition or inhibition only at higher concentrations. A higher score is given to compounds that show increased activity at higher concentrations, while the highest score is given to compounds that show strong inhibition at low concentrations, for example below a set threshold of desirable concentration. In this example, a dose response score is given between 0 (no inhibition) and 3 (high inhibition at low concentration).

Thus, by combining the scores from the NMR and biological functional assays, test compounds can be evaluated for their ability to bind oligonucleotides, for example microRNA, as well as the quality of that binding in a simple, consistent and easy to compare manner. Test compounds that have a good composite score may be then be designated candidate lead compounds.

It can be appreciated that using the methods of the present invention, not only gross binding of the compound to oligonucleotides, for example microRNA, can be detected, but also the specific binding site can be identified by providing one or more specific target sequences corresponding to different regions of the oligonucleotide. For example, binding pri-microRNA, pre-microRNA, microRNA duplex, and mature microRNA can be detected, as well as binding to certain key sequences within the oligonucleotide, such as the Dicer and/or Drosha cleavage sites of microRNA. Inhibition of the microRNA can also be measured by measuring transcription, translation and degradation to determine at which step the inhibition occurs. Such methods can be used to validate and characterize test compounds and candidate lead compounds.

Regulatory RNA Oligonucleotides (e.g., microRNAs) as Therapeutic Targets

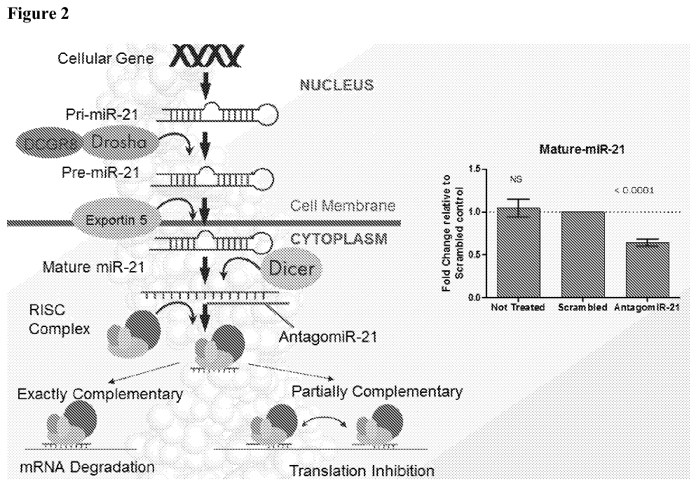

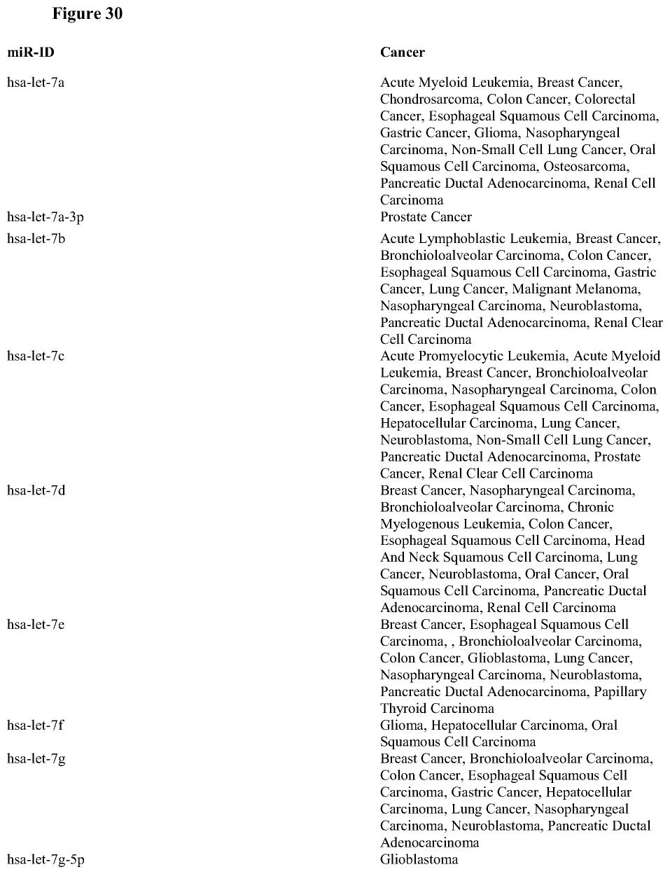

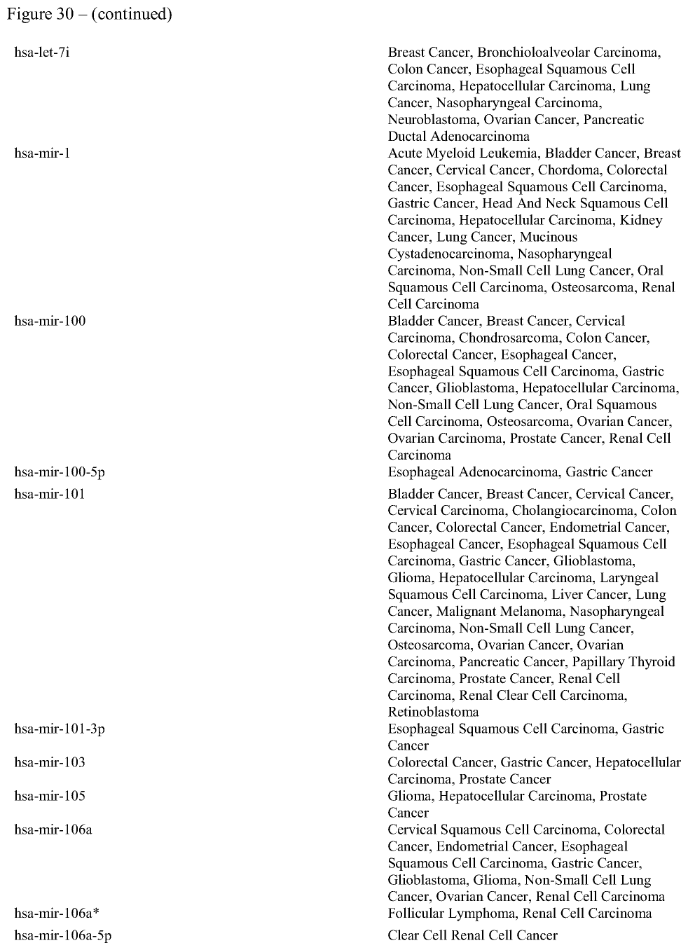

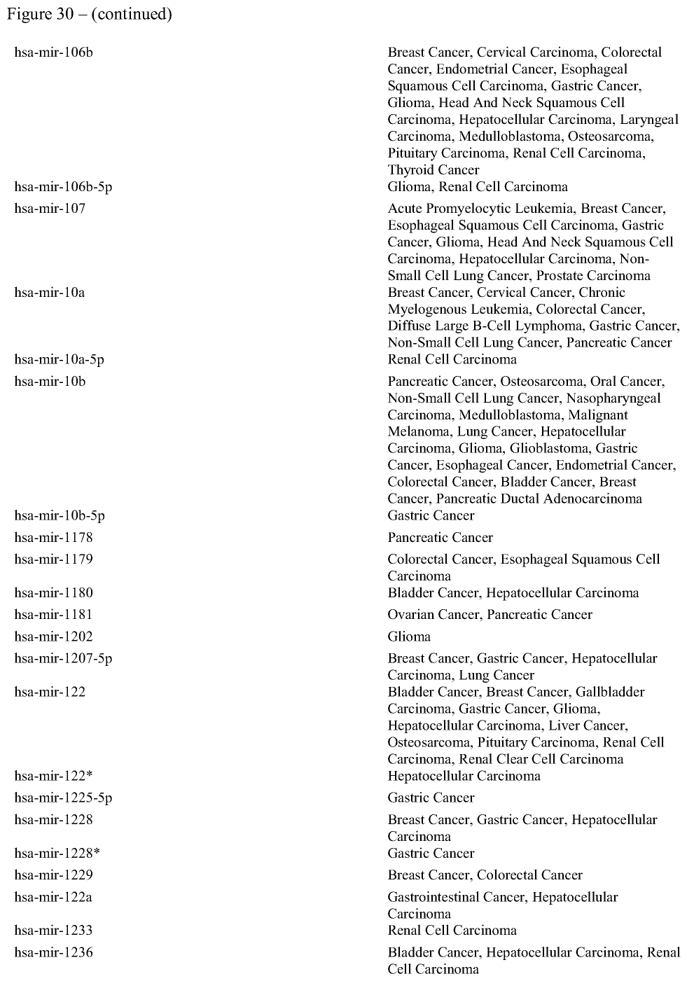

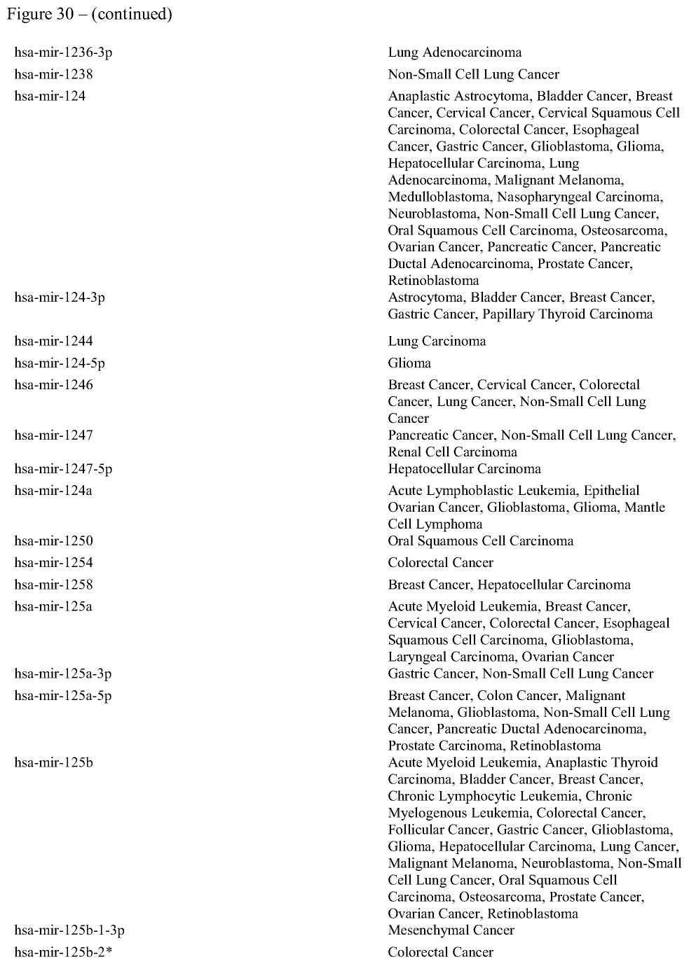

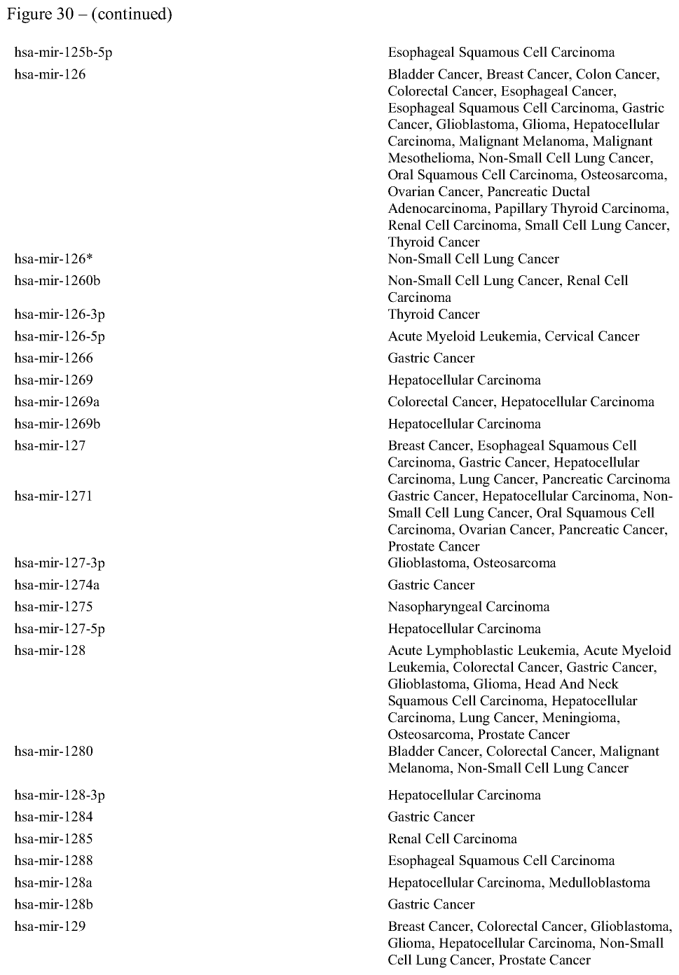

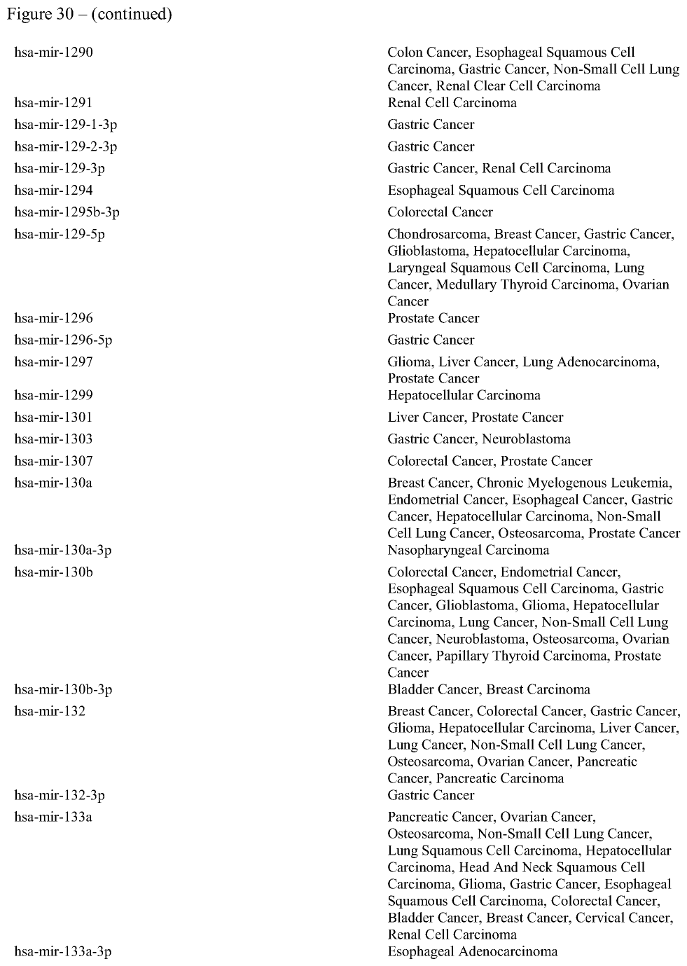

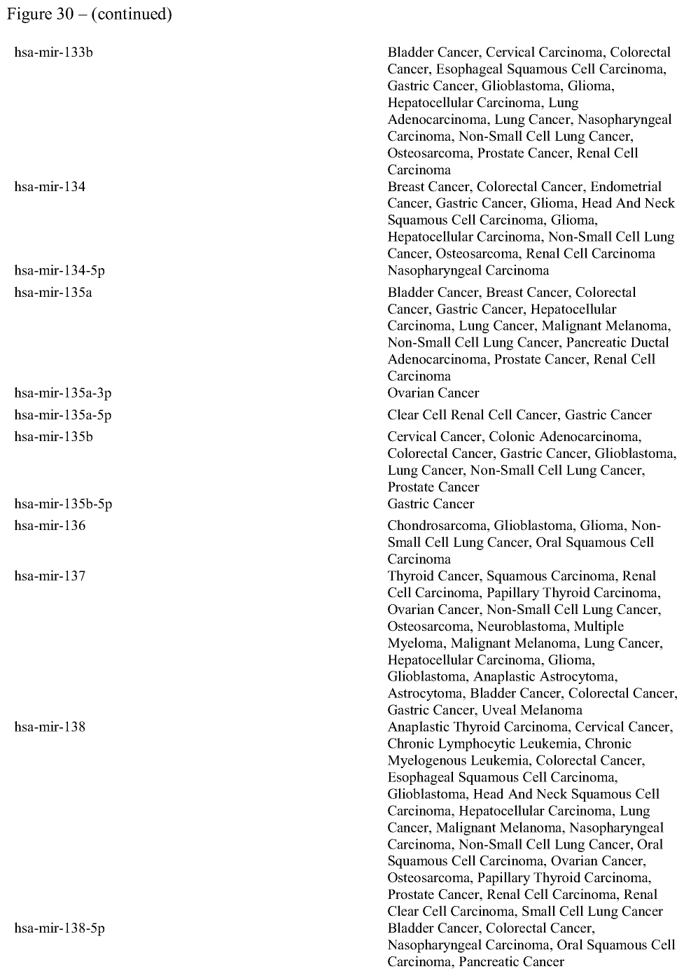

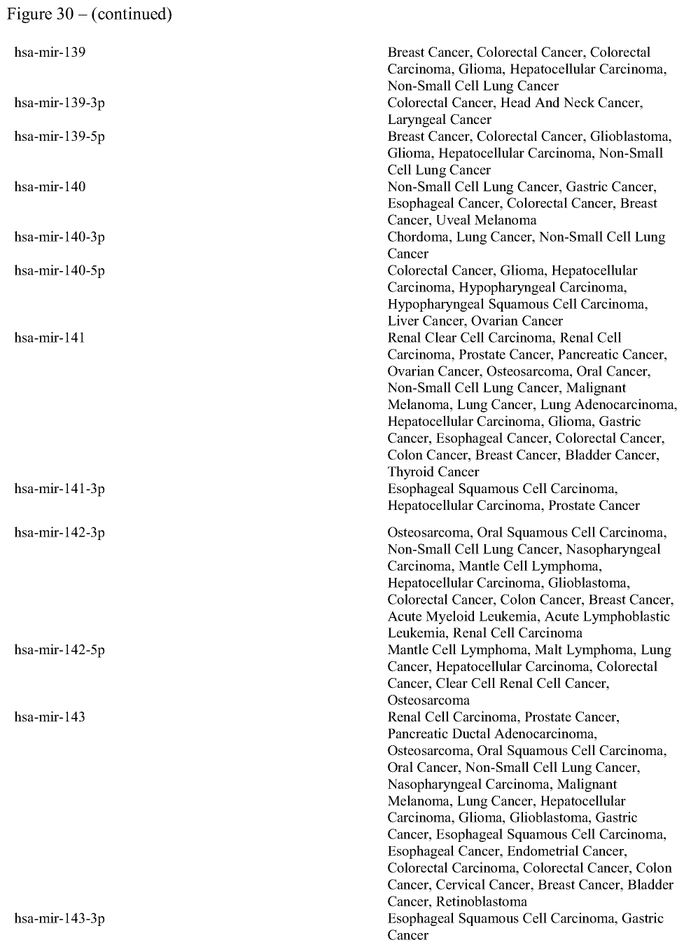

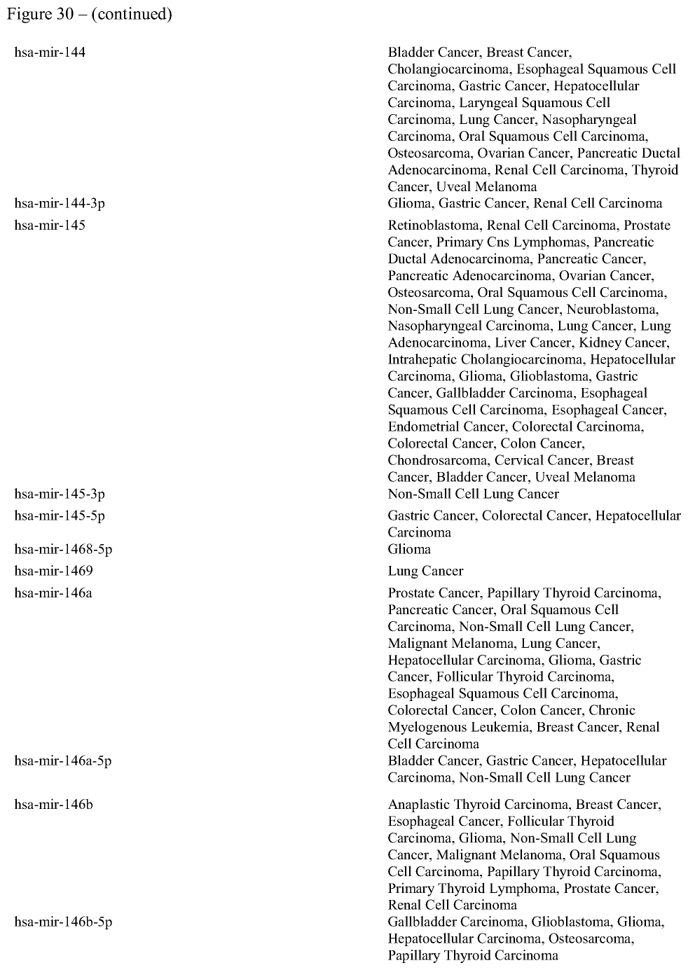

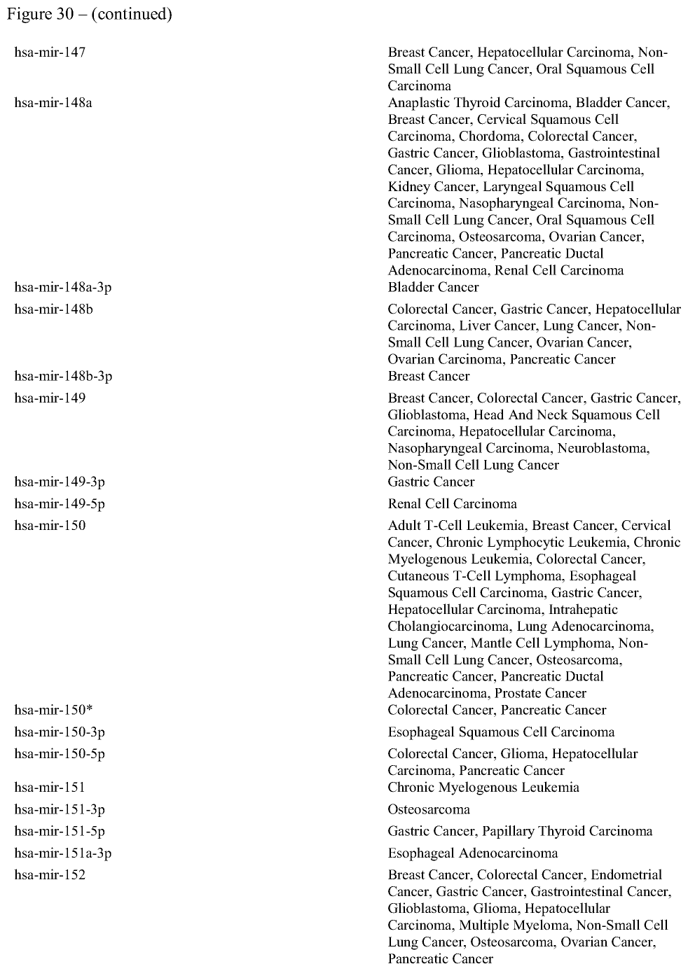

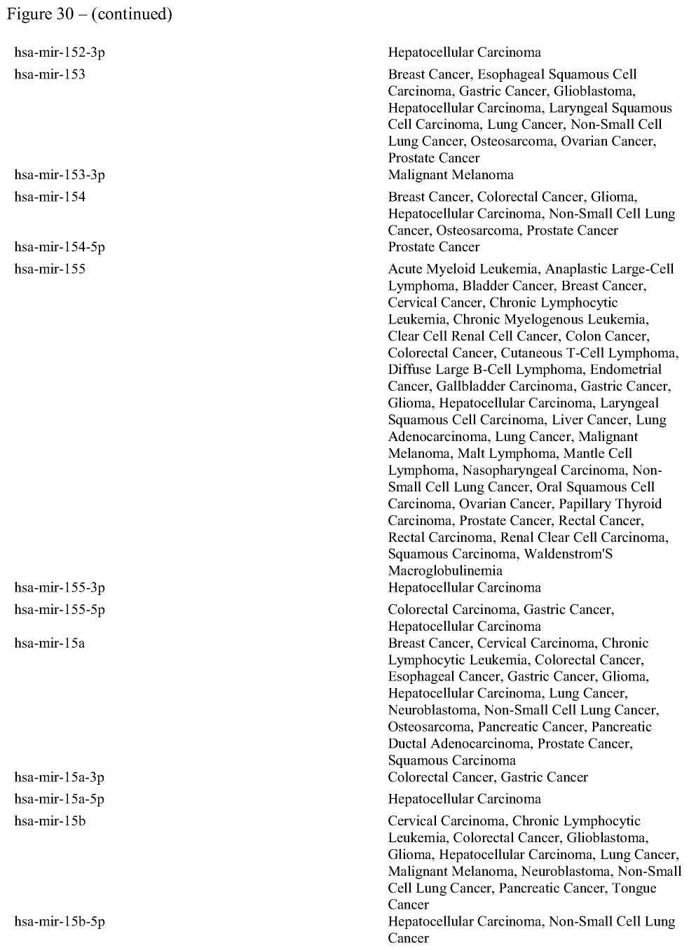

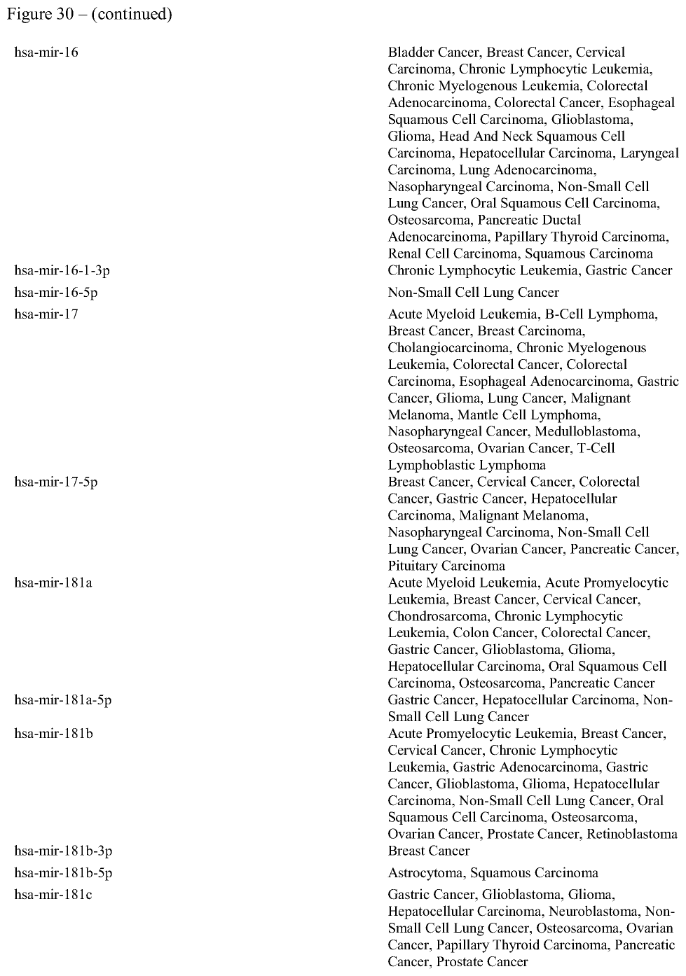

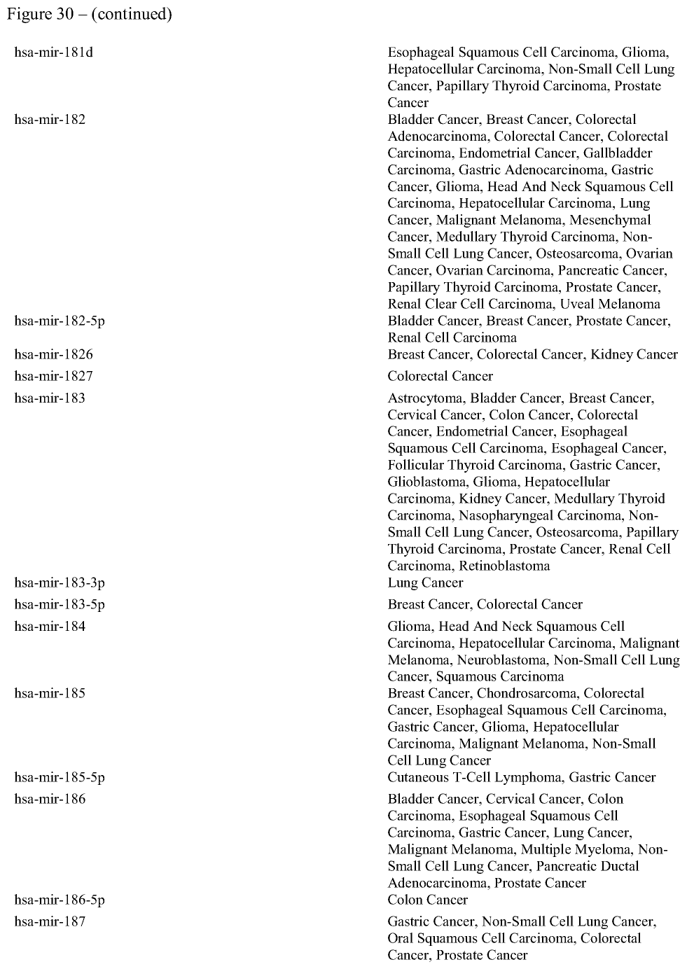

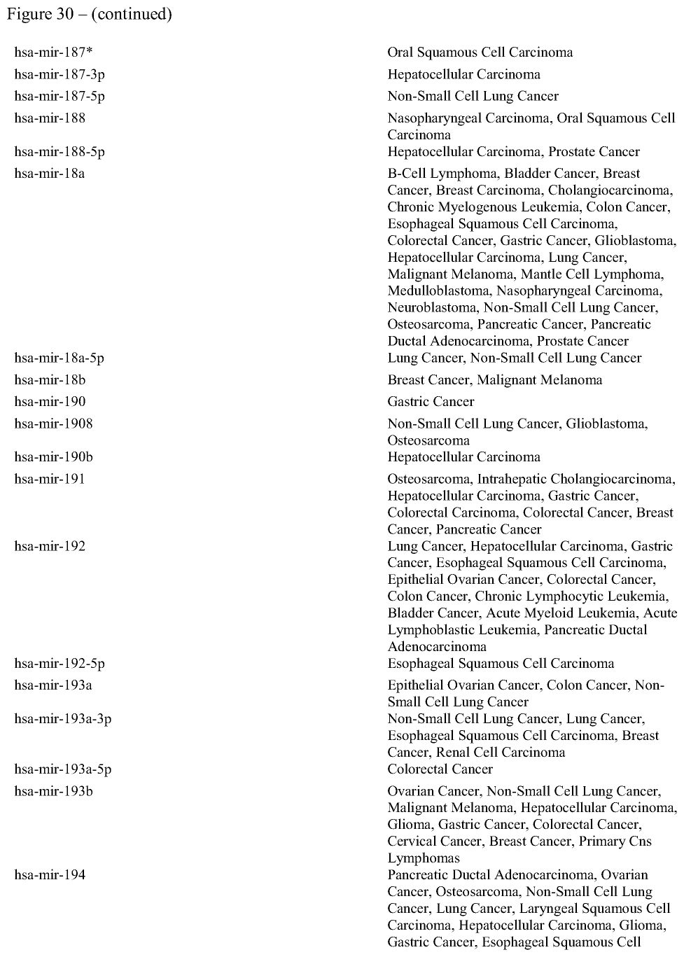

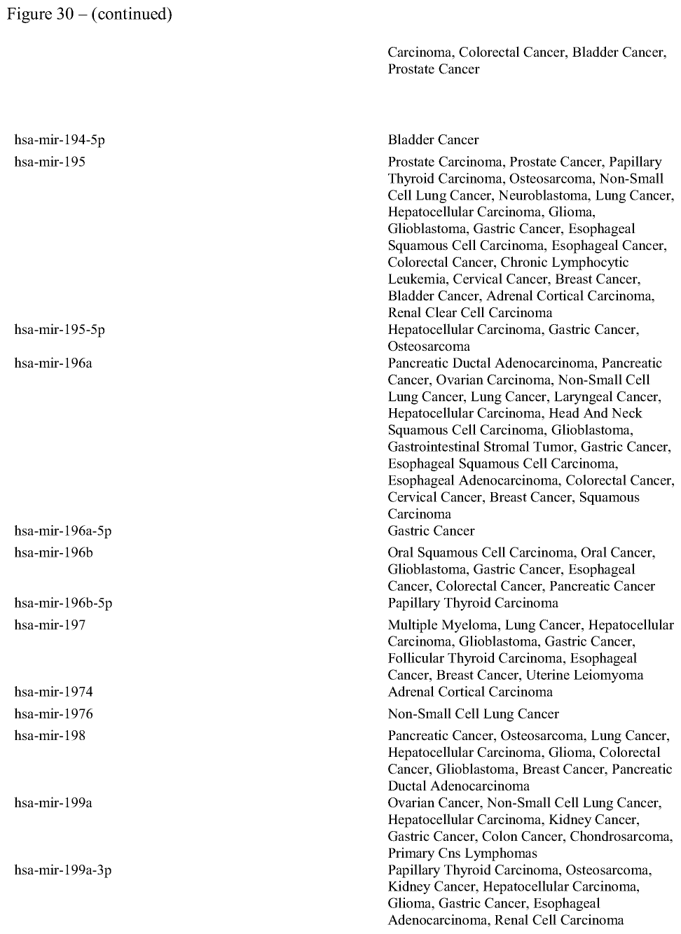

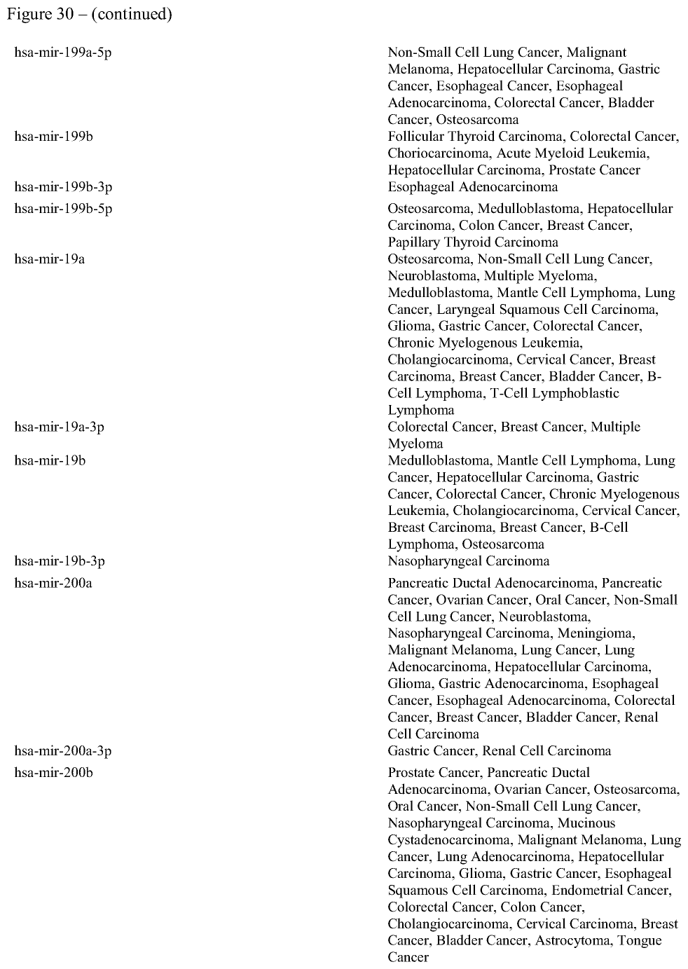

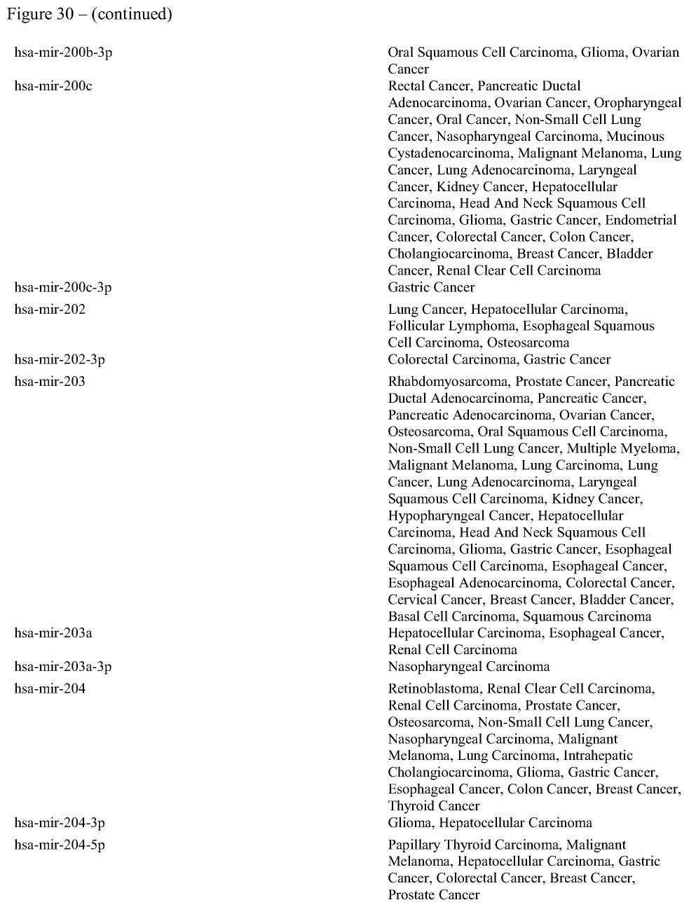

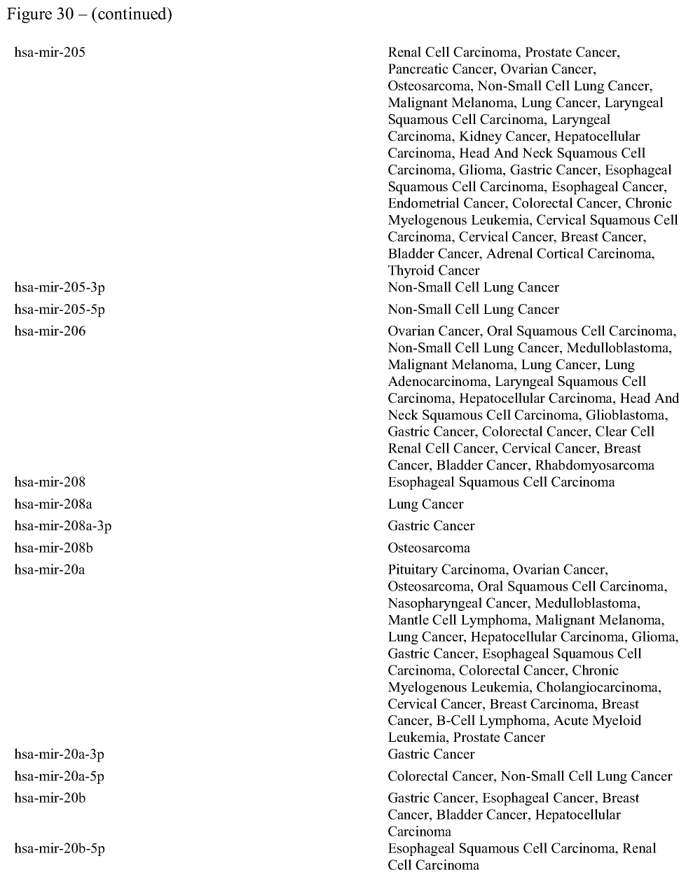

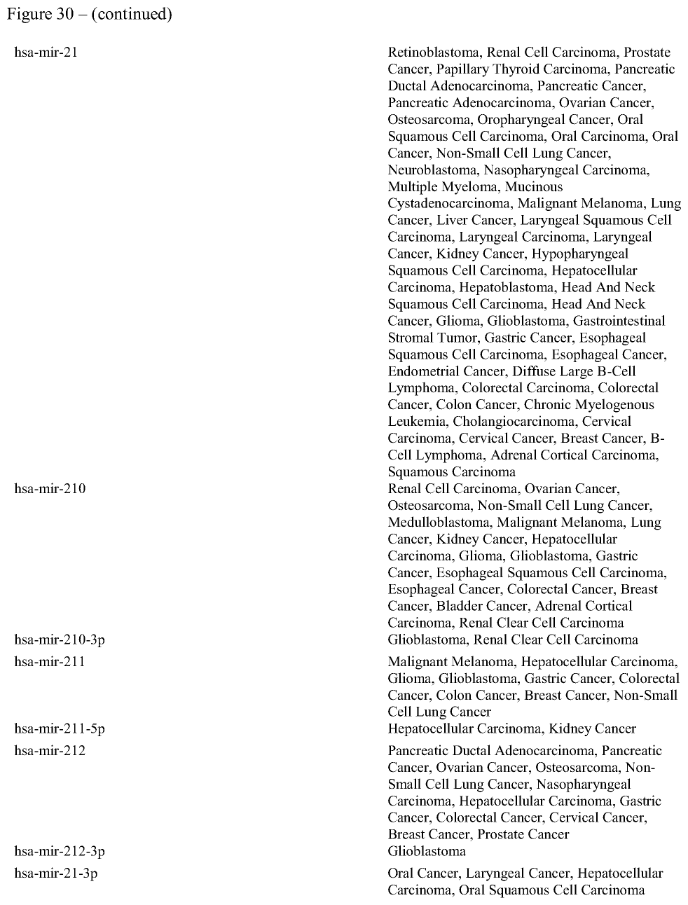

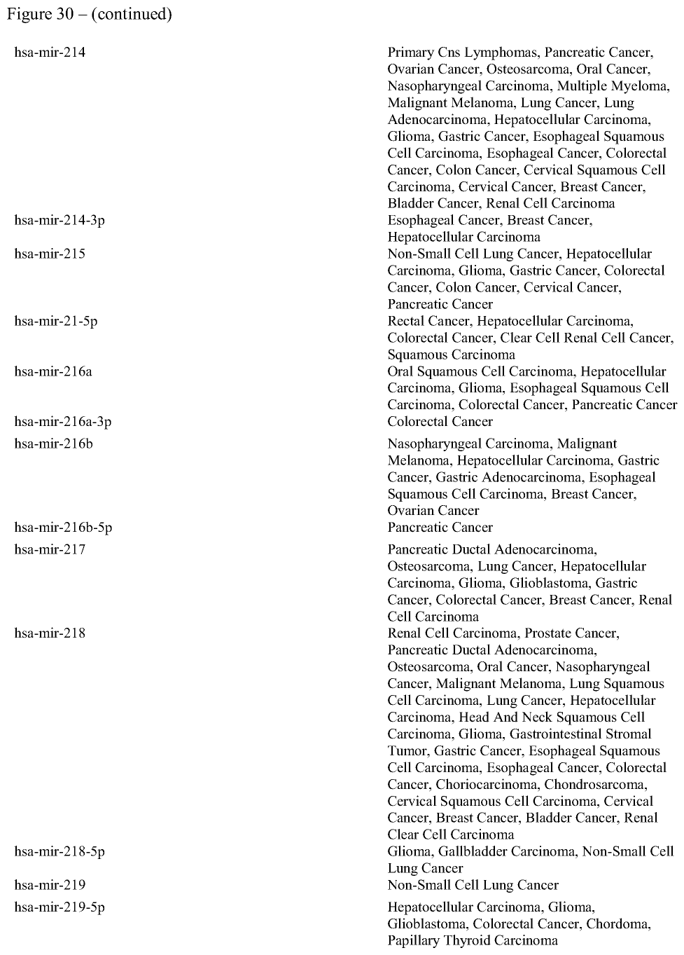

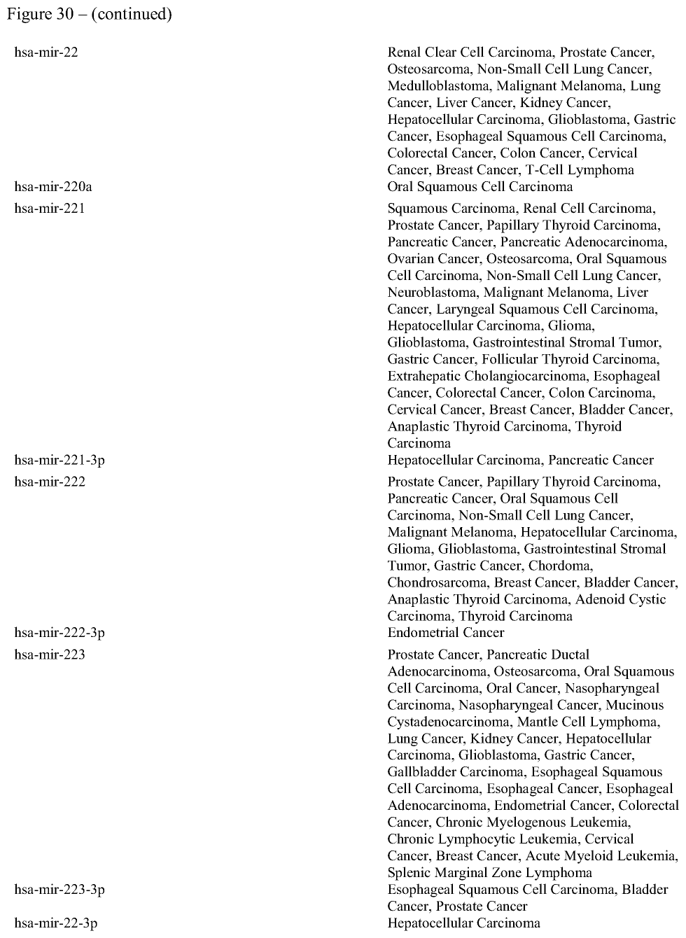

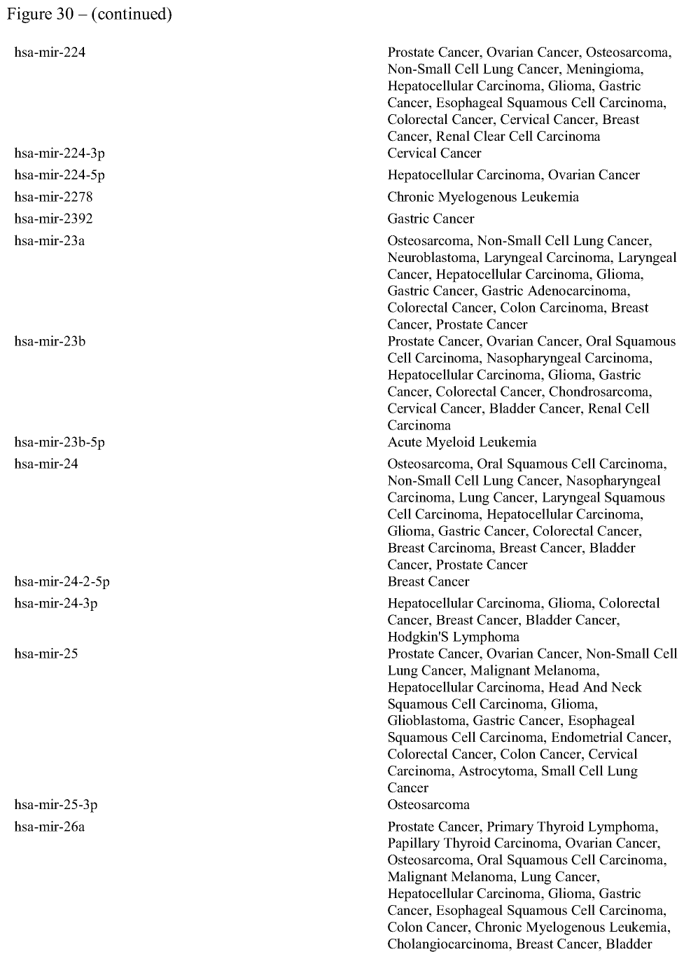

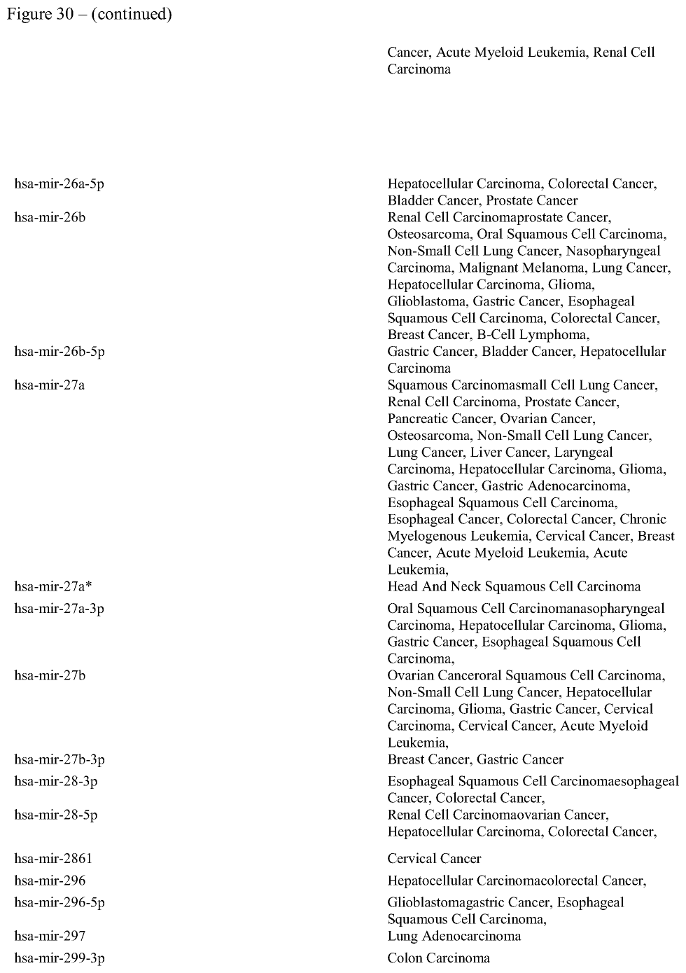

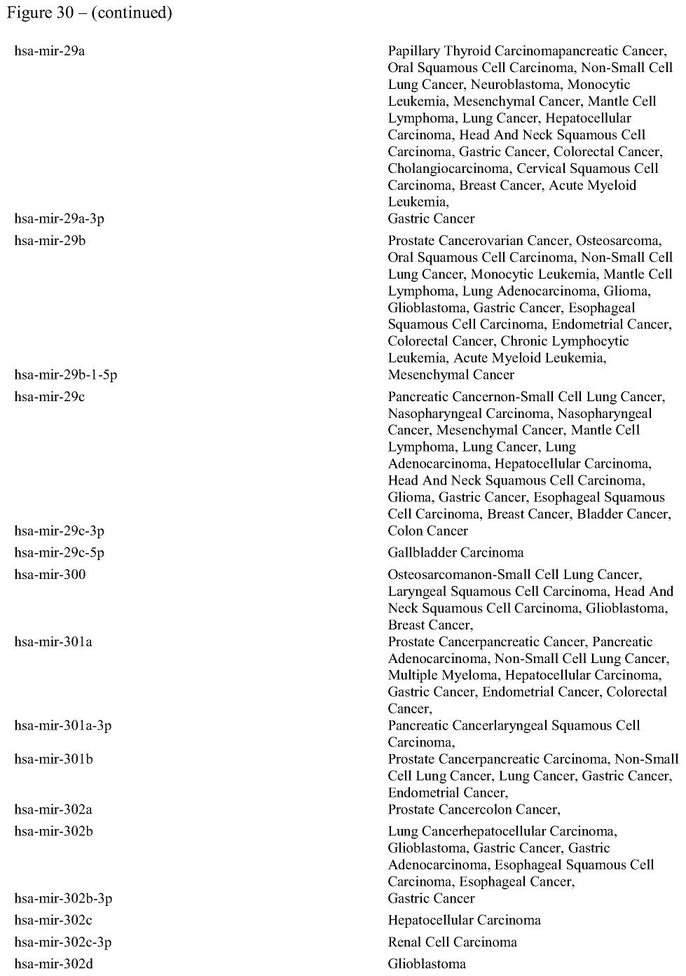

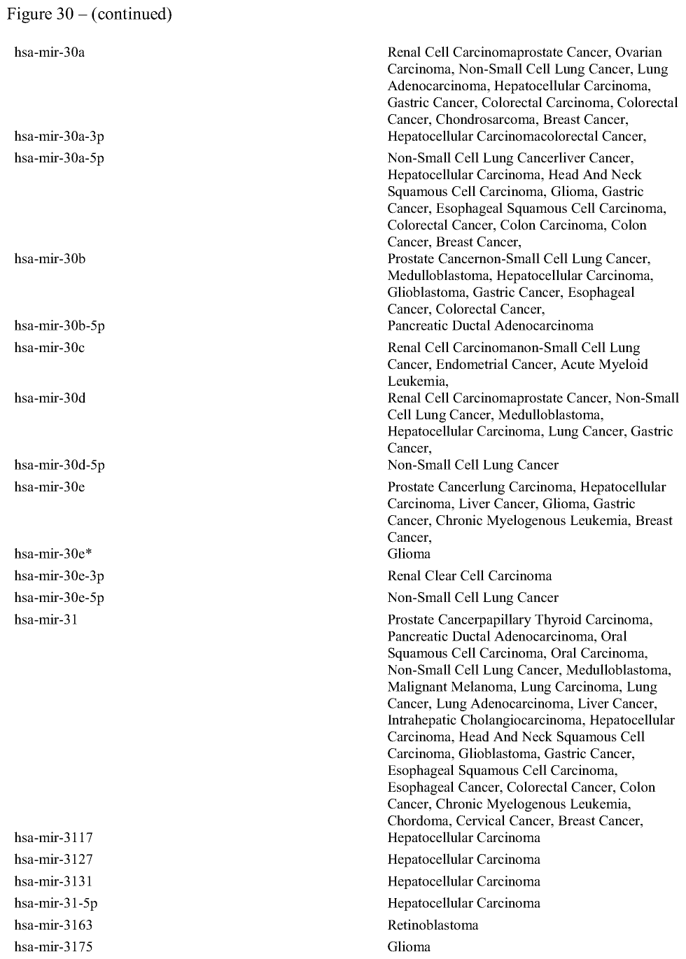

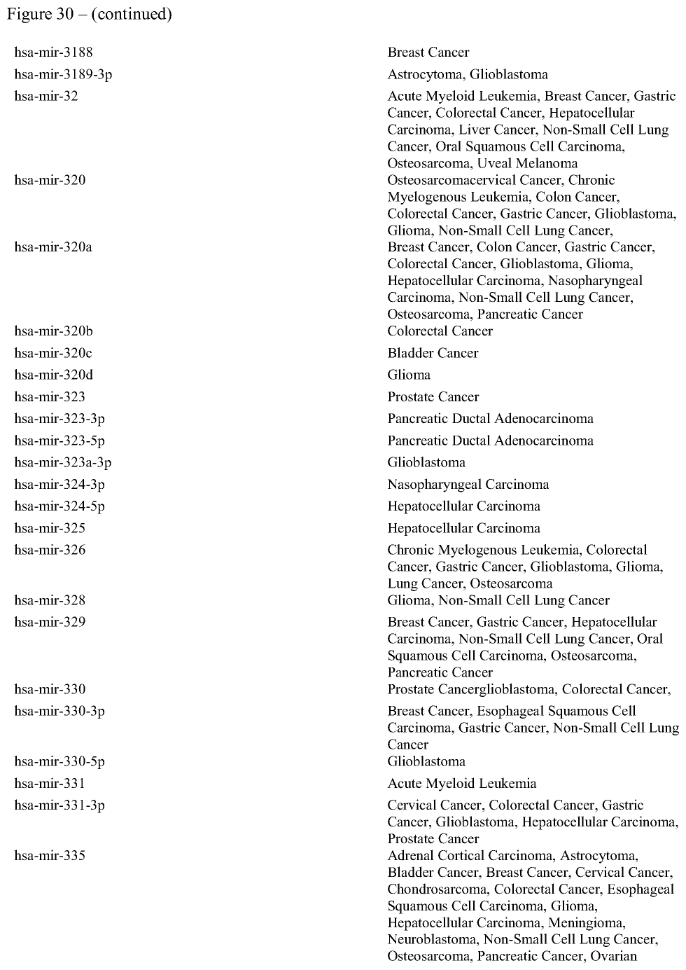

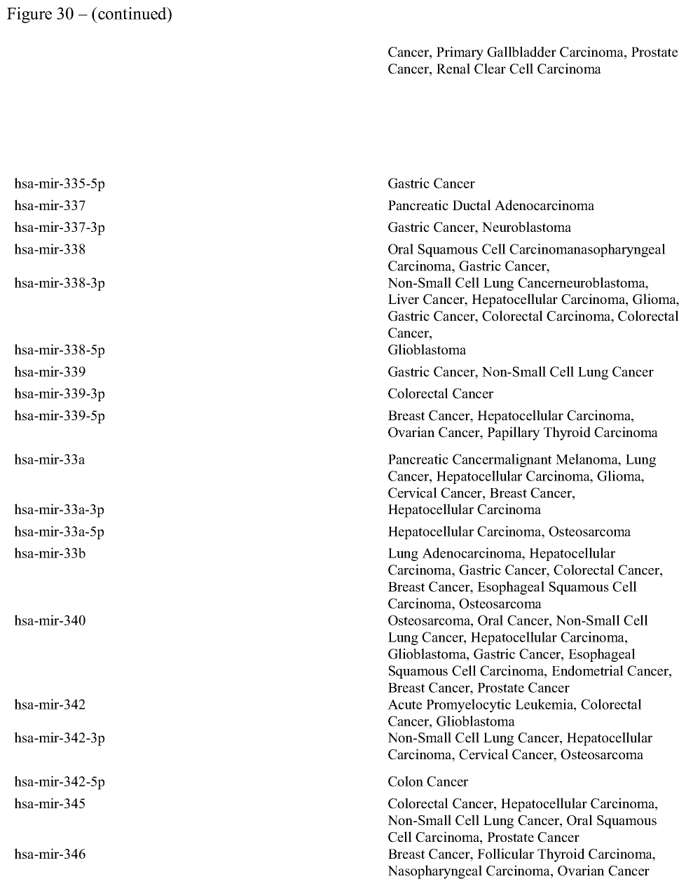

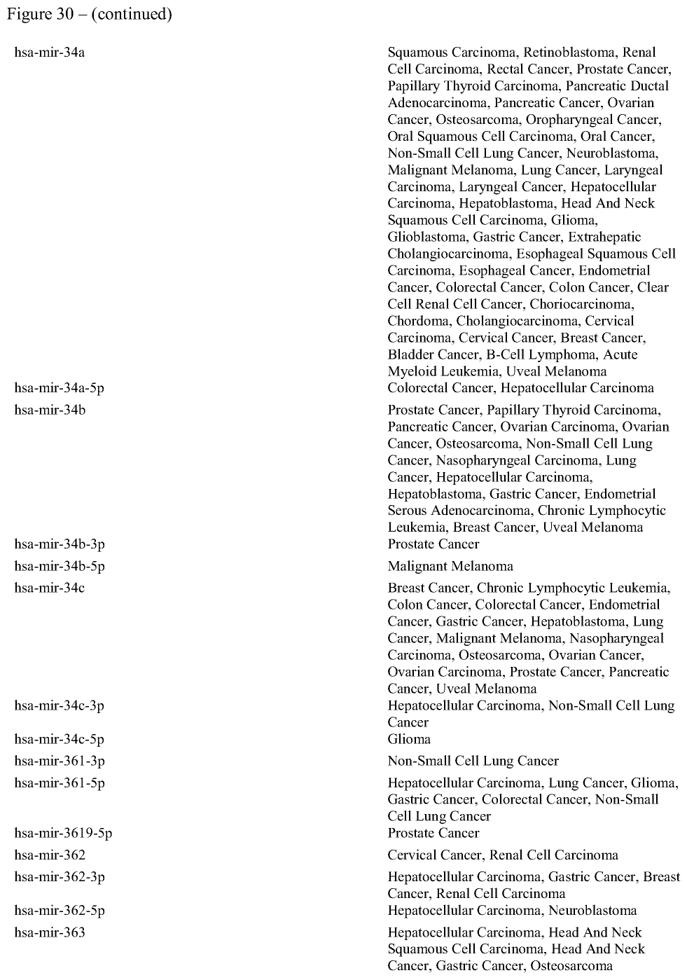

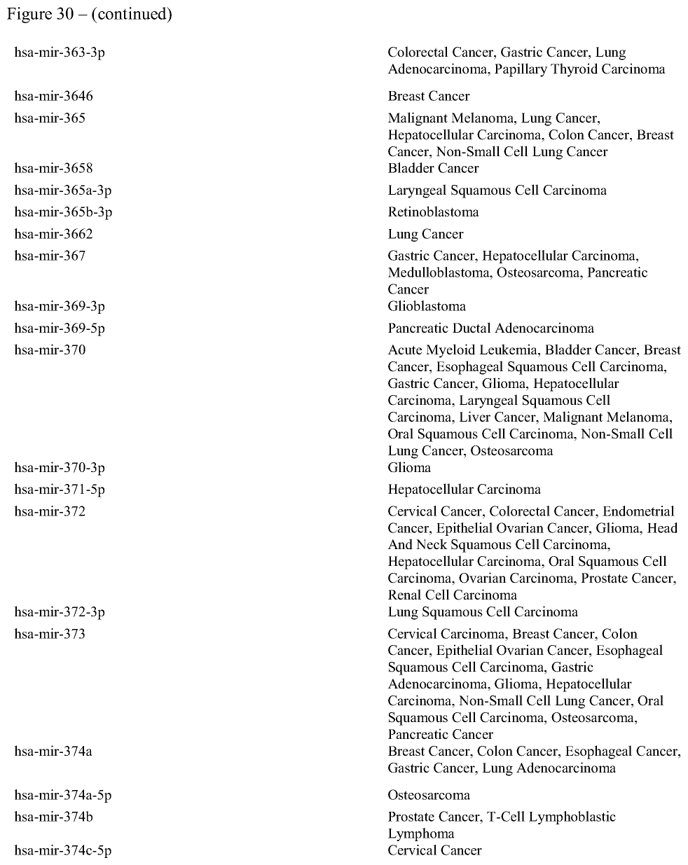

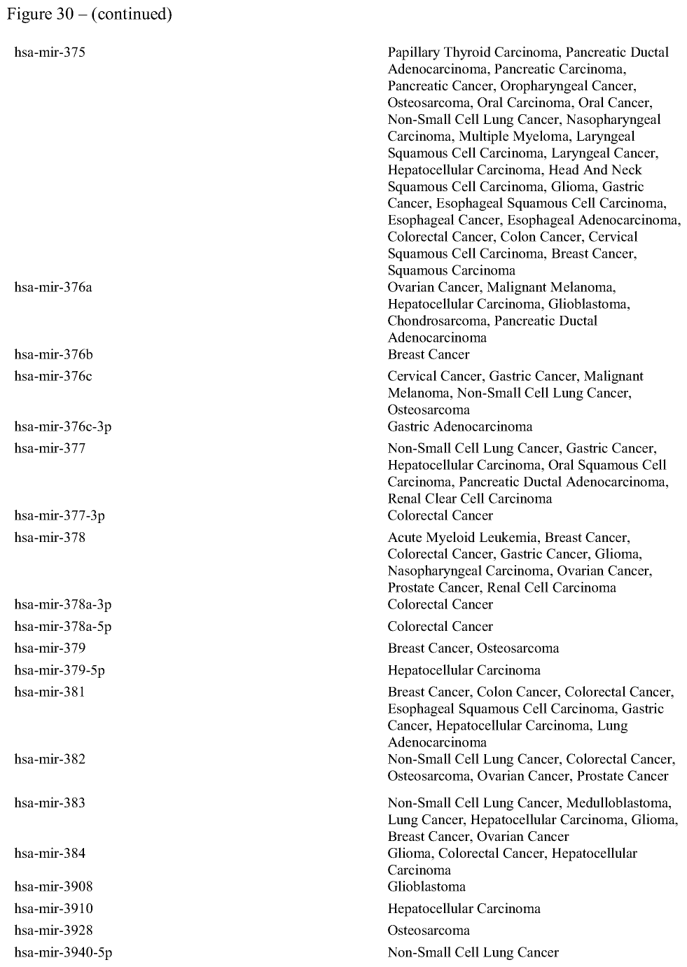

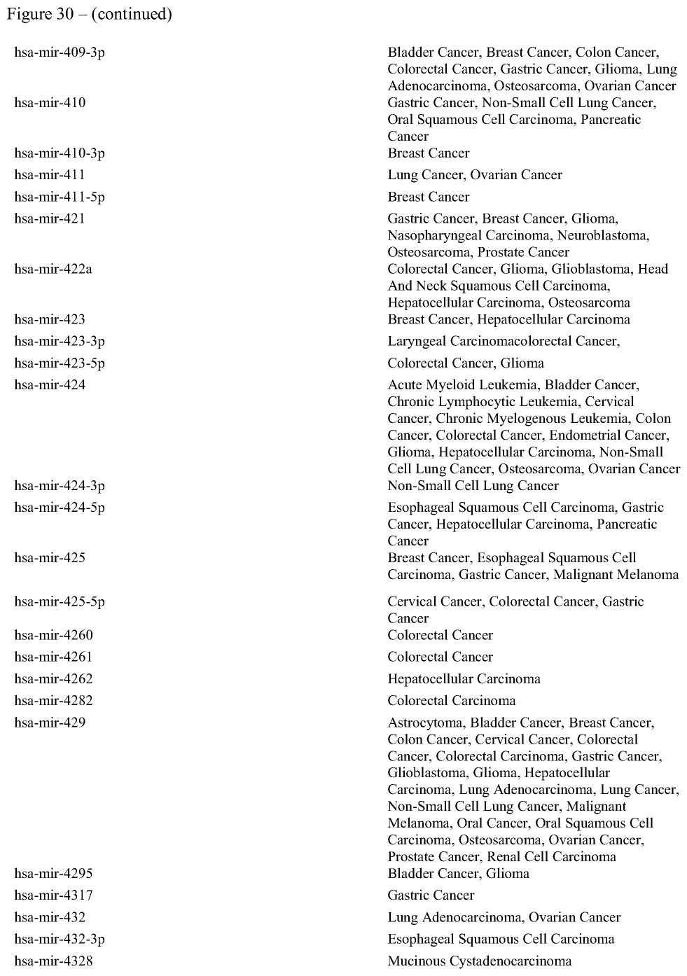

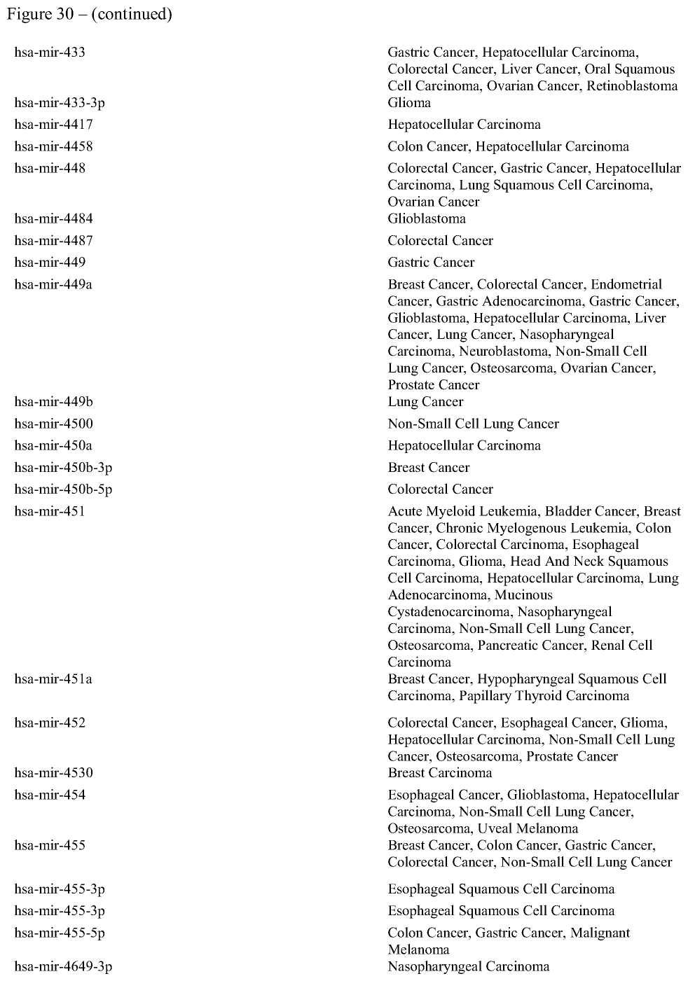

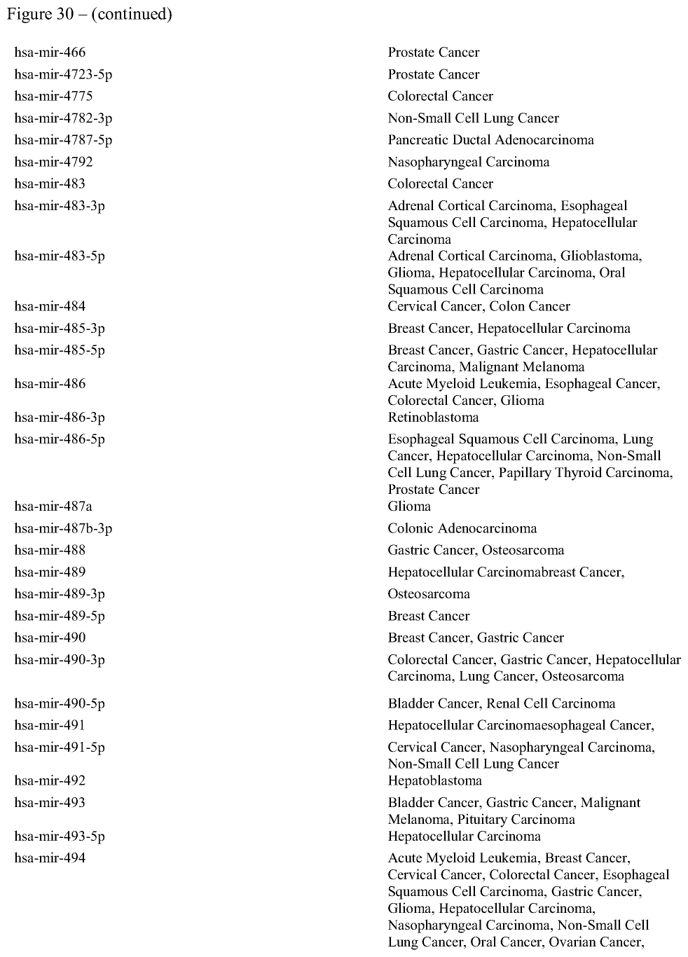

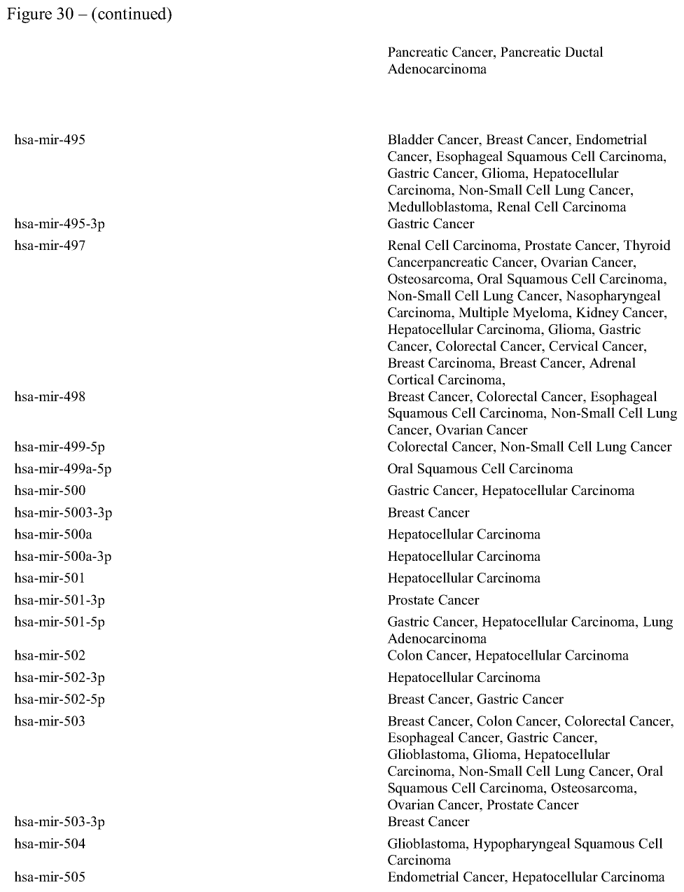

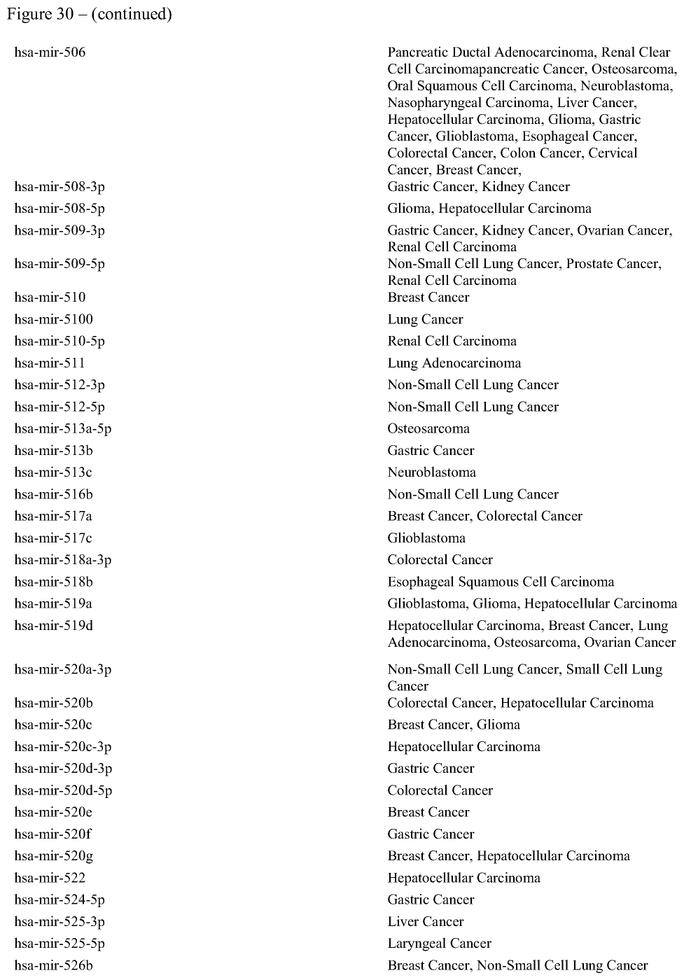

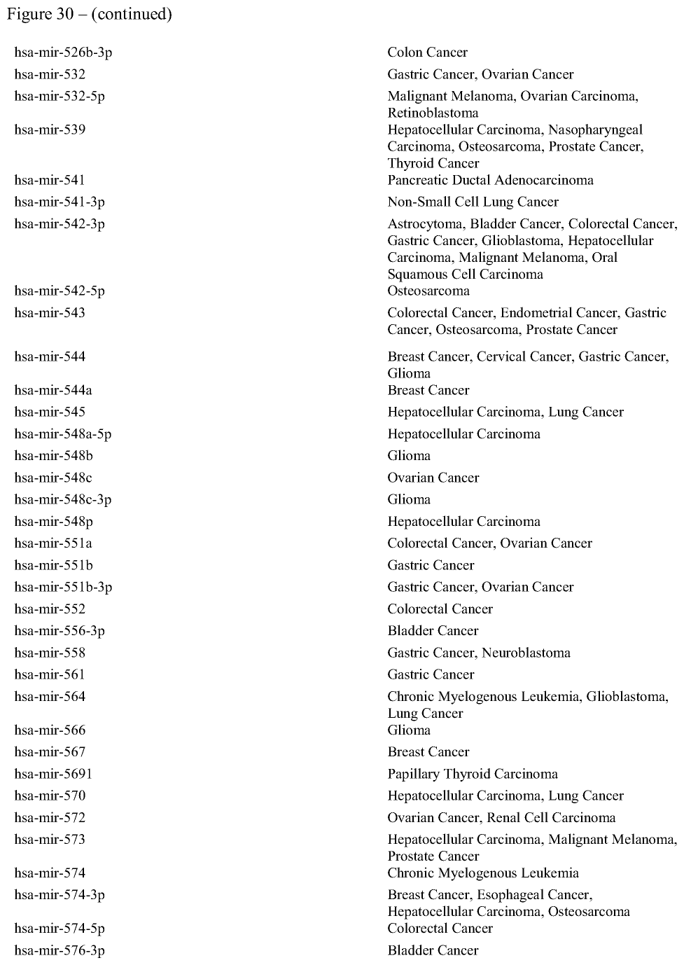

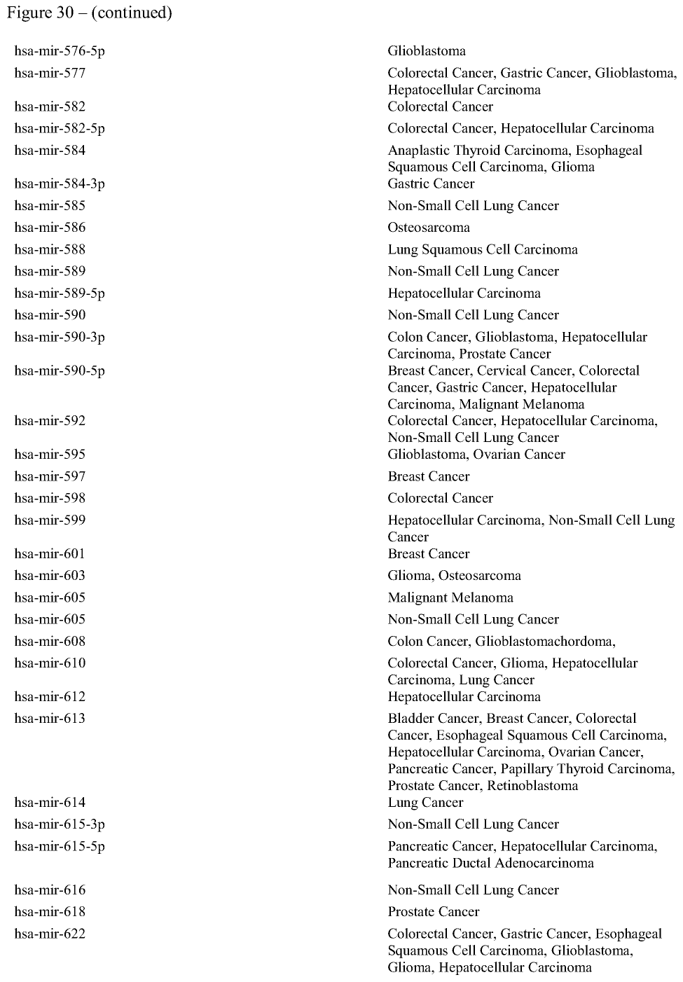

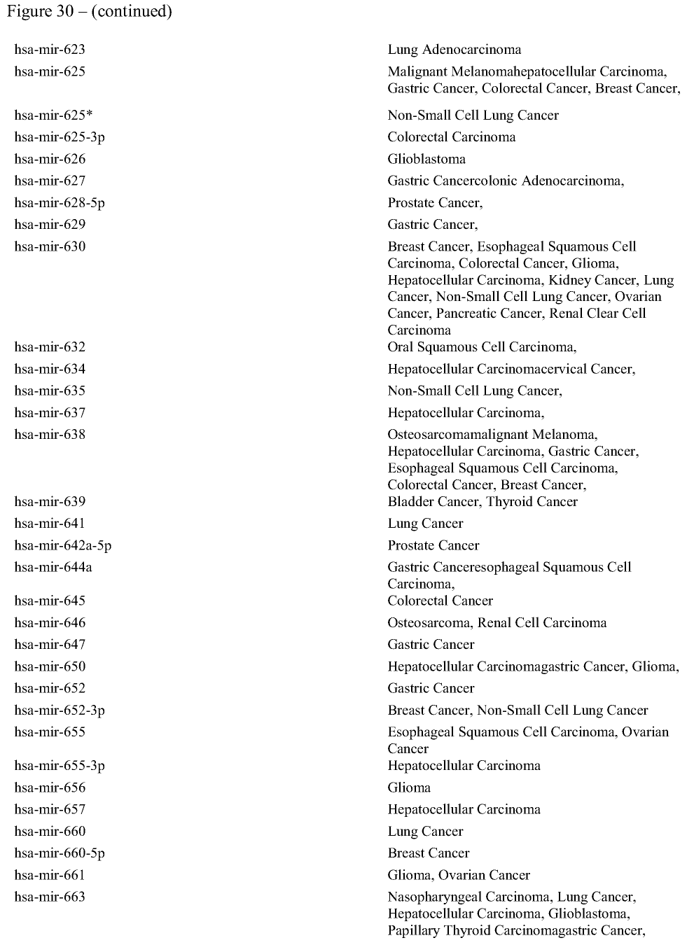

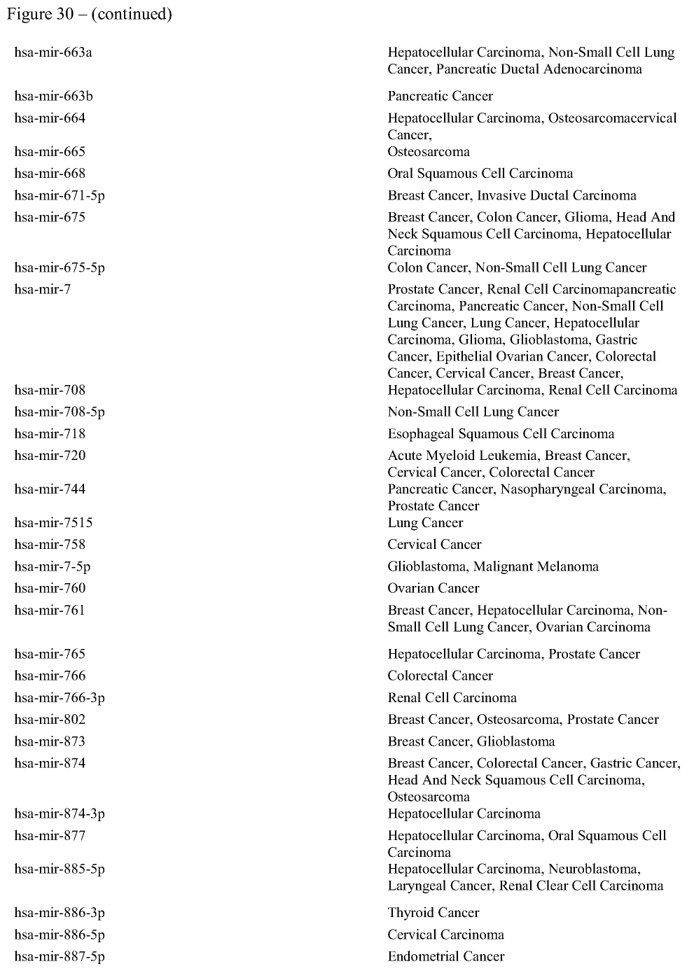

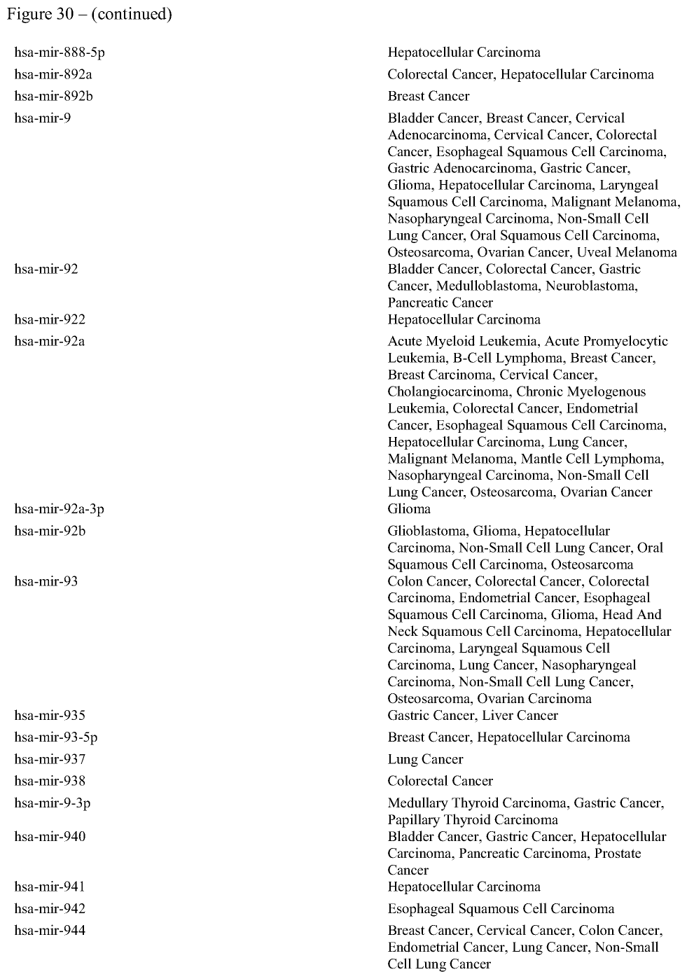

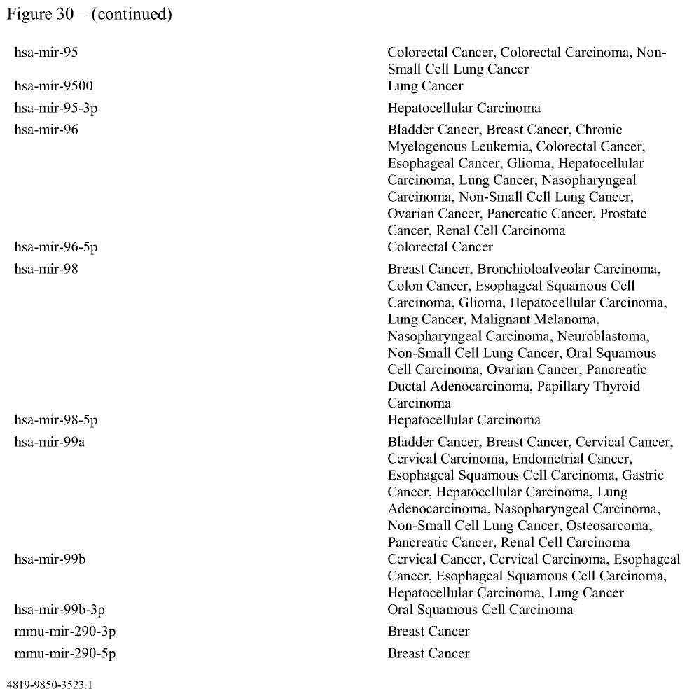

As described herein, microRNA is a small non-coding RNA molecule (containing about 19-22 nucleotides) which functions in RNA silencing and post-transcriptional regulation of gene expression. The human genome may encode over 2500 microRNAs, which are abundant in many mammalian cell types and appear to target at least 60% of the genes of humans and other mammals, as depicted in FIG. 2 (which also shows a histogram in which degradation of mature microRNA-21 provoked by an antisense agent specifically targeting microRNA-21 (“Antagomir-21”) is documented). As such, microRNAs represent a substantial class of targets for treating disease via modulation of gene expression. In particular, a role exists for microRNAs in fibrosis, where microRNA-21 is upregulated and microRNA-29 is downregulated in kidney, liver, and lung fibrosis. Additionally, a complex regulatory network of microRNAs is also involved in Parkinson's Disease. Suitable microRNA targets include microRNA-21 and the miRNAs recited in FIG. 30.

Exemplary disease targets for candidate lead compounds that bind to RNA oligonucleotides include fibrosis, cancer (e.g., via regulation of microRNAs and other non-coding RNAs) and other associated diseases and disorders. For example, such disease targets may include retinoblastoma, renal cell carcinoma, prostate cancer, papillary thyroid carcinoma, pancreatic ductal adenocarcinoma, pancreatic cancer, pancreatic adenocarcinoma, ovarian cancer, osteosarcoma, oropharyngeal cancer, oral squamous cell carcinoma, oral carcinoma, oral cancer, non-small cell lung cancer, neuroblastoma, nasopharyngeal carcinoma, multiple myeloma, mucinous cystadenocarcinoma, malignant melanoma, lung cancer, liver cancer, laryngeal squamous cell carcinoma, laryngeal carcinoma, laryngeal cancer, kidney cancer, hypopharyngeal squamous cell carcinoma, hepatocellular carcinoma, hepatoblastoma, head and neck squamous cell carcinoma, head and neck cancer, glioma, glioblastoma, gastrointestinal stromal tumor, gastric cancer, esophageal squamous cell carcinoma, esophageal cancer, endometrial cancer, diffuse large b-cell lymphoma, colorectal carcinoma, colorectal cancer, colon cancer, stomach cancer, chronic myelogenous leukemia, cholangiocarcinoma, cervical carcinoma, cervical cancer, breast cancer, b-cell lymphoma, adrenal cortical carcinoma and squamous carcinoma, all of which are associated with microRNA-21. Other disease targets contemplated for suitable miRNAs of the instant disclosure are recited for each miRNA in FIG. 30.

### Test Compound Library

Test compound libraries may be conveniently assembled from commercial sources and/or custom generated. For example, the libraries exemplified herein were curated by mining and identifying compounds from the literature and from internal fragment libraries. The compounds fell within the RO3 or RO5 (Lipinski definition), and the libraries were organized for high throughput screening.

### Cancer Cell Lines

In addition to the MCF-7 cell line exemplified herein, screening as described in the instant disclosure can be performed upon any one or many of known cancer cell lines, including, e.g., 600MPE, AU565, BT-20, BT-474, BT-483, BT-549, Evsa-T, Hs578T, MDA-MB-231, SkBr3, T-47D, CCF-STTG1, SW 1088, SW 1783, CHLA-02-ATRT, A172, U-138 MG, LN-18, LN-229, U-87 MG, U-118 MG, T98G, Hs 683, CHLA-01-MED, CHP-212, H4, D341 Med, Daoy, PFSK-1, DBTRG-05MG, M059K, M059J, IMR-32, BC3H1, bEnd.3, Neuro-2a, NB41A3, N1E-115, C6, C6/LacZ, 9L/lacZ, C6/lacZ7, F98EGFR, F98npEGFRvIII, F98, RG2, NCI-H1373, NCI-H1395, SK-LU-1, HCC2935, HCC4006, HCC827, NCI-H1581, NCI-H23, NCI-H522, NCI-H1435, NCI-H1563, NCI-H1651, NCI-H1734, NCI-H1793, NCI-H1838, NCI-H1975, NCI-H2073, NCI-H2085, NCI-H2228, NCI-H2342, NCI-H2347, NCI-H2066, NCI-H2286, NCI-H1703, NCI-H2135, NCI-H2172, NCI-H2444, NCI-H835, UMC-11, NCI-H720, A549, A-427, NCI-H596, SW 1573, NCI-H1688, NCI-H1417, NCI-H1672, NCI-H1836, HLF-a, NCI-H810, NCI-H292, NCI-H2126, DMS 79, DMS 53, DMS 114, SW 1271, NCI-H2227, NCI-H1963, SHP-77, NCI-H2170, NCI-H520, SW 900, NCI-H358, NCI-H727, LA-4, LL/2 (LLC1), KLN 205; DU-145, PC-3 and LNCaP, and other prostate cancer cell lines; RKO, RKO-AS45-1, HT-29, SW1417 [SW-1417], SW948 [SW-948], DLD-1, SW480 [SW-480], SW1116 [SW 1116, SW-1116], LS 174T, WiDr, COLO 320DM, COLO 320HSR [COLO 320 HSR], HCT-15, SW403 [SW-403], SW48 [SW-48], HCT-8 [HRT-18], HCT 116, LS123, LS 180, HX [HT1080 xeno], HP [HT1080 poly], CCD-18Co, CCD-33Co, CCD-112CoN, CCD 841 CoN, CCD 841 CoTr, FHC, Ramos.2G6.4C10, C2BBe1 [clone of Caco-2 (ATCC HTB-37)], RKO-E6, ATRFLOX [Mutatect], Hs 255.T, Hs 257.T, Hs 675.T, Caco-2, SK-CO-1, COLO 201, COLO 205, Hs 698.T, LoVo, T84, SW620 [SW-620], SNU-C1, XB-2, M-NFS-60, CT26.WT, CT26.CL25, CLT 85 [SKI 294/CLT 85], HT 29/36 [SKI 294/HT 29/36], HT 29/26 [SKI 294/HT 29/26], 1116NS-3d, PCA 31.1, PCA 33.28, 1116-NS-19-9, 7E12H12, CLT 152 [SKI 294/CLT 152], TAC-1, GPC-16, Detroit 562, FaDu, SCC-15, SCC-4, SCC-25, SCC-9, CAL 27, 006, 019, 029, Fc3Tg, FDC-1, 8A3B.6, 8B1B.1, 7D3A.2, BHFTE, FT, W6/32, S 1, FC3.Tg, Hs 157.Tg, EBTr (NBL-4), AGS, SNU-1, SNU-5, SNU-16, Hs 746T, NCI-N87 [N87], KATO III, HeLa DH, HR5-CL11, HtTA-1, HRS, X1/5, HeLa, C-41, C-4 II, HeLa S3, Ca Ski, HeLa229, Hep2 (HeLa derivative), HeLa B, Bu25 TK-, HeLa Ohio and HeLa (AC-free).

### Candidate Lead Compounds

Certain aspects of the disclosure relate to identification of the following candidate lead compounds as capable of binding microRNA-21 in an inhibitory manner.

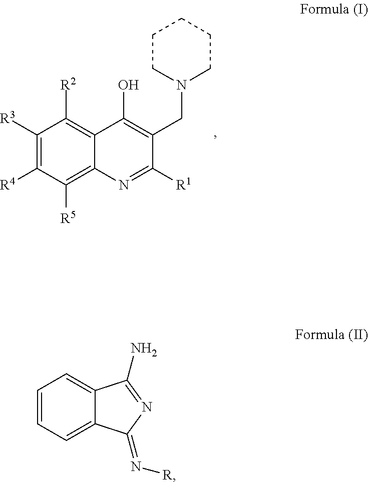

including Formula (1) selected from the following:

| Compound | R¹ | R² | R³ | R⁴ | R⁵ | 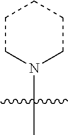 |
|---|---|---|---|---|---|---|
| BSI101534 | Ph | H | —OMe | H | H | 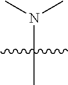 |
| BSI101536 | Ph | H | —OMe | H | H | 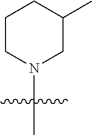 [SMILES: *N1CCCC(C)C1] |
| BSI101473 | —CH₃ | —OMe | H | H | —OMe | 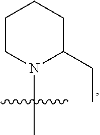 [SMILES: *N1CCCCC1CC] |

and/or Formula (II) selected from the following:

| Compound | R |
|---|---|
| BSI101484 | 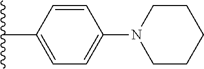 [SMILES: *c1ccc(N2CCCCC2)cc1] |
| BSI104171^{x} | H, |

or a salt thereof.

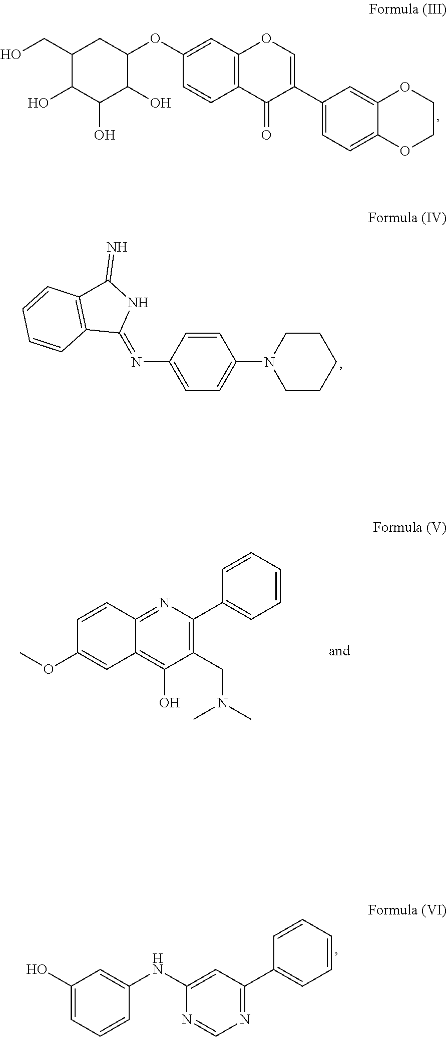 [SMILES: O=c1c(-c2ccc3c(c2)OCCO3)coc2cc(OC3CC(CO)C(O)C(O)C3O)ccc12 ; N=C1N/C(=N\c2ccc(N3CCCCC3)cc2)c2ccccc21 ; COc1ccc2nc(-c3ccccc3)c(CN(C)C)c(O)c2c1 ; Oc1cccc(Nc2cc(-c3ccccc3)ncn2)c1]

or a salt thereof.

Derivatization of the above compounds is also specifically contemplated.

### Pharmaceutical Compositions

In certain embodiments, the present disclosure provides for a pharmaceutical composition comprising a candidate compound of the present disclosure. A candidate compound can be suitably formulated and introduced into a subject and/or the environment of a cell by any means that allows for a sufficient portion of the compound to exert an effect in the subject or cell, if it is to occur. Many formulations for small molecules are known in the art and can be used.

For administration to a subject, the candidate lead compounds of the disclosure can be provided in pharmaceutically acceptable compositions. These pharmaceutically acceptable compositions comprise a therapeutically-effective amount of one or more of the candidate lead compounds, formulated together with one or more pharmaceutically acceptable carriers (additives) and/or diluents. As described in detail herein, the pharmaceutical compositions of the present disclosure can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C.sub.2-C.sub.12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of a compound, material, or composition comprising a compound described herein which is effective for producing some desired therapeutic effect in at least a sub-population of cells in an animal at a reasonable benefit/risk ratio applicable to any medical treatment. For example, an amount of a compound administered to a subject that is sufficient to produce a statistically significant, measurable decrease in the size of a tumor.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. Routes of administration suitable for the methods of the disclosure include both local and systemic administration. Generally, local administration results in more of the administered candidate lead compound being delivered to a specific location as compared to the entire body of the subject, whereas, systemic administration results in delivery of the candidate lead compound to essentially the entire body of the subject. One method of local administration is by intramuscular injection.

In the context of administering a compound treated cell, the term “administering” also include transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species).

Furthermore, the candidate lead compounds can be formulated in the form of ointments, creams powders, or other formulations suitable for topical formulations. Such formulations can comprise one or more agents that enhance penetration of active ingredient through skin. For topical applications, the candidate lead compound can be included in wound dressings and/or skin coating compositions.

A candidate lead compound or composition comprising same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In preferred embodiments of the aspects described herein, the compositions are administered by intravenous infusion or injection.

A compound described herein can be co-administrated to a subject in combination with a pharmaceutically active agent. Exemplary pharmaceutically active compound include, but are not limited to, those found in Harrison's Principles of Internal Medicine, 13.sup.th Edition, Eds. T. R. Harrison et al. McGraw-Hill N.Y., N.Y.; Physicians' Desk Reference, 50.sup.th Edition, 1997, Oradell N.J., Medical Economics Co.; Pharmacological Basis of Therapeutics, 8.sup.th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

The candidate lead compound and the pharmaceutically active agent can be administrated to the subject in the same pharmaceutical composition or in different pharmaceutical compositions (at the same time or at different times). When administrated at different times, the candidate lead compound and the pharmaceutically active agent can be administered within 5 minutes, 10 minutes, 20 minutes, 60 minutes, 2 hours, 3 hours, 4, hours, 8 hours, 12 hours, 24 hours of administration of the other. When the candidate lead compound and the pharmaceutically active agent are administered in different pharmaceutical compositions, routes of administration can be different.

The amount of the candidate lead compound that can be combined with a carrier material to produce a single dosage form will generally be that amount of the candidate lead compound that produces a therapeutic effect. Generally, out of one hundred percent, this amount will range from about 0.01% to 99% of the compound, preferably from about 5% to about 70%, most preferably from 10% to about 30%.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

As used herein, the term ED denotes effective dose and is used in connection with animal models. The term EC denotes effective concentration and is used in connection with in vitro models.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that the candidate lead compound is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 μmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this disclosure, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In some embodiments, the compositions are administered at a dosage so that the candidate lead compound or a metabolite thereof has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered every day or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

The pharmaceutical compositions can be included in a kit, container, pack, or dispenser together with instructions for administration.

### Methods of Treatment

The present disclosure provides for both prophylactic and therapeutic methods of treating a subject at risk of (or susceptible to) a disease or disorder.

In certain aspects, the disclosure provides a method for preventing in a subject, a disease or disorder as described herein, by administering to the subject a therapeutic agent (e.g., a candidate compound as described herein). Subjects at risk for the disease can be identified by, for example, one or a combination of diagnostic or prognostic assays known in the art. Administration of a prophylactic agent can occur prior to the detection of, e.g., a disease or disorder in a subject, or the manifestation of symptoms characteristic of the disease or disorder, such that the disease or disorder is prevented or, alternatively, delayed in its progression.

Another aspect of the disclosure pertains to methods of treating subjects therapeutically, i.e., altering the onset of symptoms of the disease or disorder. These methods can be performed in vitro (e.g., by culturing the cell with a candidate compound) or, alternatively, in vivo (e.g., by administering a candidate compound to a subject).

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

This disclosure is further illustrated by the following examples which should not be construed as limiting.

### EXAMPLES

### Example 1

### Role of Aberrant microRNA Expression in Disease

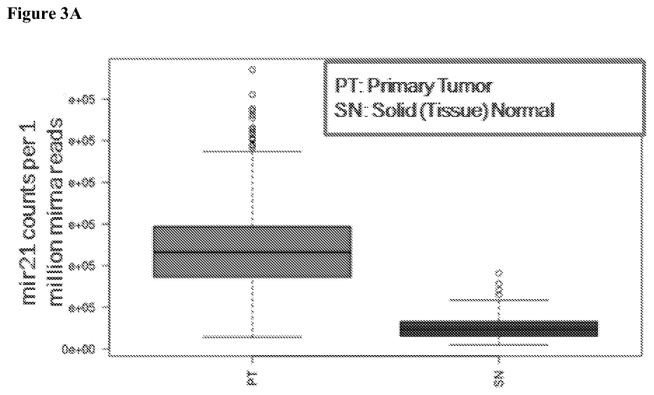

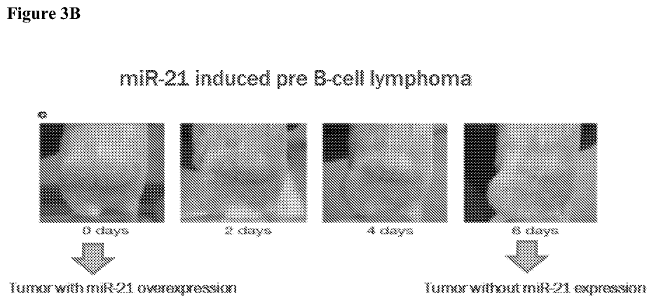

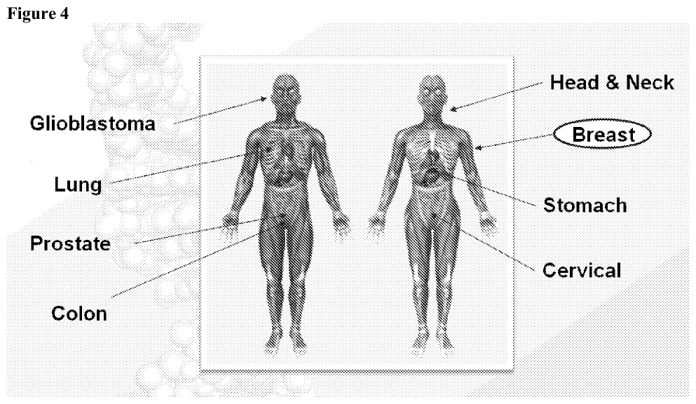

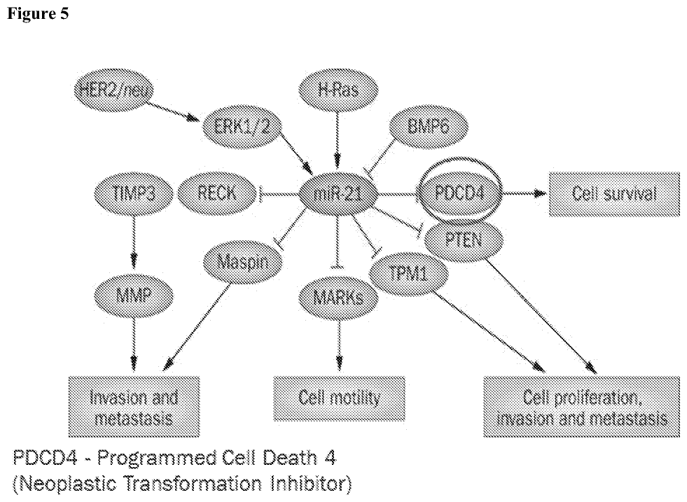

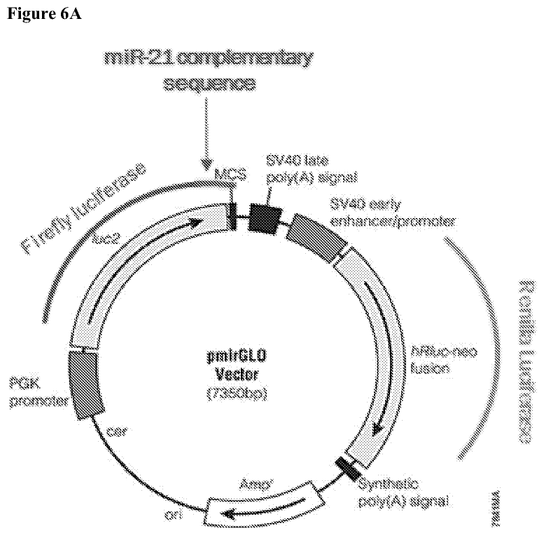

Aberrant expression of a number of microRNAs has been shown to be involved in the development of a number of cancers. In particular, as shown in FIG. 4, microRNA (e.g., microRNA-21) overexpression has been detected in cancers including: glioblastoma, breast cancer, lung cancer, prostate cancer, stomach cancer, colon cancer, cervical cancer, and head and neck cancer. As FIG. 5 demonstrates, microRNA-21 plays a role in tumor suppression via inhibition of RECK, Maspin, MARKs, TPM1, PTEN, and PDCD4 (Programmed Cell Death 4, a neoplastic transformation inhibitor). microRNA-21 profiles for all stages of breast cancer compared to normal solid tissue are shown in FIG. 3A. microRNA-21 counts per 1 million microRNA reads were plotted against primary tumor and solid normal tissue. The dark line in each box represents the median. FIG. 3B demonstrates expression of microRNA-21 in B-cell lymphoma, with microRNA-21 initially overexpressed at day 0, yet then inactivated via Cre and Tet-off technologies (Medina et al. Nature 467: 86-90), resulting in reduced microRNA-21 expression, which in turn produced shrinkage of B-cell lymphoma tumors at days 2-6, as shown.

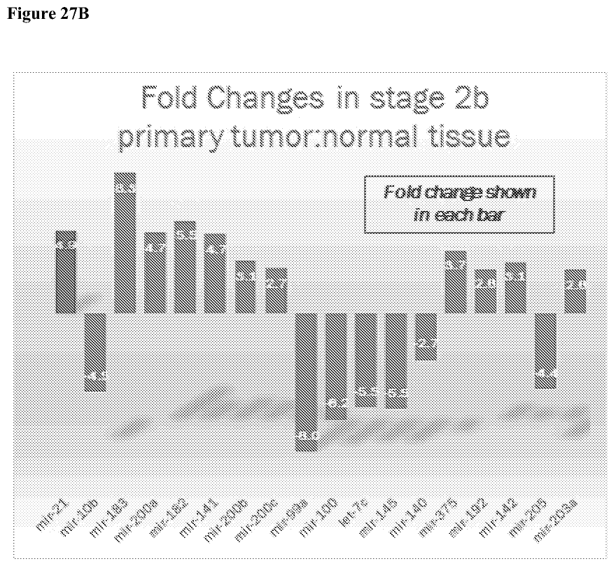

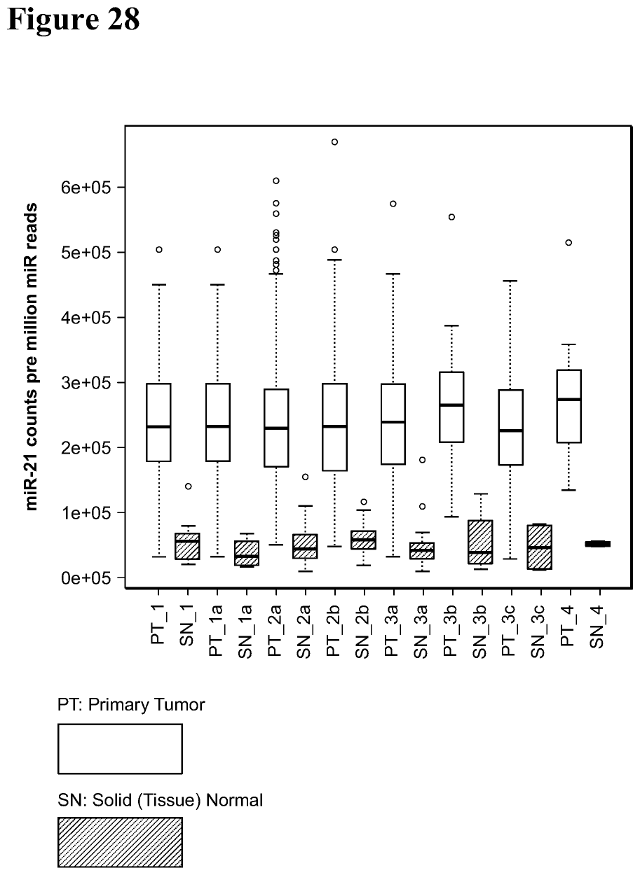

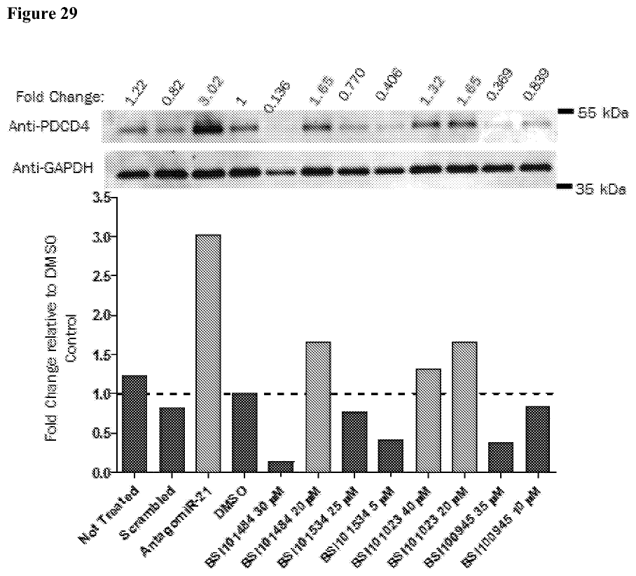

Various microRNAs have been identified as overexpressed in breast cancer. FIGS. 27A and 27B depict graphs demonstrating differentially expressed microRNAs in Stage 2b breast cancer patients. FIG. 27A depicts the median microRNA counts per million microRNA reads in stage 2b primary breast tumors. FIG. 27B depicts the fold changes in stage 2b primary tumor compared to normal tissue for various microRNAs. Differential expression of the microRNAs shown were statistically significant with adjusted p-values <0.001. All fold changes shown were greater than 2.5 or less than -2.5. FIG. 28 depicts a graph demonstrating microRNA-21 expression profile in breast cancer by stage (TCGA). MCF-7 breast cancer cells were grown in the presence of insulin. The expression of 871 microRNAs was examined via microarrays. microRNA-21, let-7f, and let-7a were especially overexpressed in breast cancer cells, exhibiting a microarray intensity signal of over 20,000. These results further underscored the role of microRNA-21 overexpression in breast cancer.

Thus, microRNA-21 has been observed to be overexpressed in different cancers (e.g., breast and blood cancer). In addition, microRNAs (e.g., microRNA-21) represent not just a biomarker for disease but also a target for treatment.

### Example 2

### Primary Screening of Compounds Using NMR and Biological Assays

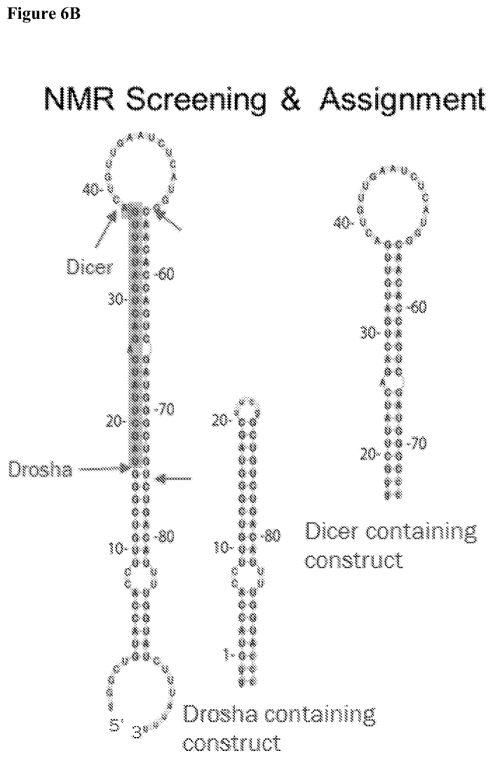

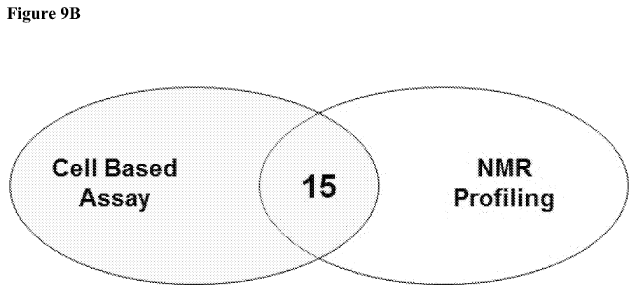

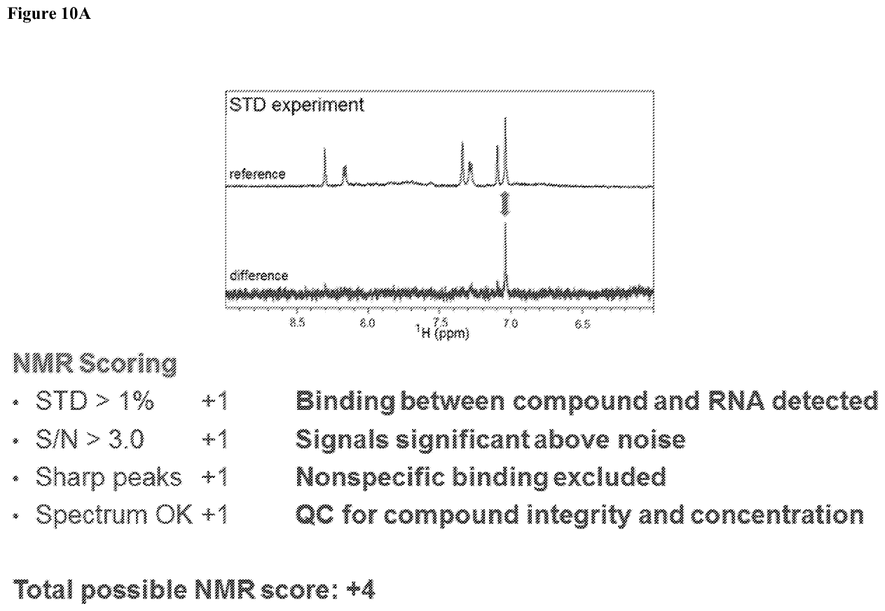

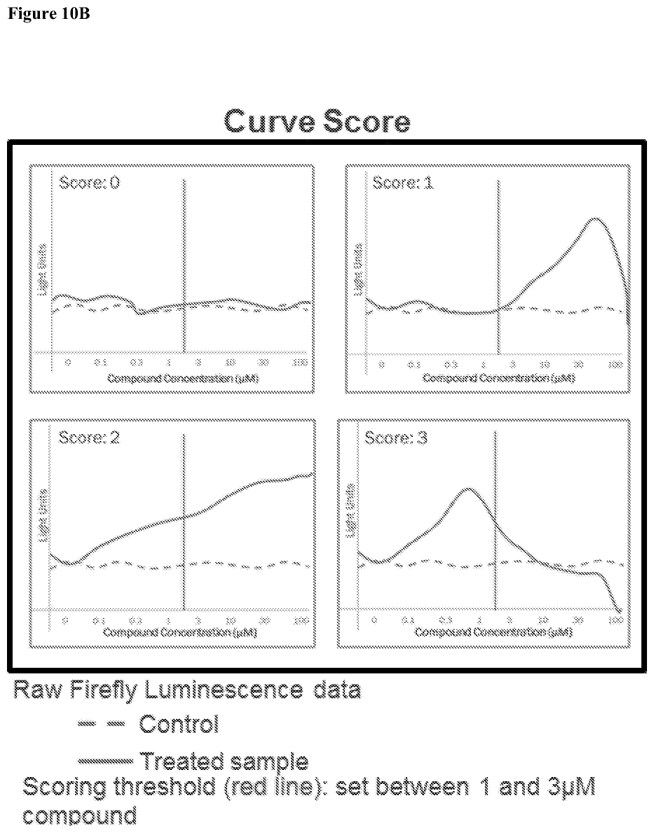

NMR was used to screen a library of compounds for microRNA binding, as a form of testing performed in parallel with the biological reporter assay screening methods described herein. FIGS. 9A and 9B depict the NMR approach to primary screening of compounds targeting RNA molecules. FIG. 9A shows a diagram depicting an NMR spectrum from a small molecule library screen and a classification of the corresponding hits. A small molecule library of 696 compounds was assembled and screened to identify agents possessing the ability to bind Dicer cleavage site sequence-containing and/or Drosha cleavage site sequence-containing microRNA constructs. FIG. 6B depicts microRNA stem loop structures and the location of sequences within the RNA structure that are targeted by Dicer and Drosha, respectively. Compounds bound to these structures were detected by NMR. Using NMR, peaks indicative of compounds binding to Dicer and/or Drosha constructs were recorded as positive NMR assay hits. FIG. 10A shows a sample NMR spectrum depicting peaks and how such chromatograms were translated into an NMR assay score. A peak selectively appearing in compound-treated assays (as compared to DMSO or other control assays) at greater than 1% of the reference spectrum indicated that binding between compound and RNA was detected. Such a peak was assigned a score of +1. A signal to noise ratio of greater than 3.0 indicated signal above noise was significant. Such a result was also assigned a score of +1 in the NMR assay. Sharp peaks indicated nonspecific binding was excluded; this also was assigned a score of +1 in the NMR assay. Finally, the spectrum was compared against the spectrum predicted for the chemical structure and concentration. Agreement suggests good chemical integrity and solubility and earned an additional score of +1. Based on these parameters, a screened compound has a possible NMR score of 0-4.

Dicer processing of the RNA structure comprising a Dicer cleavage site used in NMR experiments was also confirmed. RNA was transcribed and end-labelled in preparation for gel purification. RNA was in vitro transcribed and subjected to treatment with Dicer. hrDicer (human recombinant Dicer) was confirmed to process such RNAs (data not shown). hrDicer was specifically confirmed to generate products having the expected size of mature microRNA-21.

As described herein, subsets of tested compounds were classified according whether they bind a Dicer cleavage site-containing construct, a Drosha cleavage site-containing construct, or both constructs. Of the positive hits identified in this assay, 66 compounds bound Dicer cleavage site-containing microRNA constructs, 5 compounds bound Drosha cleavage site-containing microRNA constructs, and 14 compounds were shown to bind to both Dicer cleavage site containing- and Drosha cleavage site-containing microRNA constructs.

In parallel with the above-described NMR binding assays, cell-based reporter assays were also performed upon the 696-compound library. In particular, reporter constructs were transfected into MCF-7 breast cancer cell lines, which were then used to screen compounds for microRNA inhibition. FIG. 6A depicts a diagram of the reporter gene construct, pmirGLO vector, that was used in the cell based assay. The construct included a microRNA-21 complementary sequence as well as reporter sequences for firefly luciferase and renilla luciferase. Optionally, the expression cassette could be designed to include sequences complementary to microRNAs other than microRNA-21 (the one exemplified herein). Administration of antagomiR-21 acted a positive control in such assays, turning on firefly luciferase expression in the reporter assay as a result of its sequestration/degradation of mature microRNA-21, which would otherwise exert a silencing effect upon the microRNA-21 complementary sequence of the reporter construct. An IncuCyte ZOOM System was used to perform the cell-based assays described herein.

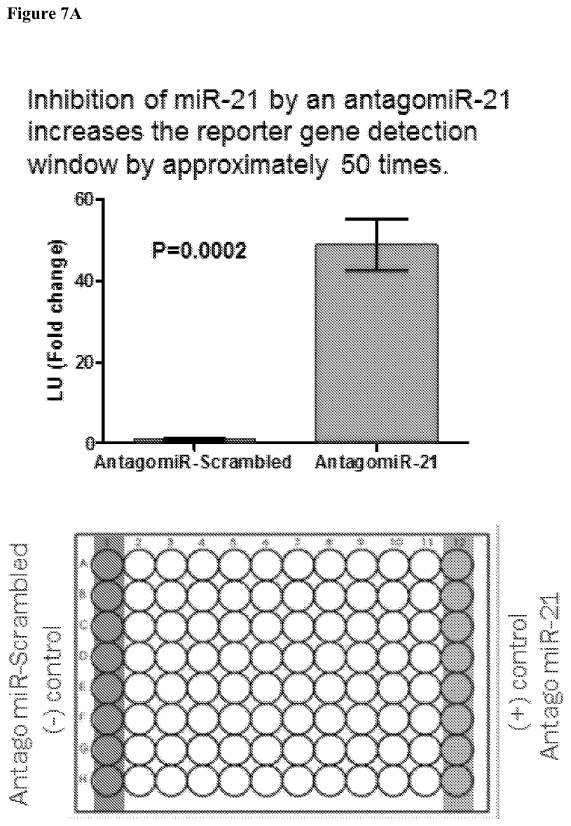

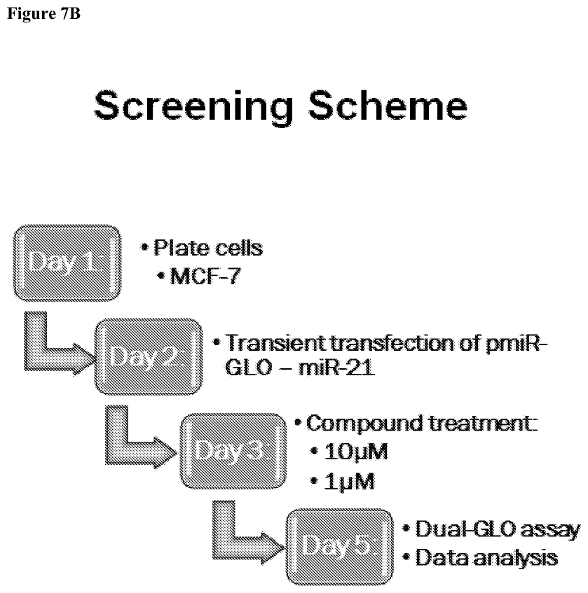

As described herein and summarized in FIG. 7B, MCF-7 breast cancer cells were plated on Day 1. On Day 2, reporter constructs containing a complementary sequence to microRNA-21 (pmicroRNA-GLO) were transiently transfected into the MCF-7 cell line. Transfected cells were treated with compounds at concentrations of 1 μM and 10 μM. On Day 5, the Dual-GLO reporter assay was performed and data was analyzed. The reporter enzymes (renilla and firefly luciferase) were expressed and assessed by 48 hours post-transfection (the Day 5 mark). Fold change in luciferase units were plotted against antagomiR-21 (positive control) and a scrambled version of antagomiR-21 (negative control). As shown in FIG. 7A, this example demonstrated that inhibition of microRNA-21 by an antagomiR-21 construct increased expression of the reporter gene (firefly luciferase, normalized to renilla luciferase as a transfection efficiency control) by approximately 50 times. A scrambled antagomiR-21 sequence did not produce expression of the reporter gene.

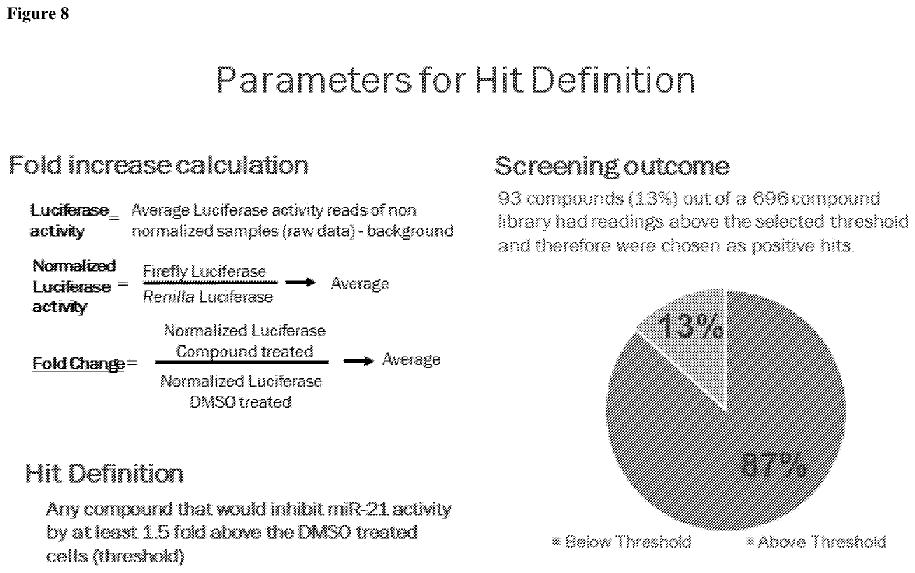

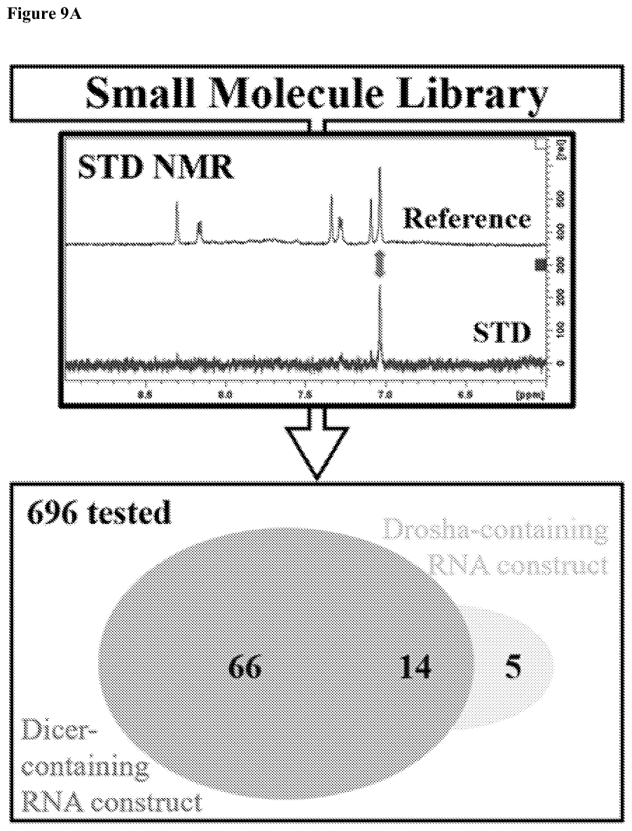

Candidate compound hits in the reporter assay were identified as any compound that inhibited microRNA activity (e.g., microRNA-21 activity) in cell culture by at least 1.5-fold above cells treated with DMSO alone, as depicted in FIG. 8. (Optionally, the threshold at which candidate compound hits are identified in the reporter assay can be established as at least 1.25-fold, at least 1.75-fold, at least 2-fold, at least 2.5-fold, at least 3-fold, etc.) Fold change values were determined by comparing total average normalized luciferase activity of compound treated cells to normalized luciferase activity of DMSO treated cells. As described herein, 93 compounds (13%) of the 696 compound library produced readings above threshold in the reporter assay and were selected as positive hits.

In the reporter assay, compounds were assigned a score based upon the shape of curves of light unit intensity observed at different compound concentrations, as compared to a control curve, as shown in FIG. 10B. Light units were measured based on raw firefly luminescence data. FIG. 10C depicts a graph demonstrating the potency score also used in the reporter assay screen. Luciferase was normalized to Renilla and the fold-change was determined by comparing normalized compound samples relative to DMSO control samples. As described herein, compounds were assigned a score based upon identifying the lowest concentration at which a 2 fold or higher change in activity was achieved. More potent compounds receive a higher score in this phase of the reporter assay, while less potent compounds were scored lower, on a potency scale of 0 to 7. As demonstrated in FIGS. 10B and 10C, there was a bias towards selecting compounds that possessed higher activity at lower concentrations. For scoring, a maximum 14 point scale scoring system was employed, based upon 4 possible points awarded from NMR screens and 10 possible points awarded from the reporter gene assay, based on the dose response of such compounds (with curve shape producing a score of between 0 and 3, and potency score ranging from 0 to 7). Hits were classified according to their performance in both the cell-based assays and in NMR profiling. As depicted in FIG. 9B, 15 hits were identified for further analysis by aggregating the cell-based assays and NMR profiling screens. FIG. 11 depicts a list of screened compounds identified as positive hits under the parallel screening methods described herein. The molecular weight, NMR score, functional score, total score, IC₅₀ and IC₂₅ values obtained are listed for compounds BSI101023, BSI101484, BSI101534, and BSI100945. BSI101023, BSI101484, BSI101534, and BSI100945 have total combined NMR and cell-based scores of 13, 10, 10, and 9 respectively.

qPCR methods were also employed in screening for test compounds that bind small RNAs. A TaqMan strategy was employed for identifying compounds that bind mature microRNA-21. snU6 was used as a loading control in such assays. A SyberGreen detection strategy was employed for identifying compounds that bind pre-microRNA-21. snU6 was also used as a loading control in these assays. As described herein, qPCR strategies have also been performed upon long RNAs, to examine the pri-microRNA-21 form.

### Example 3

### Additional Screening of Compounds Using NMR and Biological Assays

Subsets of the compounds regarded as positive hits under both NMR and cell-based assays were further examined for their interactions with microRNAs. FIG. 13A depicts the reporter assay results for hit compound BSI101023, including graphs of raw luciferase activity and the fold change relative to DMSO at different concentrations. BSI101023 was analyzed in a proliferation assay where the percent cell confluency of MCF-7 was measured in the presence of BSI101023 (at 3 μM, 10 μM, 30 μM, and 100 μM), as compared to a DMSO (at 0.5%) control. As demonstrated in FIG. 13B, cell growth declines in a dose-dependent manner with increasing concentration of the test compound BSI101023.

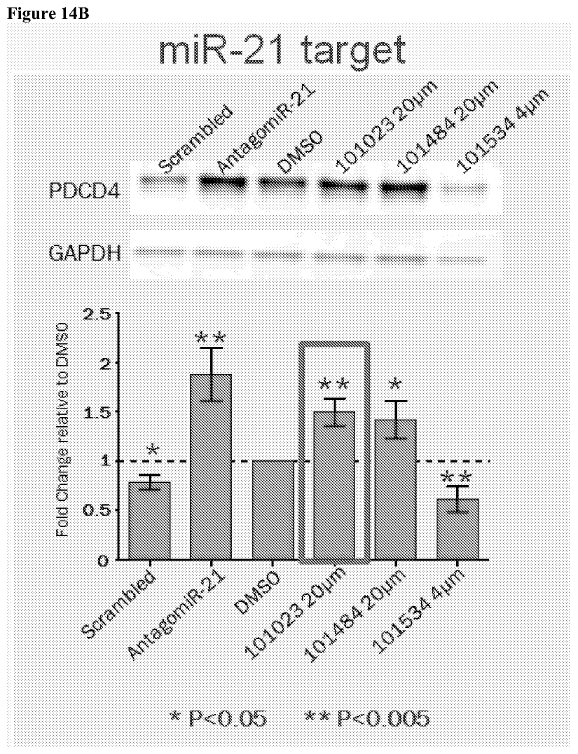

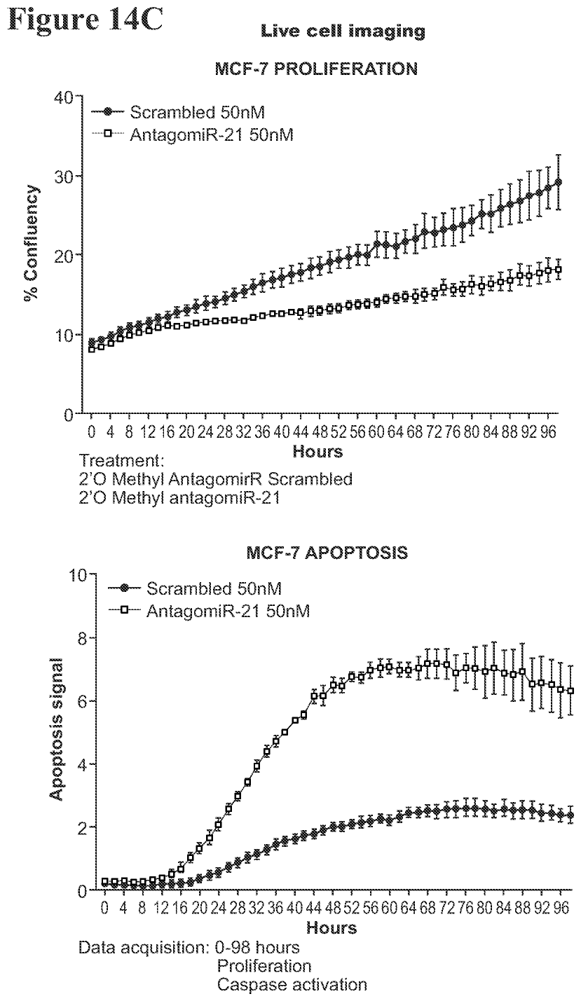

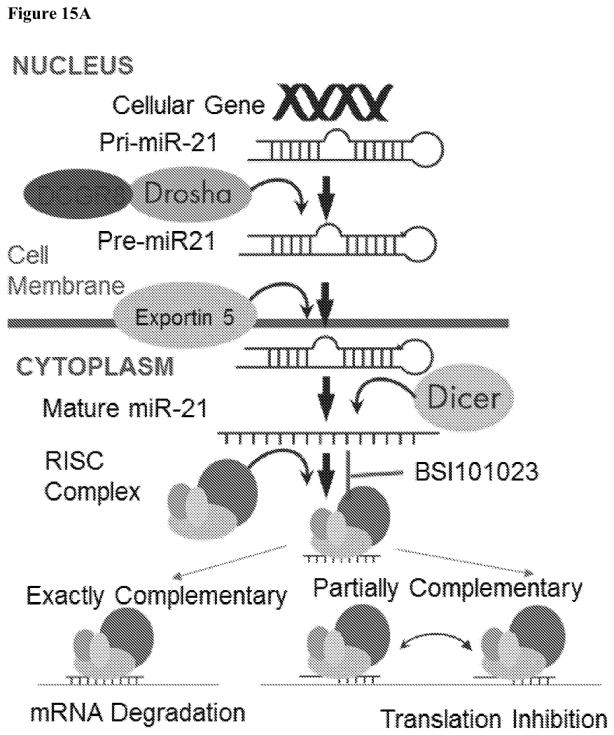

Scratch assays were used to analyze the functionality of certain test compounds identified as microRNA-binding. BSI101023 was analyzed for the ability to block re-population of cells at different compound concentrations, as compared to a DMSO-treated control, as depicted in FIG. 14A. Relative wound density was measured for DMSO (at 0.5%) and BSI101023 (at 12.5 μM, 25μM, 50 μM, and 100 μM). It was thereby demonstrated that BSI101023 inhibited cell migration, consistent with its abrogation of microRNA-21 activity. FIG. 14B depicts the downstream effects of microRNA-21 antagonism when hit compounds BSI101023, BSI101534, and BSI101484 were administered. The fold change in Programmed Cell Death 4 (PDCD4) levels—a downstream molecule in the microRNA-21 pathway—was examined (using GAPDH as an internal control) relative to DMSO treatment in scrambled antagomiR-21 samples, samples treated with the antagomiR-21 oligonucleotide agent, and in samples treated with BSI101023 (20 uM), BSI101484 (20 uM), and BSI101534 (4 uM), with results plotted in histogram format. BSI101023 and BSI101484 both significantly increased expression of PDCD 4, a downstream effector of microRNA-21, and also induced expression of accumulation of the mature species. For statistical analyses, * is P<0.05 and ** is P<0.005. Both results paralleled those observed for the positive control antagomiR-21 oligonucleotide agent. As described elsewhere herein, BSI101023 appears to inhibit loading of mature microRNA-21 into the RISC complex, while BSI101484 has been projected to bind to the loop present in the pri-microRNA-21, apparently leading to an early stage degradation of pri-microRNA-21.

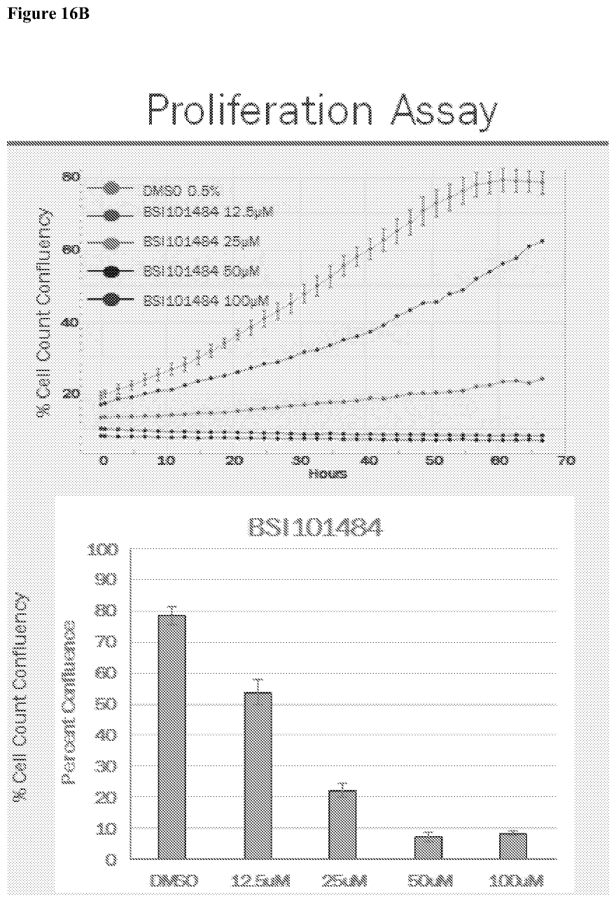

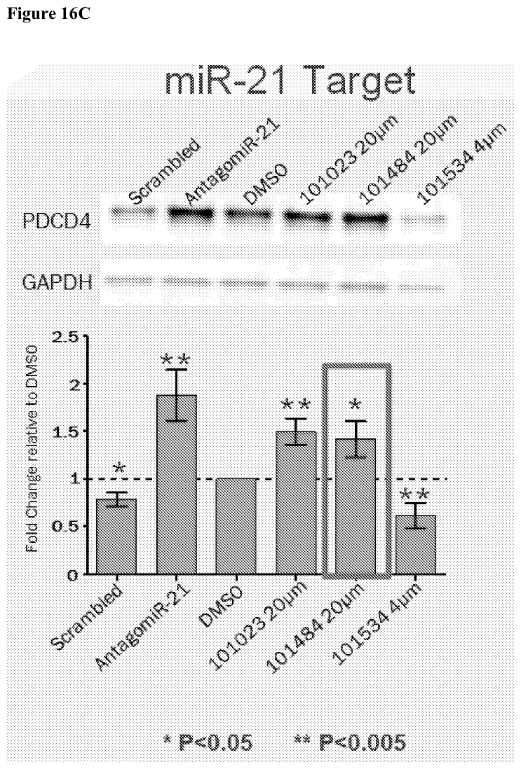

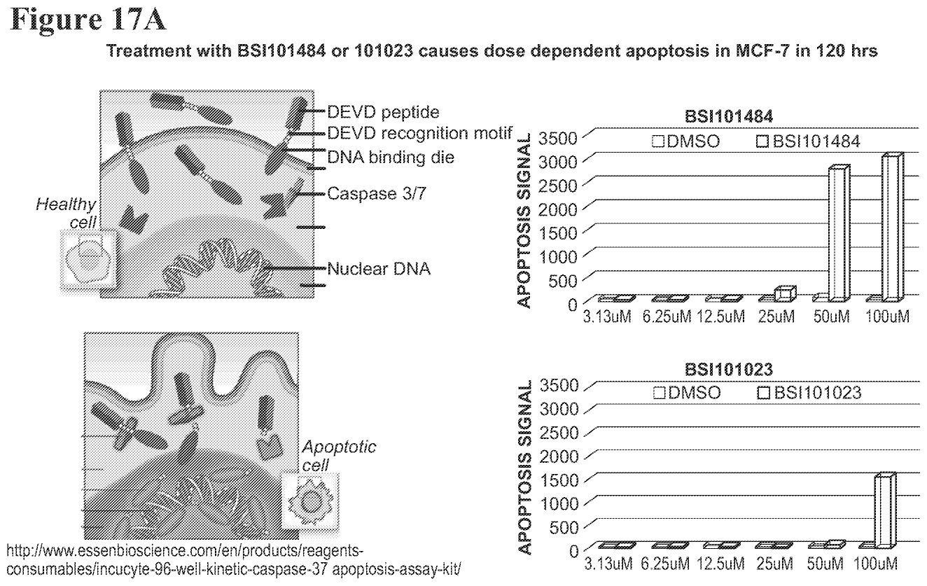

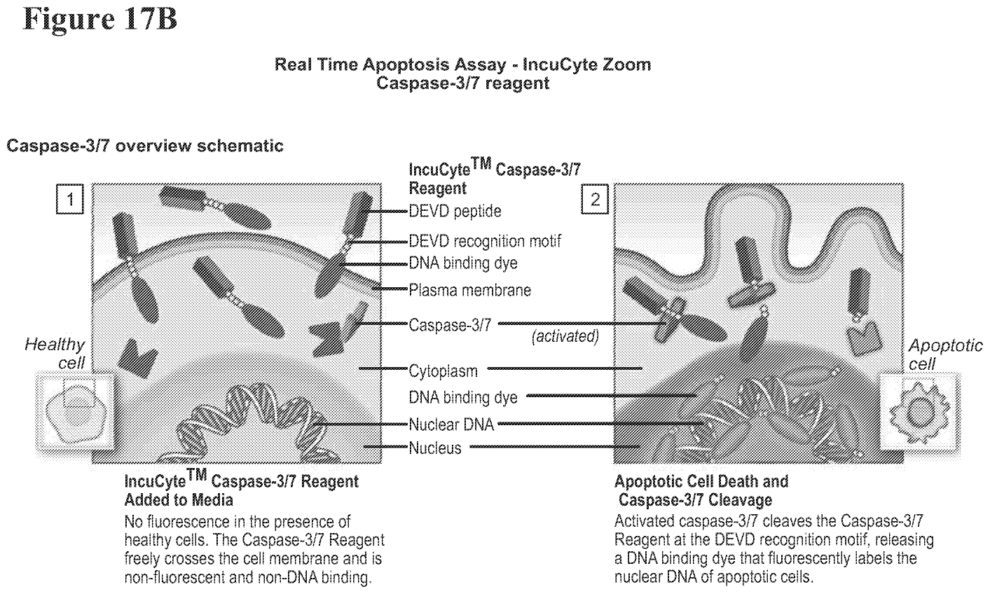

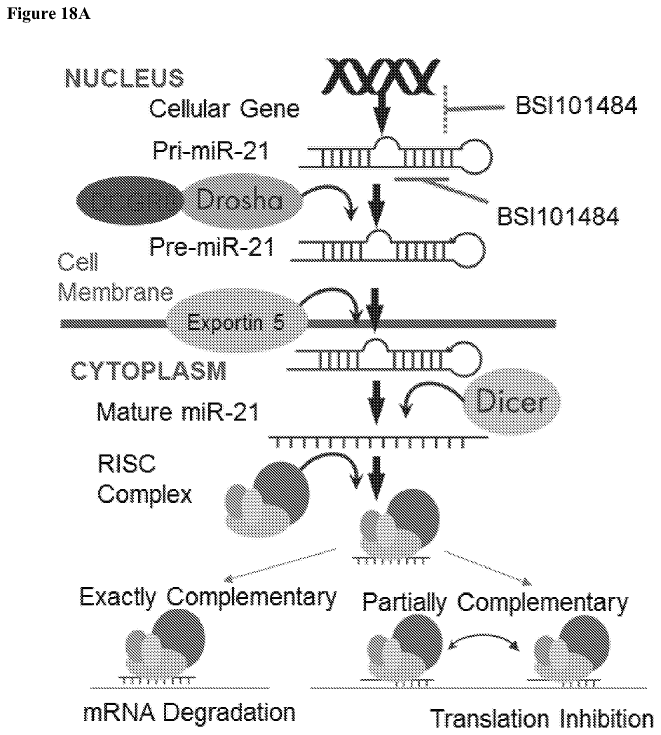

Thus, it was demonstrated that compounds BSI101023 and BSI101484 caused a decline in cell growth in a manner that was dose-dependent as concentrations increased. Furthermore, compounds BSI101023 and BSI101484 increased the expression of Programmed Cell Death 4 (PDCD4), a downstream effector of microRNA-21, as shown in FIGS. 14A, 14B, 16A, 16B, and 37.

### Example 4

### Analysis of the microRNA Pathway

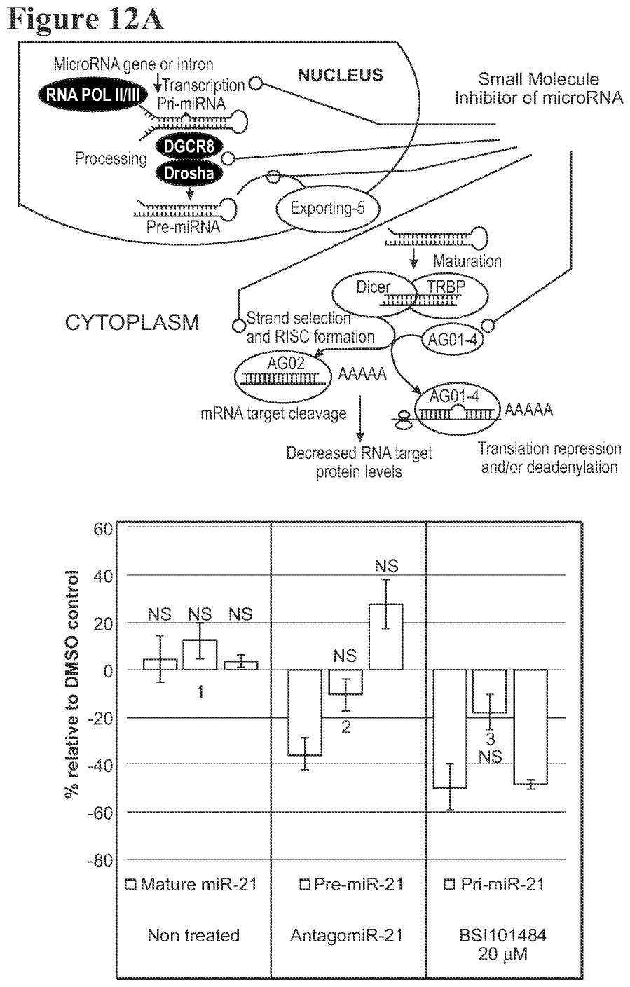

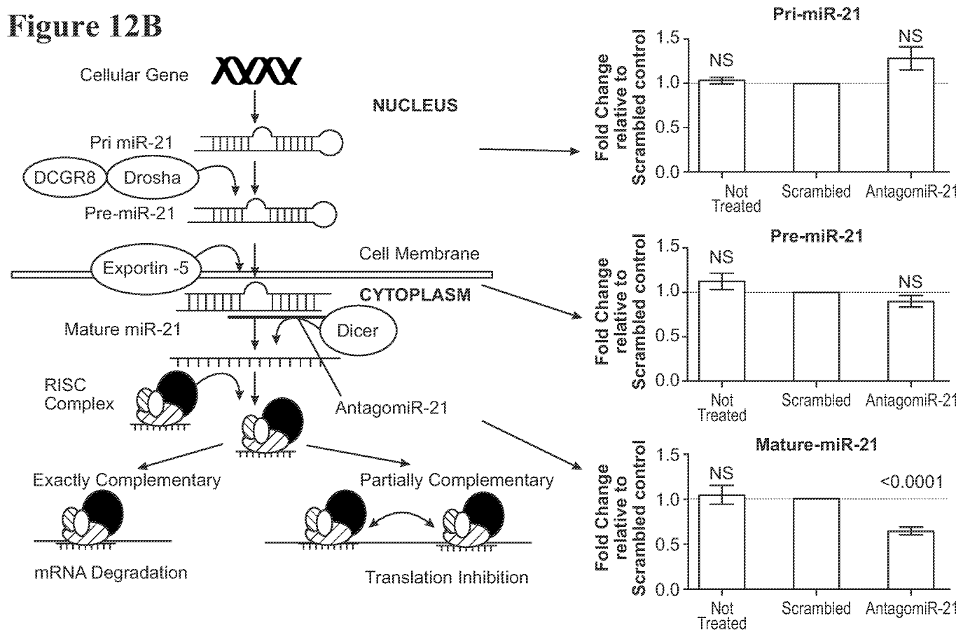

Small molecules can interface with the microRNA maturation pathway at a number of pathway locations. FIG. 12A depicts a schematic of the entry points for small molecules modulating microRNA (e.g., microRNA-21) activity. Compounds can inhibit microRNAs at various stages of the microRNA pathway including: pri-microRNA, pre-microRNA, microRNA duplex, and mature RNA. Inhibition may take place inside the nucleus, in the cytoplasm, or both. FIG. 12B depicts the microRNA pathway and points at which test compounds can act on microRNAs. Histograms depict the results of reporter assays performed upon a test microRNA antagonist (antagomiR, i.e., the antagomiR-21 oligonucleotide agent) known to act upon mature microRNA-21, as compared to untreated and scrambled controls, showing observed levels of pri-microRNA, pre-microRNA, and mature-microRNA (e.g., pri-microRNA-21, pre-microRNA-21, and mature-microRNA-21). Luciferase activity for a random scrambled antagomiR sequence (scrambled) was compared to activity observed for the antagomiR-21 oligonucleotide agent. Such results demonstrated the ability of the antagomiR-21 oligonucleotide agent, as expected, to bind to microRNA-21 and trigger reporter gene expression in the biological functional screening methods described herein. These results also demonstrated that the antagomiR-21 oligonucleotide agent blocked expression of microRNA-21 at the mature-microRNA-21 stage, via triggering of mature-microRNA-21 degradation.

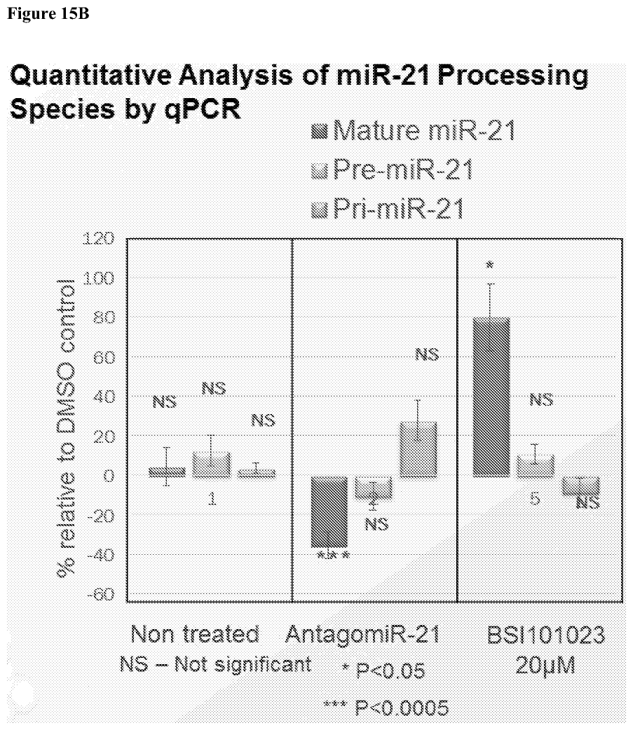

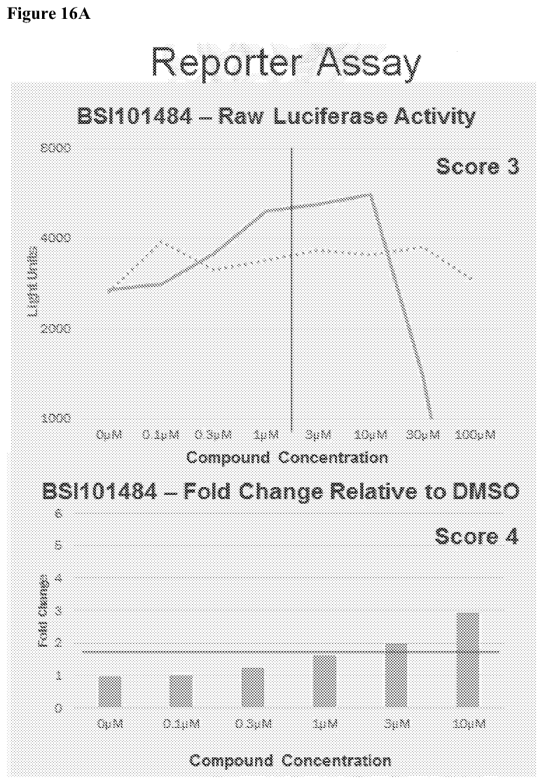

To determine at what stage in the microRNA pathway hit compounds of the above-described assay(s) were acting, quantitative analyses of microRNA processing species was performed upon various hit compounds. Quantitative analysis was performed at IC25 to observe levels of microRNA processing species in the presence versus absence of hit compounds. FIG. 15A depicts a schematic of the microRNA-21 pathway, which shows the point at which the compound BSI101023 appears to act. FIG. 15B depicts the percent expression levels of pri-microRNA-21, pre-microRNA-21, and mature-microRNA-21 observed relative to a DMSO control for respective exposures to no treatment, the antagomiR-21 oligonucleotide agent or to BSI01023 (20 μM). For statistical analyses, * is P<0.05, *** is P<0.0005, and NS is “not significant”. As demonstrated by FIG. 15B, BSI101023 appeared to act at the mature microRNA-21 stage by inhibiting loading of mature microRNA-21 into the RISC complex, thereby creating the significant accumulation of mature microRNA-21 observed for BSI101023 treatment.

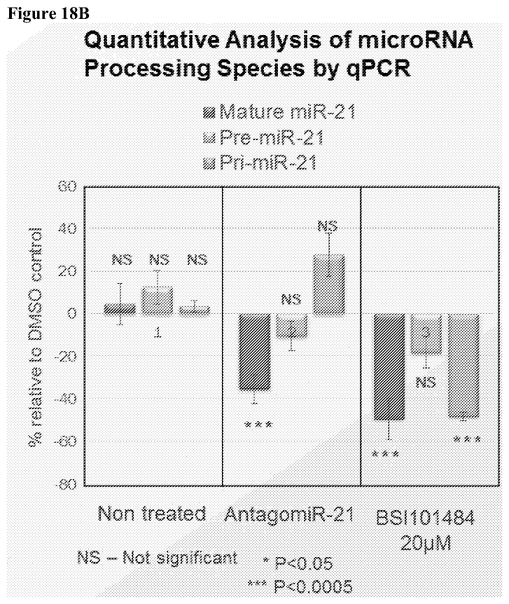

The site of action of BSI101484 was also examined. FIG. 18A depicts a schematic of the microRNA-21 pathway and the point at which the antagomiR-21 compound BSI101484 appears to act. FIG. 18B shows the percent expression levels of pri-microRNA-21, pre-microRNA-21, and mature-microRNA-21 observed relative to a DMSO control for respective exposures to no treatment, the antagomiR-21 oligonucleotide agent or to test compound BSI01484 (20 μM). For statistical analyses, *** is P<0.0005 and NS is “not significant”. As demonstrated in FIG. 18B, BSI101484 appeared to act at the pri-microRNA-21 stage by binding to the loop present in the pri-microRNA-21, to produce an early stage degradation.

Predicted site of microRNA pathway interaction results were obtained for four test compound hits (BSI01023, BSI101484, BSI100945, BSI101534), and quantitative analysis of levels of microRNA processing species were observed at IC25 (data not shown). The percent expression of pri-microRNA-21, pre-microRNA-21, and mature-microRNA-21 relative to DMSO control was assessed following no treatment (negative control) or after treatment with antagomiR-21, BSI01023, BSI101484, BSI100945 or BSI101534, respectively. Compounds BSI101023, BSI101484, BSI101534, and BSI100945 were analyzed at 20 μM, 20 μM, 5 μM, and 20 μM respectively.

### Example 5

### Identification of Candidate Compound Binding Sites Within Targeted microRNAs

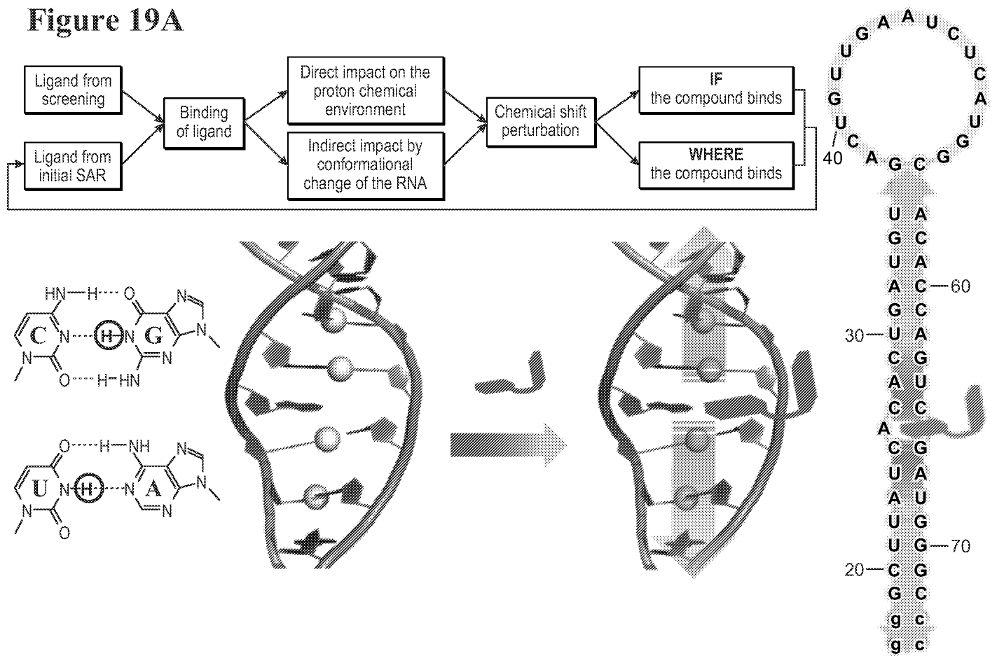

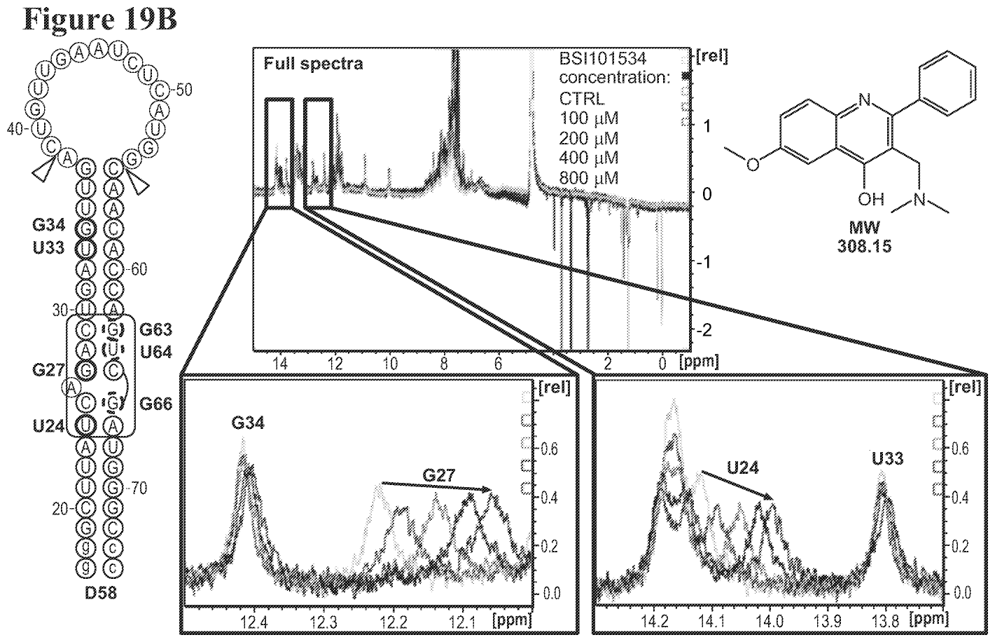

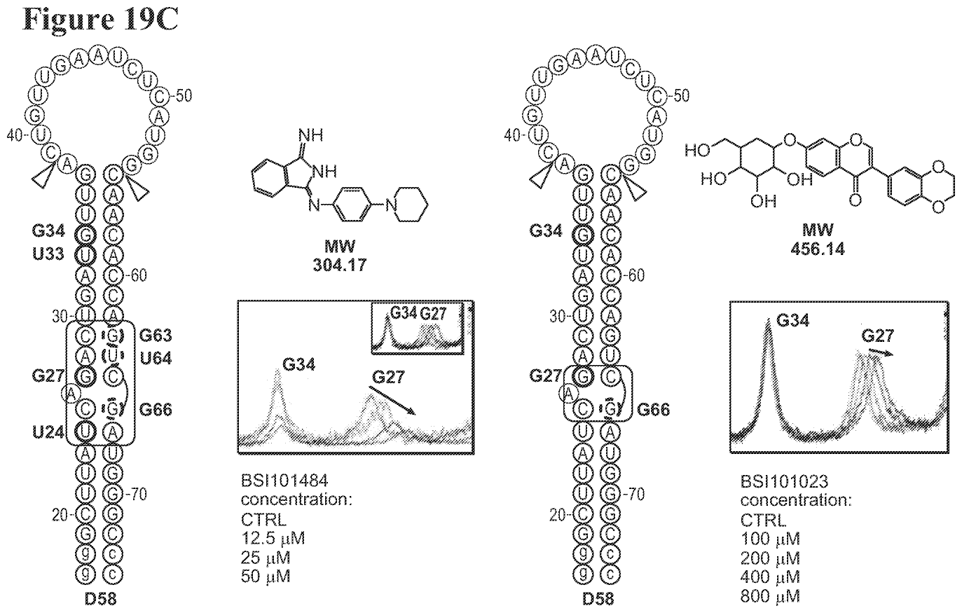

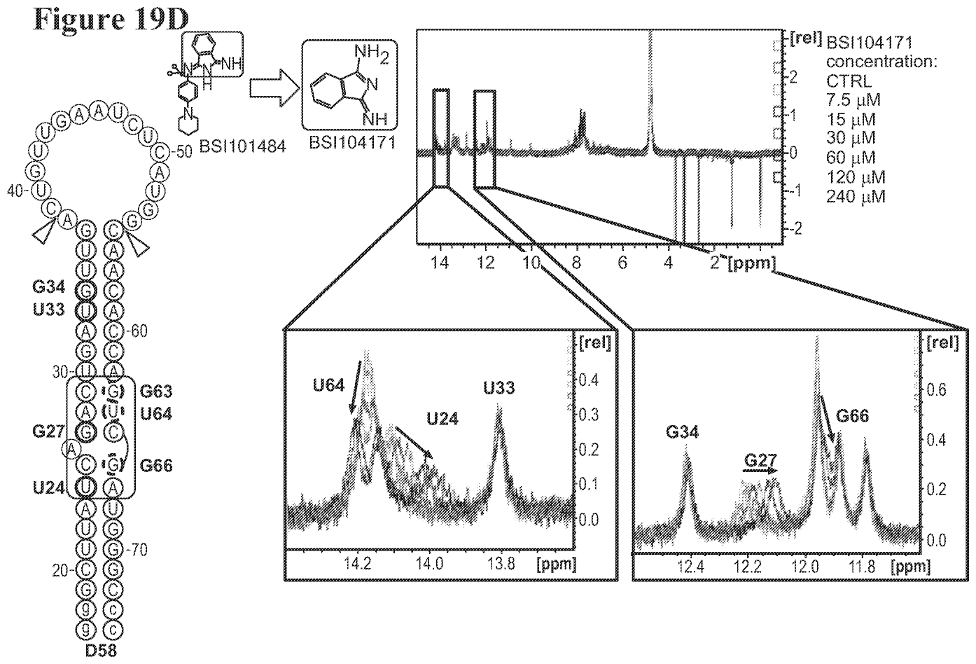

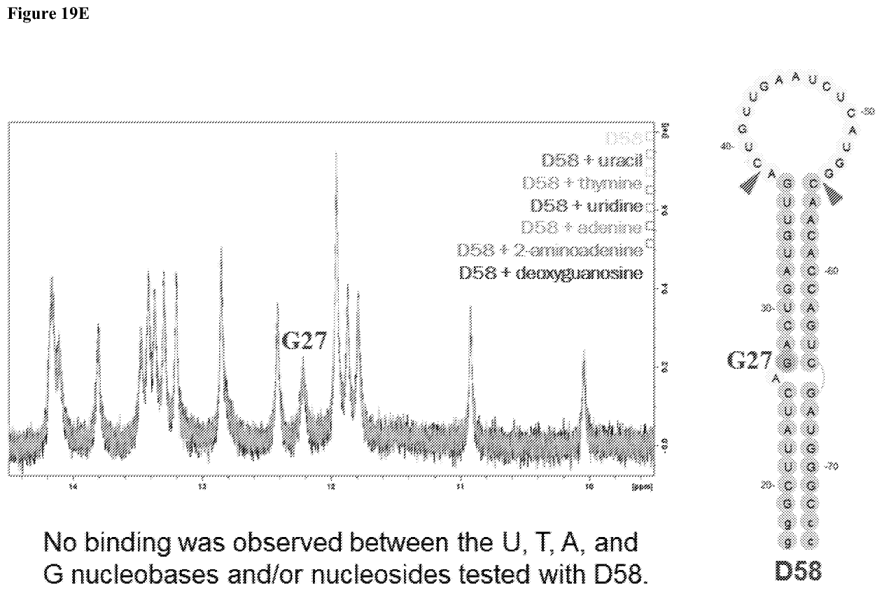

NMR was also used to validate the site of binding of test compounds within targeted microRNA sequences. To prepare RNA for the NMR studies, in vitro transcription was performed and gel purified microRNA was used. The RNA was desalted with a Sep-pak C18 column. After de-salting, the RNA was dried in a genevac evaporator. FIG. 19A in the top panel depicts the scheme employed for performing NMR analysis upon positive screening assay hits (candidate lead compounds). In the bottom panel, the method for mapping the compound binding site is shown. The imino protons (left) are well-resolved in the NMR spectra and assigned to specific bases. Upon binding of a small molecule (center reaction), the local electronic environment is perturbed, both by the presence of the compound and possible structural rearrangements. This causes the local chemical shift environment to change (red in hairpin on the right), causing peaks shifts in the NMR spectrum of the RNA. FIG. 19B depicts a microRNA-21 stem loop structure and associated NMR spectra obtained when candidate lead compound BSI101534 was administered and assayed. BSI101534 was tested for microRNA binding at concentrations of 100 μM, 200 μM, 400 μM, and 800 μM. As demonstrated in the stem loop diagram and the NMR spectra obtained, there was a shift of NMR spectra, consistent with BSI101534 binding microRNA-21. Furthermore, dose-responsiveness was observed, as shown. These results demonstrated that BSI101534 bound only to the Dicer-containing construct, at the A26 bulge region. FIG. 19C depicts the microRNA stem loop structure and NMR spectra obtained for test compounds BSI101484 and BSI101023. BSI101023 was tested at concentrations of 100 μM, 200 μM, 400 μM, and 800 μM. BSI101484 was tested at concentrations of 12.5 μM, 25 μM, and 50 μM. As depicted in FIG. 19C, compounds BSI101484 and BSI101023 also bound in the A26 bulge region. A fragment of candidate lead compound BSI101484, called BSI104171, was also generated and tested for microRNA binding. BSI104171 was analyzed at concentrations of 7.5μM, 15 μM, 30 μM, 60 μM, 120 μM, and 240 μM. FIG. 19E shows a stem loop structure and associated NMR spectra obtained when free, naturally occurring nucleobases were administered to the D58 construct as a control. No binding was observed between the U, T, A, and G nucleobases and/or nucleosides tested in the presence of the D58 structure. Thus, candidate lead compounds BSI10148, BSI104171, BSI101023, etc. were not simply acting as free nucleobases in their respective interactions with the D58 structure.

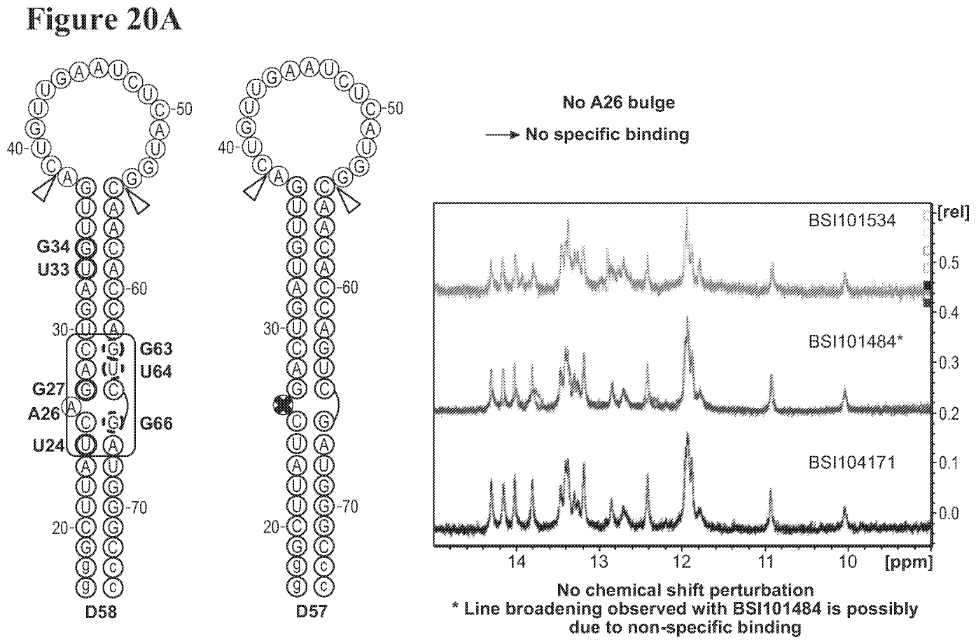

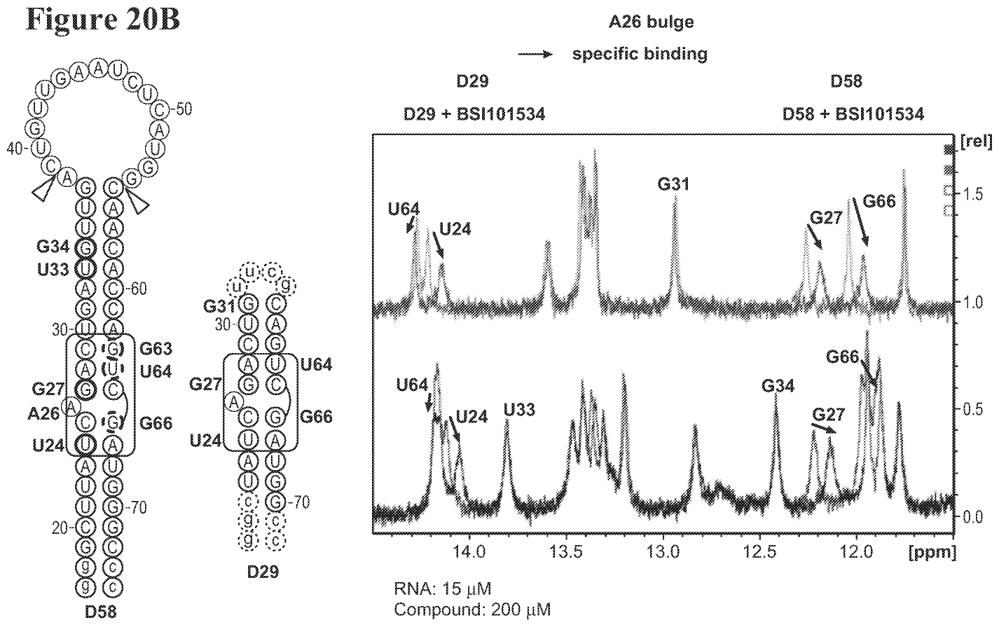

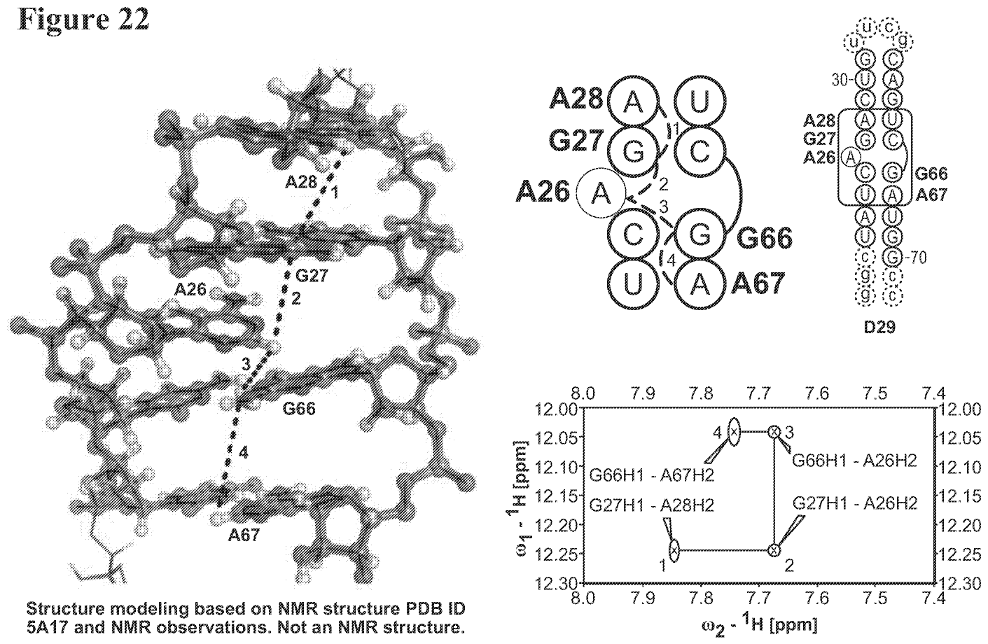

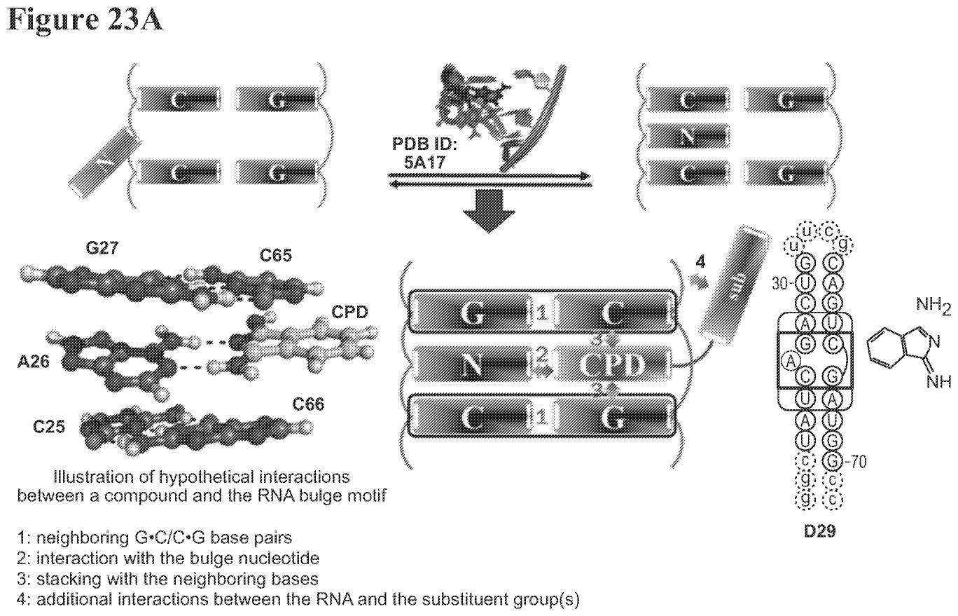

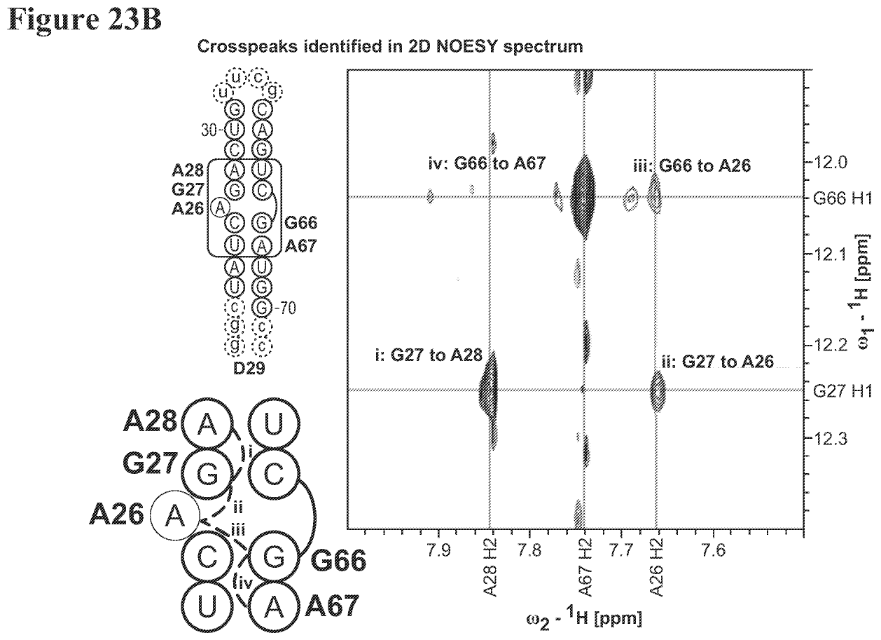

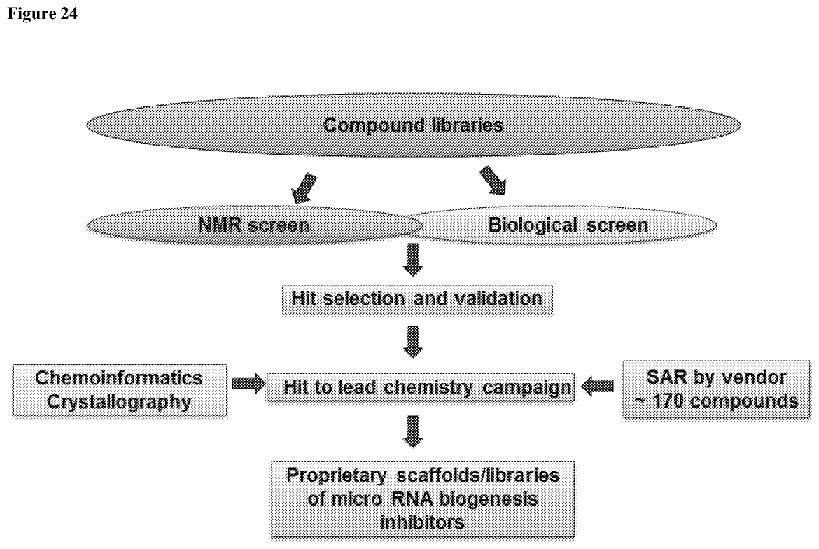

Because the candidate lead compounds assayed by NMR were identified to bind microRNAs in the bulge region, NMR was used to further examine the role of the A26 bulge region in microRNA stability, thereby further refining understanding of the precise site and mechanism of interaction of such candidate lead compounds with the bulge region sequence. Structural features of the bulge region sequence are depicted in FIG. 22. FIG. 20A depicts the effect of removing the A26 bulge from a stem loop structure that was exposed to test compounds BSI101534, BSI101484, and BSI104171. “D57”-associated NMR spectra, which showed no chemical shift perturbation in the NMR spectra and therefore no binding, demonstrated that no specific binding of such test compounds occurred when the A26 bulge region was altered to remove the bulge nucleotide. Criticality of the bulge nucleotide to BSI101534, BSI101484, and BSI 104171 binding was therefore confirmed. Meanwhile, FIG. 20B depicts NMR spectral shifts observed upon administration of candidate lead compound BSI101534 to indicated “D58” and “D29” structures, where the “D29” structure eliminates the loop region of microRNA-21, replacing it with only a tetraloop, yet binding of BSI101534 to the bulge region sequence of this “D29” structure was unaltered. BSI101534 at 200 μM was reacted with RNA at 15 μM. Thus, changing the length of the microRNA yet retaining the A26 bulge region demonstrated that A26 bulge sequence was sufficient for BSI101534 (and likely other candidate lead compounds) to bind microRNA (here, microRNA-21).

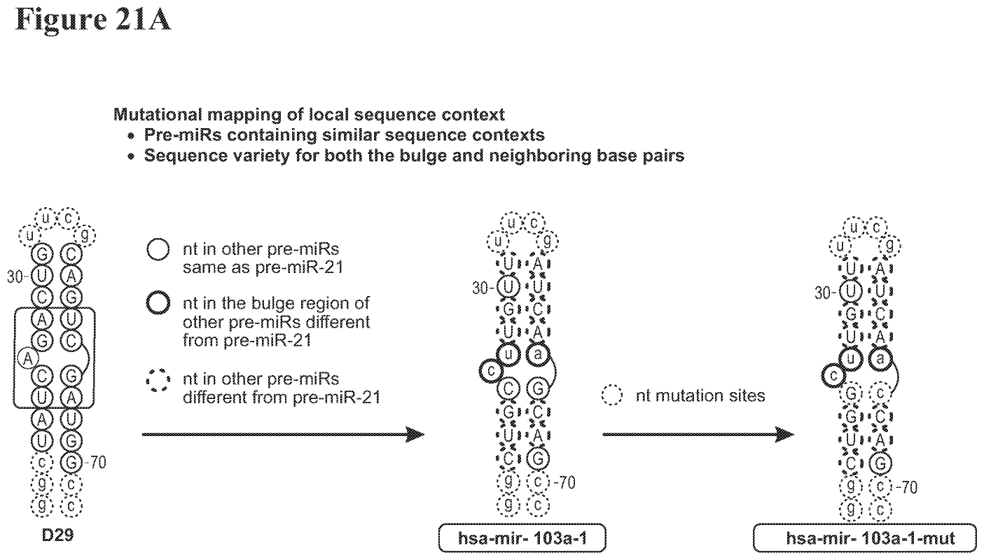

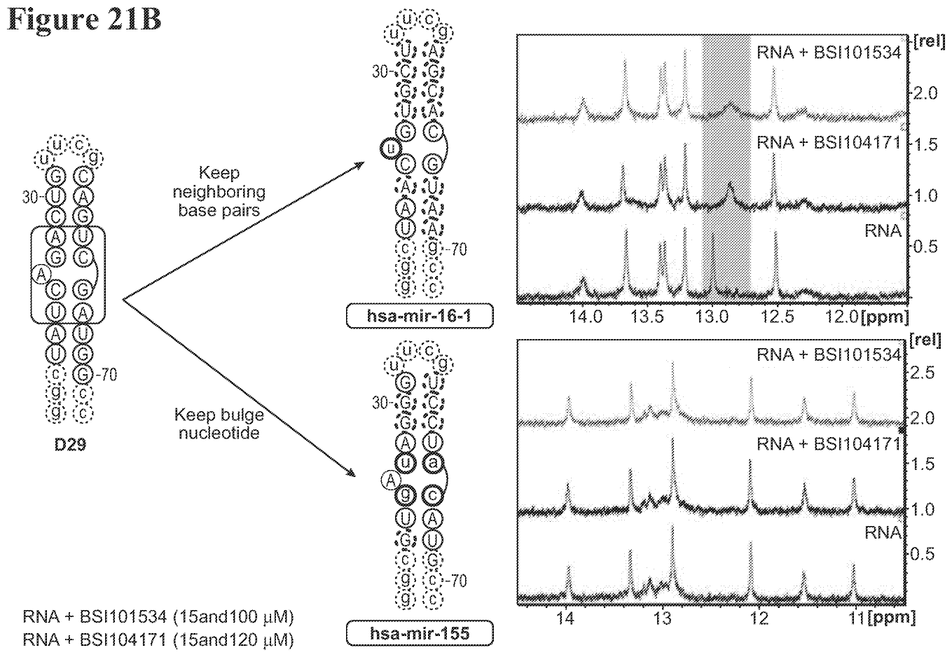

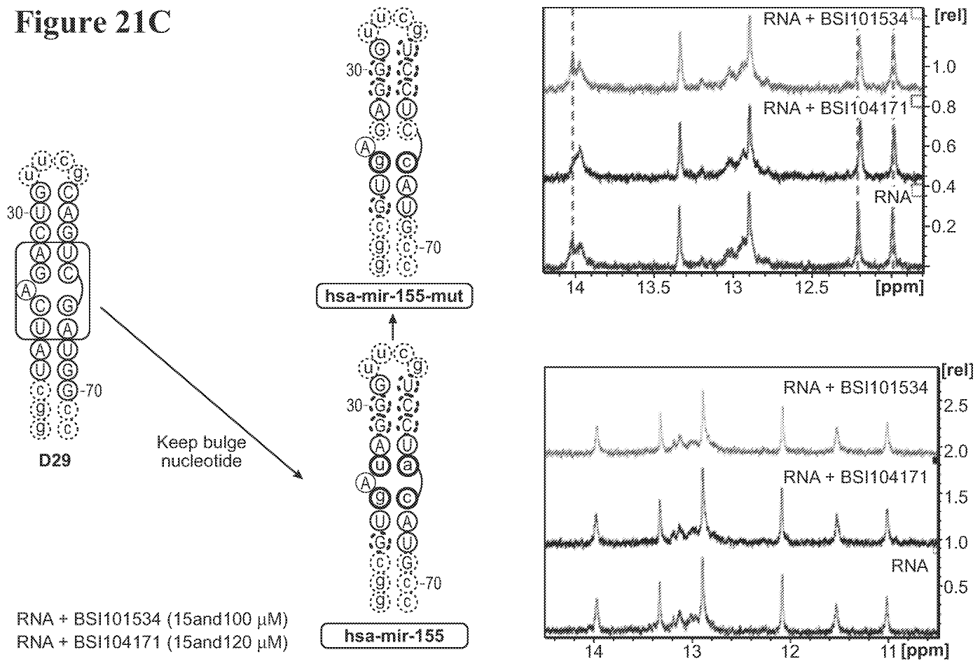

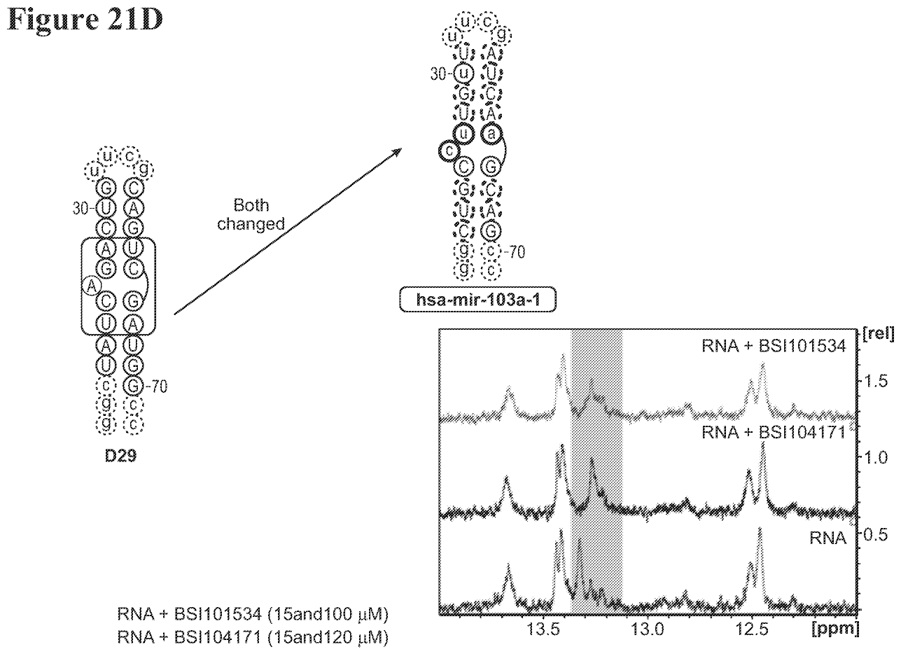

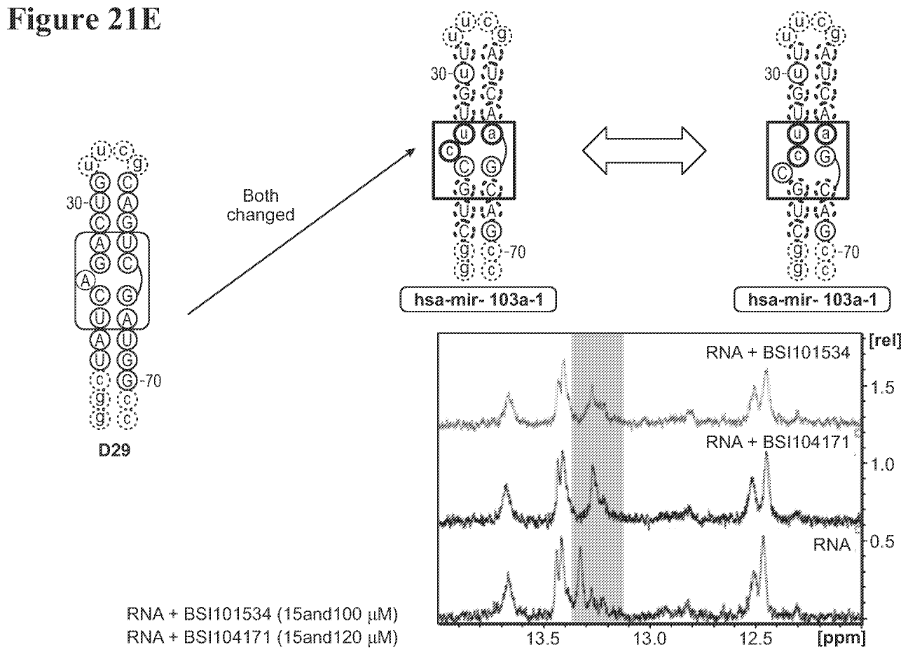

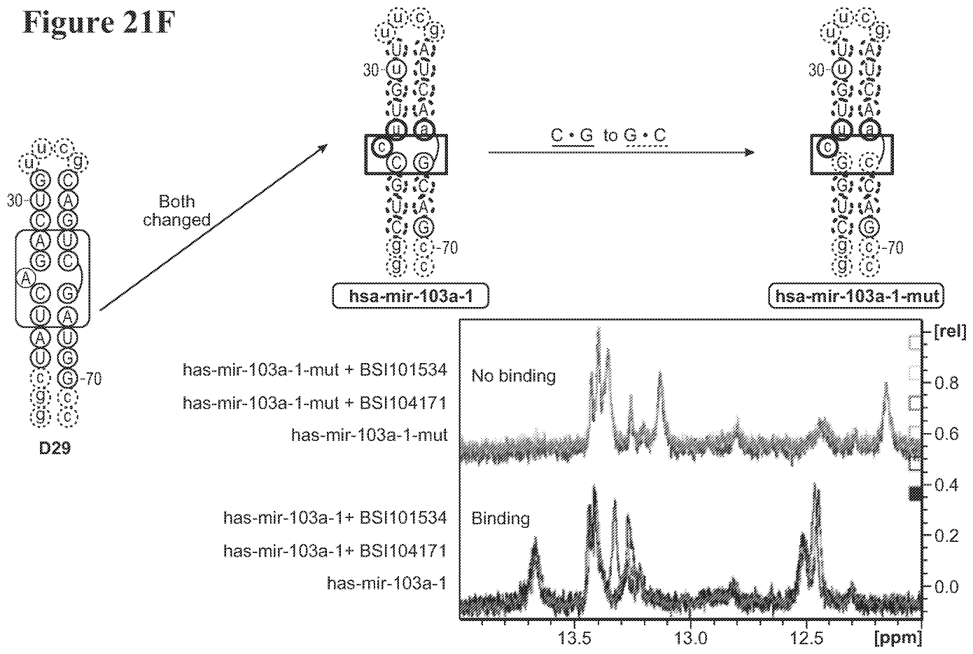

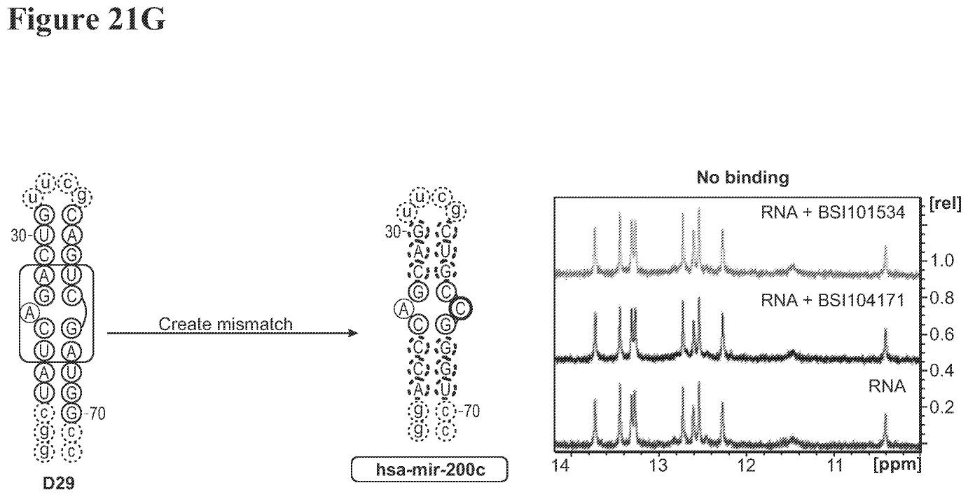

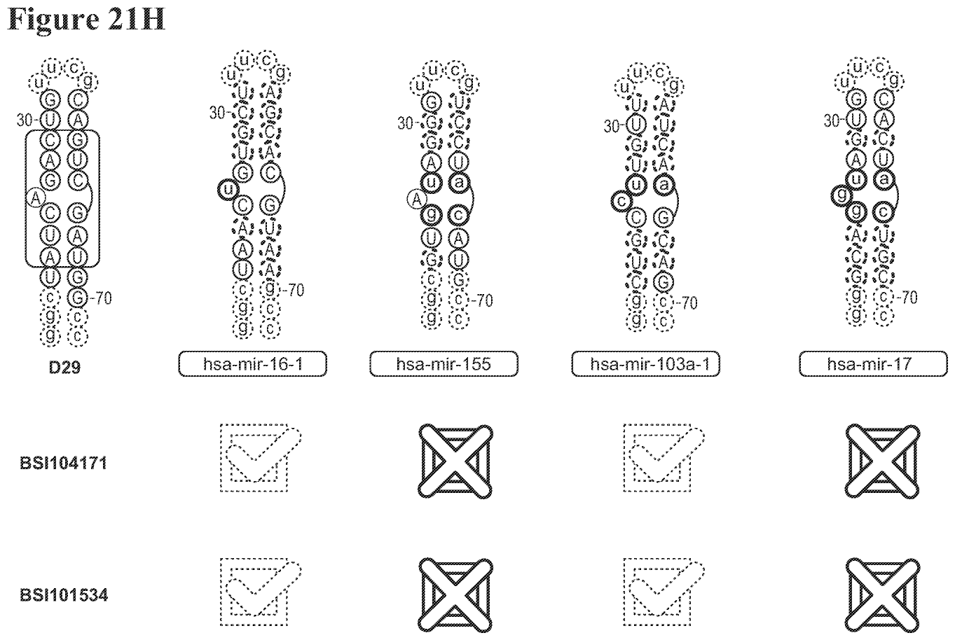

NMR titration was also used to examine whether the binding of candidate lead compounds to Dicer cleavage site-containing microRNAs was sequence-context dependent. FIG. 21A depicts the scheme employed for mutational mapping of the candidate lead compound binding site(s). Alteration of the bulge sequence region of microRNA-21 was performed via introduction of corresponding sequence regions derived from other microRNAs, as well as introduction of point mutations into such constructs. In altered microRNA-21-derived constructs, sequences in the bulge region were substituted to mimic bulge regions from non-microRNA-21s (in FIG. 21A, hsa-mir-103a-1 and a mutant form thereof, as shown). Additionally, sequences outside the bulge region were altered to mimic the non-bulge regions of other microRNAs (microRNAs other than microRNA-21). FIG. 21B shows alternate stem loop schemes and NMR spectra for microRNA-21-derived constructs where 1) the neighboring base pairs remain but the bulge is altered (“hsa-mir-16-1”) and 2) neighboring base pairs are altered while the bulge region is altered, yet the bulge nucleotide is retained (“hsa-mir-155”). NMR spectra for the different altered microRNA-21-derived constructs (15 μM) mixed with BSI101534 (100 μM) and BSI104171 (120 μM) are depicted—peaks that shift for hsa-mir-16-1 (retaining neighboring base pairs) upon addition of BSI101534 and BSI104171 but not in controls is shaded. FIG. 21C depicts alternate stem loop schemes and NMR spectra for microRNA-21-derived constructs where the construct from FIG. 21B possessing hsa-mir-155 bulge region with un-altered bulge nucleotide was compared to the same construct possessing an altered bulge region sequence (“hsa-mir-155-mut”). NMR spectra for the altered construct (15 μM) and its variant with BSI101534 (100 μM) and BSI104171 (120 μM) are depicted. FIG. 21D depicts stem loop structures and NMR spectra for microRNA-21-derived constructs where both the bulge region and sequences outside the bulge region are altered to those of hsa-mir-103a-1. NMR spectra for the altered construct (15 μM) with BSI101534 (100 μM) and BSI104171 (120 μM) are depicted. FIG. 21E depicts stem loop structures and NMR spectra for the microRNA-21-derived hsa-mir-103a-1 construct of FIG. 21A, where both the bulge region and sequences outside the bulge region are altered with nucleotides of hsa-mir-103a-1. NMR spectra for the hsa-mir-103a-1 construct (15 μM) with BSI101534 (100 μM) and BSI104171 (120 μM) revealed wobble at the bulge residue of this construct, which effectively shifted the location of the bulge within this construct between two states as shown. FIG. 21F depicts confirmation of the effect of the wobble observed in stem loop structures of FIG. 21E, where blocking of such wobble via mutation (in the “hsa-mir-103a-1-mut” construct) was revealed to block binding of microRNA-binding test compounds. The bottom NMR traces of FIG. 21F show constructs of hsa-mir-103a-1 (15 μM) that allowed both wobble and binding of BSI101534 (100 μM) and BSI104171 (120 μM), whereas the top NMR traces of FIG. 21F show that BSI101534 and BSI104171 no longer bound the “hsa-mir-103a-1-mut” construct (altered such that relative position of a C-G base pair within the duplex was swapped across the duplex). FIG. 21G depicts stem loop structures and NMR spectra for a microRNA-21-derived structure having the hsa-mir-200c bulge region sequence, which possesses a mismatch. NMR spectra for the altered microRNA-21 construct (15 μM) with BSI101534 (100 μM) and BSI104171 (120 μM) are depicted. No binding was witnessed for either compound. These results demonstrated that the candidate lead compounds identified in the parallel screen described above all bound microRNA-21 in a sequence-dependent manner, and specifically appeared to do so by stabilizing a specific wobble position within the microRNA-21 bulge region sequence (effectively intercalating into the dsRNA structure and interacting with bulge nucleotides in a structurally stabilizing manner).

Illustrations of predicted interactions between a compound and the RNA bulge motif are depicted in FIG. 27. Such illustrations demonstrate that screened compounds likely interacted with the RNA bulge motif through mechanisms including: association with neighboring GC/CG base pairs; interactions with the bulge nucleotide; stacking with neighboring bases; and additional interactions between the RNA and the substituent group.

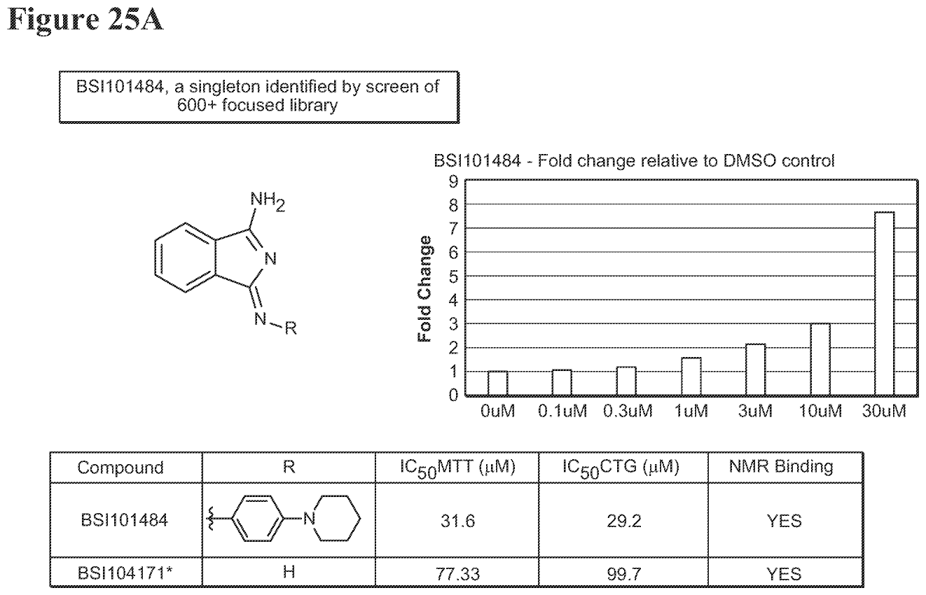

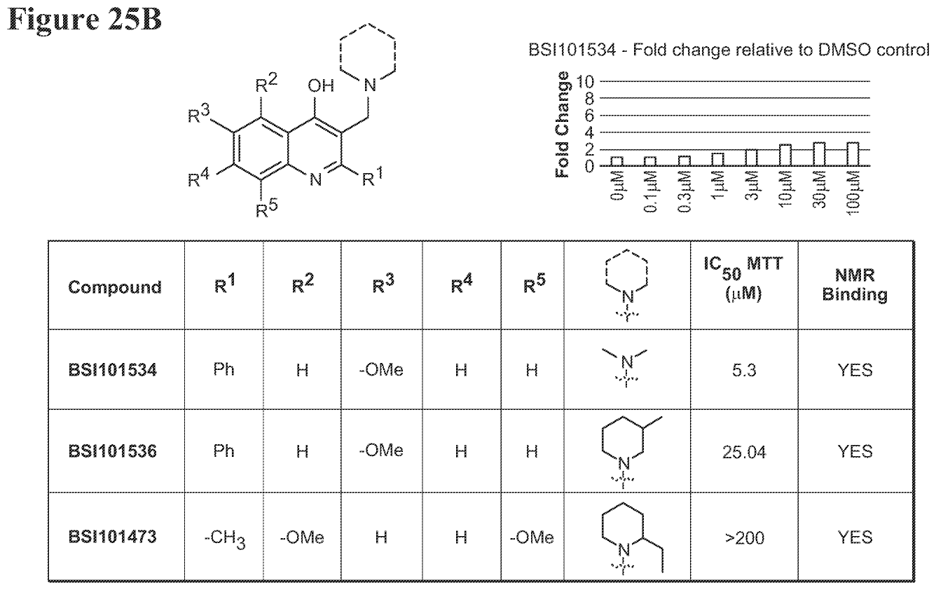

Screening results for BSI101484 and related compound BSI104171 were also compiled and compared, as depicted in FIG. 25A. FIG. 25B depicts the structure and fold changes observed relative to DMSO for compound BSI101534, as well as for other related compounds. Screening assay results for BSI101534, BSI101536, and BSI101473 are also presented in FIG. 25B. FIGS. 25A and 25B therefore demonstrate that BSI101484, BSI104171, BSI101534, BSI101536, and BSI101473 compounds all exhibited microRNA binding in the NMR titration studies, as well as biological activities associated with such binding. Notably, BSI101484 exhibited a higher fold change relative to DMSO at a lower concentration, as compared to BSI101534. Such results demonstrate that additional optimization of binding and/or efficacies can be performed upon candidate lead compounds initially identified via the above-described screening approaches.

### Example 6

### SAR Expansion and Validation

Structure activity relationships (SARs) between identified positive hits (candidate lead compounds) and microRNA molecules can also be further analyzed and validated. In the present Example, test compounds BSI101484 and BSI101534 are evaluated to determine the contribution of each substituent to the interaction of these compounds with microRNA molecules. Structural features of the test compound are altered and the effect exerted upon test compound binding of microRNAs is examined in preliminary in vitro and toxicity assays.

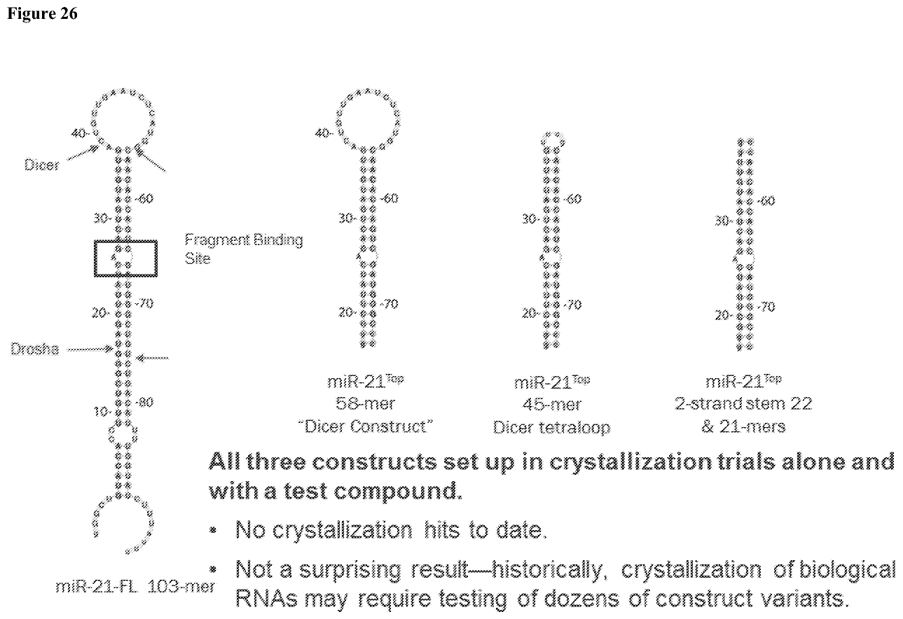

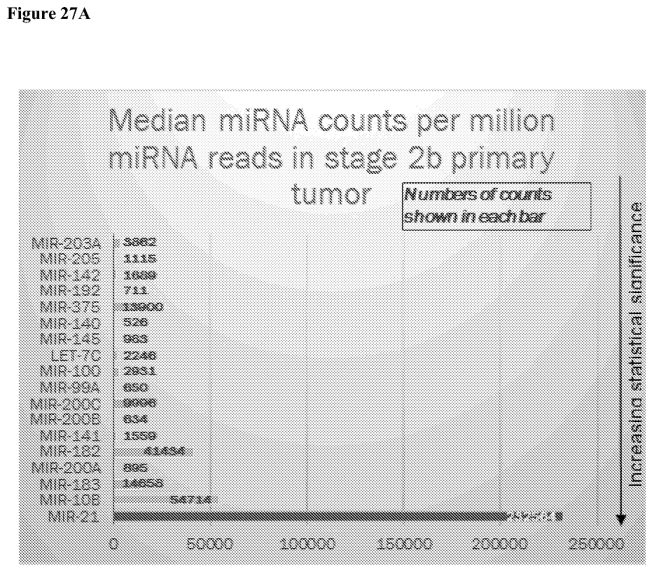

Crystallographic analysis of lead compound binding to microRNAs are employed to further complement the chemical and biological screening methods described above. Crystallography sheds light upon the relevant specific atoms responsible for the binding interaction between microRNAs and candidate lead compounds identified in the screens described herein. FIG. 26 depicts stem loop structures of microRNA-21 constructs that have been prepared for crystallization assays. Such structures are ultimately derived from the indicated 103-mer microRNA-21 full length construct, and the three microRNA-21-derived constructs shown were set up for crystallization. These included a microRNA-21 58-mer Dicer construct, a microRNA-21 45-mer Dicer tetraloop, and a microRNA-21 two strand stem of 21 and 22-mers. Crystals, once formed, are assessed using crystallography approaches known in the art, thereby further refining identification of the specific site(s) of interaction of the candidate lead compounds with targeted RNA sequences.

### Equivalents

Those skilled in the art will recognize, or be able to ascertain using no more than routine experimentation, many equivalents to the specific embodiments of the disclosure described herein. Such equivalents are intended to be encompassed by the following claims.

1. A method for identifying a candidate lead compound comprising:
(a) contacting an oligonucleotide with a test compound in a biological functional assay, wherein oligonucleotide-test compound binding results in a biological result not observed in the biological functional assay without the test compound; and
(b) contacting the oligonucleotide with the test compound, thereby forming an oligonucleotide-test compound solution; performing NMR upon the oligonucleotide-test compound solution, wherein oligonucleotide-test compound binding produces an NMR result not observed in a solution lacking said test compound; and detecting said NMR result in the oligonucleotide-test compound solution, wherein detecting selective biological function in the presence of the test compound in (a) and detecting said NMR result in (b) identifies the test compound as a candidate lead compound.

2. The method of claim 1, wherein the biological functional assay is selected from the group consisting of a cell-based reporter system, a proliferation assay, an immunoassay or a polymerase chain reaction-based assay.

3. The method of claim 1, wherein the oligonucleotide is selected from the group consisting of coding and non-coding RNAs, optionally wherein the oligonucleotide is selected from the group consisting of a microRNA, a tRNA, a rRNA, a tiRNA, a lincRNA, a NAT, a lncRNA, a, eRNA, a T-UCR, a circRNA, a piRNA, an esiRNA, an siRNA, an antisense oligonucleotide, a tasiRNA, a snoRNA, a scaRNA and a snRNA.

4. The method of claim 3, further comprising assessing the site of microRNA processing pathway activity of the candidate lead compound by measuring levels of pri-microRNA, pre-microRNA and mature microRNA, as compared to an appropriate control.

5. The method of claim 1, wherein the oligonucleotide is a microRNA-21 transcript or a fragment thereof comprising at least 15 consecutive nucleotides of microRNA-21 and optionally comprises the Drosha and/or Dicer cleavage site.

6. The method of claim 1, wherein detecting the biological result of the biological functional assay in the presence of the test compound in step (a) comprises identifying at least a 1.5-fold elevation of the level of a signal in the presence of the test compound, relative to the level of the signal in the absence of the test compound, optionally wherein detecting the biological result of the biological functional assay in the presence of the test compound in step (a) comprises identifying at least a 1.75-fold elevation of the level of a signal in the presence of the test compound, relative to the level of the signal in the absence of the test compound, optionally wherein detecting the biological result of the biological functional assay in the presence of the test compound in step (a) comprises identifying at least a two-fold elevation of the level of a signal in the presence of the test compound, relative to the level of the signal in the absence of the test compound.

7. The method of claim 1, wherein the test compound is assigned (i) an integer score based upon the shape of a dose-response curve for the biological functional assay in the presence of the test compound and (ii) an integer score based upon the dose-responsiveness of the biological functional assay to the test compound.

8. The method of claim 7, wherein the integer score of (i) is between 0 and 3, wherein 0 is given to test compounds that show no signal; 1 indicates a signal only at higher concentrations; 2 indicates a signal proportional to its concentration; and 3 indicates a signal at low concentrations; and the integer score of (ii) is between 0 and 7, with the higher value assigned when a signal is shown at lower concentrations.

9. The method of claim 1, wherein detecting said NMR result in the oligonucleotide-test compound solution in (b) comprises assigning a numeric score to the oligonucleotide-test compound NMR results.

10. The method of claim 9, wherein a score of 0, 1, 2, 3 or 4 is assigned to the oligonucleotide-test compound NMR results, wherein a score of 4 indicates high-quality NMR-detected binding, optionally wherein scoring is assigned as follows:
+1 for signal demonstrating binding;
+1 for signal-to-noise ratio >3;
+1 for sharp ligand peaks in the mixture;
+1 for pattern of ligand peaks consistent with the pattern expected from the chemical structure.

11. The method of claim 8, wherein the combined scores of the biological functional assay and the NMR assay of 10 or greater identifies the test compound as a compound that binds the oligonucleotide and/or is bioactive.

12. The method of claim 1 comprising:
(a) contacting an oligonucleotide with a test compound in the presence of a cell-based reporter system; and detecting selective activation of the cell-based reporter system in the presence of the test compound; and
(b) contacting the oligonucleotide with the test compound, thereby forming an oligonucleotide-test compound solution; performing NMR upon the oligonucleotide-test compound solution, wherein oligonucleotide-test compound binding produces an NMR result not observed in a solution lacking said test compound; and detecting said NMR result in the oligonucleotide-test compound solution,
wherein detecting selective activation of the cell-based reporter system in the presence of the test compound in (a) and detecting said NMR result in (b) identifies the test compound as a candidate lead compound.

13. A method for validating a candidate lead compound and identifying the site of candidate lead compound-microRNA interaction comprising:
identifying binding of a candidate lead compound to a microRNA or a microRNA fragment;
altering the sequence of the microRNA or microRNA fragment via introduction of one or more point mutations, thereby generating a mutated microRNA or a mutated microRNA fragment; and
identifying absence of binding of the candidate lead compound to the mutated microRNA or mutated microRNA fragment according to the method of any of the preceding claims,
thereby validating the candidate lead compound and identifying the site of candidate lead compound-microRNA interaction.

14. A method for validating a candidate lead compound and identifying the site of activity of the candidate lead compound within the microRNA pathway comprising:
identifying binding of a candidate lead compound to a microRNA or a microRNA fragment according to any of the methods of the preceding claims;
assaying levels of pri-microRNA, pre-microRNA and mature microRNA in the presence of the candidate lead compound, as compared to in the absence of the candidate lead compound; and
identifying the site of activity of the candidate lead compound within the microRNA pathway based upon the relative levels of pri-microRNA, pre-microRNA and mature microRNA assayed in the presence of the candidate lead compound, as compared to in the absence of the candidate lead compound,
thereby validating the candidate lead compound and identifying the site of activity of the candidate lead compound within the microRNA pathway.

15. The method of claim 1, wherein the test compound is a small molecule.
